(19) United States
(12) Patent Application Publication
(10) Pub. No.: US 2005/0282734 A1
(43) Pub. Date: Dec. 22, 2005

(54) Pharmaceutically acceptable composition comprising an aqueous solution of paclitaxel and albumin

(76) Inventors: Tenshuk A. Kadima, Winnipeg (CA); Howard A. Kaplan, Winnipeg (CA); Robert C. Tuttle, Winnipeg (CA); Lajos Hegedus, Budapest (HU); Krisztina Krempels, Budapest (HU); Krisztina Paal, Budapest (HU); Gabor Petho, Budapest (HU)

Correspondence Address: KENYON & KENYON, ONE BROADWAY, NEW YORK, NY, 10004, US

(21) Appl. No.: 11/088,280

(22) Filed: Mar. 23, 2005

Related U.S. Application Data
(63) Continuation of application No. 10/881,824, filed on Jun. 28, 2004, now abandoned.
Application No. 10/881,824 is a continuation of application No. 10/657,737, filed on Sep. 3, 2003.
Application No. 10/657,737 is a continuation of application No. 10/293,426, filed on Nov. 12, 2002, now abandoned.
Application No. 10/293,426 is a continuation of application No. 09/947,420, filed on Sep. 5, 2001, now abandoned.
Application No. 09/947,420 is a continuation of application No. 09/744,827, now abandoned.
(60) Provisional application No. 60/094,687, filed on Jul. 30, 1998.

(51) Int. Cl. A61K 38/38; A61K 31/337

(52) U.S. Cl. 514/2; 514/449

(57) ABSTRACT

An optically clear, pharmaceutically acceptable aqueous composition comprising paclitaxel or a derivative thereof, serum albumin and a pharmaceutically acceptable vehicle, wherein the composition comprises no more than 10% organic solvent and has a pH of about 3.0 to about 4.8, is described. The serum albumin can be fatted or defatted, and the composition can optionally be lyophilized or optionally lyophilized and reconstituted. At least 70% of the paclitaxel is bound to serum albumin, the ratio of paclitaxel to albumin is at least about 1:5, and the concentration of paclitaxel is at least about 25 μg/ml. Methods of making and using this composition an also provided.

### CROSS-REFERENCE TO RELATED APPLICATIONS

Not Applicable

### STATEMENT OF RIGHTS TO INVENTIONS MADE UNDER FEDERALLY-SPONSORED RESEARCH

Not Applicable

### TECHNICAL FIELD

The present invention relates generally to aqueous formulations of paclitaxel and methods of use thereof. More specifically, it pertains to pharmaceutical compositions comprising paclitaxel (Ptx) or a derivative thereof and serum albumin or a fragment thereof, particularly human serum albumin, and more particularly recombinant human serum albumin, and a physiologically acceptable vehicle; methods of preparation of such pharmaceutical compositions; and methods of use thereof. The vehicle can comprise an organic solvent, and the composition lacks a toxic emulsifier such as Cremophor EL® (polyoxyethylated castor oil).

### BACKGROUND OF THE INVENTION

Paclitaxel, a structurally complex natural plant product, has demonstrated efficacy in the treatment of a wide variety of human malignancies. This drug shows strong cytotoxicity in KB cell structures and in several of the National Cancer Institute's in vivo screens, including the P-388, L-1210, and P-1534 mouse leukemias, the B-16 melanocarcinoma, the CX-1 colon xenograft, the LX-1 lung xenograft, and the MX-1 breast xenograft. Further, studies by McGuire et al. [(1989) Ann. Int. Med. 111:273-279] found paclitaxel to be active against drug-refractory ovarian cancer. Positive results were also seen with paclitaxel treatment of patients with other cancers, including melanoma Einsig et al. (1988) Proc. Am. Soc. Clin. Oncol. 7:249; Holmes (1991) J. Natl. Cancer Inst. 83:1797-1805; and Kohn et al. (1994) J. Natl. Cancer inst. 86:18-24.

In addition to various cancers, paclitaxel has been used in treating several other diseases, including malaria and babesiosis. U.S. Pat. Nos. 5,356,927 and 5,631,278. Paclitaxel can be used to treat indications characterized by chronic inflammation such as rheumatoid arthritis and auto-immune disease. U.S. Pat. No. 5,583,153; and Song et al. (1996) Arthritis Rheum. 39:S 178. Paclitaxel can impair chronic inflammation by inhibiting the activity of white blood cells involved in the inflammatory response; reducing the production of matrix metalloproteinases that permanently damage tissues; blocking the cancer-like growth of previously normal cells which respond to chronic inflammation by proliferating; and inhibiting the growth of blood vessels which lead to the formation of scar tissue. Paclitaxel is also a potent inhibitor of angiogenesis and other processes involved in the development of chronic inflammation. This activity is due, in part, to paclitaxel's ability to inhibit the transcription factor AP-1. AP-1 is a key regulator of genes involved in the production of (i) matrix metalloproteinases, (ii) cytokines associated with chronic inflammation, and (iii) proteins necessary for cell proliferation. Therefore, paclitaxel inhibits a regulator which plays an important role in chronic inflammation and conditions that are dependent on angiogenesis (new blood vessel formation), including tumor growth. Paclitaxel has shown strong anti-angiogenic activity when tested in the chorioallantoic membrane of the developing chick embryo. The drug is a more potent angiogenesis inhibitor than approved anti-arthritic agents such as methotrexate, penicillamine, and steroids.

Atherosclerosis and restenosis have also been treated with low paclitaxel dosages. U.S. Pat. No. 5,616,608. Paclitaxel can alter several aspects of the process leading to restenosis, including inhibition of vascular smooth muscle cell (“VSMC”) migration, inhibition of VSMC proliferation, and inhibition of the effects of certain growth factors on these cells. Paclitaxel also inhibits synoviocyte proliferation. Paclitaxel is capable of inhibiting proliferation of synoviocytes in vitro and inducing apoptosis (programmed cell death) at concentrations as low as 10⁻⁷ M, and is cytotoxic to the synoviocytes at slightly higher concentrations of 10⁻⁶ to 10⁻⁵ M. Paclitaxel inhibits collagenase production by chondrocytes in vitro, but is not toxic to normal chondrocytes. A concentration of 10⁻⁷ M paclitaxel, for example, reduced collagenase expression by over 50% in cultured chondrocytes stimulated by tumor necrosis factor and interleukin-1. This inhibition occurs downstream from the transcription factor activity of c-fos and c-jun, apparently by disrupting the normal functioning of the AP-1 molecule, resulting in inhibition of transcription of the collagenase gene. As such, inhibition of collagenase secretion by paclitaxel is not strictly due to interruption of the protein secretory pathway, which is dependent upon microtubule function for the movement of secretory granules. Paclitaxel also appears to act at the level of the genetic response to stimuli directing the cell to produce collagenase.

The drug is also known to be effective in treating a number of other indications. Paclitaxel is useful for treating surgical adhesions and post-surgical hyperplasias. In Alzheimer's disease treatment, paclitaxel has been used to stabilize microtubules destabilized by insufficient tau protein levels. U.S. Pat. No. 5,580,898. Paclitaxel is also thought to be effective against polycystic kidney disease (PKD). Sommardahl et al. (1997) Pediatr. Nephrol 11:728-33. Paclitaxel derivatives are also effective in treating psoriasis. EP 747385 and WO 9613494.

Other therapeutic agents have been successfully co-administered with paclitaxel. For example, Vitamin C can be used to increase the efficacy of paclitaxel. Kurbacher et al. (1996) Cancer Lett. 103: 183-189. EP 781552 and EP 787716 describe additional compounds that enhance paclitaxel activity. U.S. Pat. No. 5,565,478 describes combinational therapy of paclitaxel with signal transduction inhibitors for cancer treatment. In treatment of autoimmune arthritis, paclitaxel has been administered with other antiarthritic drugs, such as an angiogenesis inhibitor. U.S. Pat. No. 5,583,153. Anilide derivatives have also been administered to sensitize multidrug-resistant cancer cells to paclitaxel. EP 649410. Paclitaxel can also be administered with antibodies specific to cancerous cells. U.S. Pat. No. 5,489,525. In breast cancer treatment, paclitaxel has been administered in combination with estramustine phosphate. Keren-Rosenberg et al. (1997) Sem. Oncol. 24 (Suppl. 3):S3-26-29. Paclitaxel and IGF-I (Insulin-like growth factor I) have been used together to treat peripheral neuropathy. U.S. Pat. Nos. 5,648,335, 5,569,648 and 5,633,228. Paclitaxel has also been successfully administered along with doxorubicin, cyclophosphamide, and cisplatin. O'Shaughnessy et al. (1995) Breast Cancer Res. Treat. 33:27-37. P-glycoprotein blocker SDZ PSC 833, a cyclosporin derivative, has demonstrated a 10-fold increase in oral bioavailability of paclitaxel in mice. Asperen et al. (1997) Brit. J. Cancer 76:1181-1183. Essential oils have also been suggested to increase paclitaxel's bioavailability. U.S. Pat. No. 5,716,928.

The mechanism of paclitaxel action has been extensively studied and is summarized by Horwitz (1984) Pharm. Ther. 25:83-125. Paclitaxel can act by promoting tubulin assembly into stable aggregated structures which resist depolymerization by dilution, calcium ion, cold, and several microtubule-disrupting drugs. Tubulin depolymerization is essential for cell division, and thus paclitaxel causes this process to cease. Schiff et al. (1979) Nature 277:665-667. Paclitaxel is unique in promoting tubulin polymer formation, whereas other anti-cancer drugs, such as vinblastine and colchicine, prevent this process.

As originally described in Wani et al. [(1971) J. Amer. Chem. Soc. 93:2325-2327], paclitaxel can be purified via alcohol extraction from the Pacific yew tree, Taxus brevifolia. It is also present in other Taxus species, such as T. baccata and T. cuspidata. However, paclitaxel is found only in minute quantities in the bark of these slow-growing trees, causing concern that the limited paclitaxel supply will not meet the demand. Consequently, chemists in recent years have attempted to find alternative or synthetic routes for producing paclitaxel. U.S. Pat. No. 5,019,504 describes the purification of paclitaxel from tissues of T. brevifolia grown in vitro. U.S. Pat. No. 5,322,779 describes the production of paclitaxel from a fungus, Taxomyces andreanae, found in association with the yew tree. More recently, novel compounds have been suggested for use in enhancing plant production of paclitaxel. U.S. Pat. No. 5,710,099.

Paclitaxel has also been synthesized from related compounds found in higher quantities in Taxus trees. These compounds include baccatin III, obtained from Taxus wood, and 10-deacetyl baccatin III, from Taxus leaves. Methods of preparing paclitaxel from these precursor compounds, which themselves lack antitumor activity, have been described. Greene et al. (1988) JACS 110:5917-5919; U.S. Pat. Nos. 5,717,103, 4,857,653, and 4,924,011 (Re. 34,277).

Various synthetic routes and intermediates in paclitaxel synthesis have been described, including a route directed to the synthesis of the tricyclic taxane nucleus from commodity chemicals. Holton et al. (1994) J. Am. Chem. Soc. 116:1597-1598, 1599-1600; Nicolaou et al. (1994) Nature 367:630-634; and Danishefsky et al. (1996) J. Am. Chem Soc. 118:2843-59; and U.S. Pat. Nos. 5,723,635 and 5,726,318. Additional compounds useful in paclitaxel synthesis have also been described. U.S. Pat. No. 5,015,744 describes the use of an oxazinone as a side-chain precursor for paclitaxel synthesis. U.S. Pat. No. 4,876,399 describes an intermediate, 2,5-dihydroxy-2-patchoulene. U.S. Pat. Nos. 5,523,219 and 5,705,671 describe additional intermediates.

Paclitaxel itself has been chemically modified, sometimes producing compounds with even greater antitumor activity than paclitaxel itself. U.S. Pat. No. 4,814,470. Cephalomannine, which differs from paclitaxel and baccatin III in the C-13 ester functionality, demonstrates activity against leukemia in animals. U.S. Pat. No. 4,206,221. Other paclitaxel derivatives include prodrug forms, in which paclitaxel is conjugate to cleavage spacer and sugar groups. EP 781778.

Some paclitaxel derivatives have been produced in attempts to address a significant problem limiting the utility of paclitaxel: paclitaxel is largely insoluble in water. This has created significant problems in developing suitable pharmaceutical formulations for human therapy both in terms of formulation and side effects. The problem is also a serious impediment for experimental research on paclitaxel and its clinical effectiveness. Derivatives of paclitaxel, designed to have increased water solubility, include 2′- and/or 7-position paclitaxel esters, as described in U.S. Pat. No. 4,960,790. Additional substitutions at the C-2′ and C-7 positions were described by Magri et al. (1988) J. Natural Products 51:298-306. 2′-succinyl paclitaxels are described in U.S. Pat. No. 4,942,184; and sulfonated 2′-acryloyltaxol and sulfonated 2′-O-acyl acid paclitaxel derivatives, in U.S. Pat. No. 5,059,699.

Unfortunately, many of these more soluble derivatives reduce paclitaxel antitumor activity. A 2′-succinyltaxol, prepared by the treatment of paclitaxel with succinic anhydride, had decreased in vivo activity compared with paclitaxel, and a 2′-(t-butyldimethylsilyl)taxol was essentially inactive. Magri et al. (1988). Other derivatives, such as 2′-(β-alanyl)taxol, are unstable. Magri et al. (1988). Attempts to derivatize paclitaxel generally increase the molecule's size, which decreases its ability to passively diffuse through the cellular and nuclear membranes of cancerous cells.

The insolubility of paclitaxel itself has yielded a further complication: it has elicited the widespread use of a toxic carrier. Paclitaxel is generally supplied through CTEP (Cancer Therapy Evaluation Program), DCT (Division of Cancer Treatment), and NCI (National Cancer Institute, IND#2280) as a concentrated solution in 50% polyoxyethylated castor oil [Cremophor EL® (BASF)] and 50% dehydrated alcohol. This is then mixed with either a dextrose or sodium chloride solution prior to administration. Although Cremophor EL® is the industry-standard administration vehicle for paclitaxel, Cremophor EL® is itself toxic, causing idiosyncratic histamine release and anaphylactoid-like response. Cremophor EL® is also likely to be the cause of several side effects associated with paclitaxel treatment, including cutaneous flushing, urticaria, dyspnea, bronchospasm, and hypotension. Runowicz et al. (1993) Cancer 71:1591-1596; and Weiss et al. (1990) J. Clin. Oncol. 8:1263-126. In studies with dogs, Cremophor EL® and its fatty acid constituents induced histamine release and hypotension within 10 minutes of administration. Lorenz et al. (1977) Agents Actions 7:63-67. Some tested animals died as a result of this hypotension.

Other organic carriers have been proposed for paclitaxel administration or used in in vitro paclitaxel preparations. Polyethylene glycol (PEG) has been suggested as a substitute emulsifier for paclitaxel, but PEG decreases the antitumor activity of paclitaxel in murine tumor studies. Weiss et al. (1990) J. Clin Oncol. 8:1263-1268. Paclitaxel has also been prepared in solution with dimethylsulfoxide [Kumar et al. (1993) Res. Comm. Chem. Path. Pharm. 80:337-344], which is itself toxic [Kamiya et al. (1967). Nippon Ganka Kiyo 18:387-9; Sperling et al. (1979) Acta Ophthalmol. 57:891-8]. Polysorbate-80 was used in in vitro mixtures containing very low concentrations of docetaxel [Urien et al. (1996) Invest. New Drugs 14:147-151], but polysorbates are toxic, reducing locomotor activity, inducing ataxia and hypotension, and increasing the activity of various carcinogens. Pesce et al. (1989) Ann. Clin. Lab. Sci. 19:70-3; (1984) J. Am. Coll. Toxicol. 3/5:1-82; and Varma et al. (1985) Arzneimittelforschung 35:804-8. Therefore, the sole use of these carriers to solubilize paclitaxel is not a desirable solution to the problem of developing therapeutically effective paclitaxel formulations.

In the absence of workable alternatives, and despite its toxicity, Cremophor EL® remains the standard vehicle used for paclitaxel administration to human patients. Documents demonstrating the universal use of Cremophor EL® in paclitaxel preparations and paclitaxel administration include: Einzig et al. (1991) Cancer Invest. 9:133-136; O'Shaughnessy et al. (1994) Breast Cancer Res. Treat. 33:27-37; Kawano et al. (1994) J. Toxicol. Sci. 19 (suppl. 1):113-122; Asperen et al. (1997) Brit. J. Cancer 76:1181-1183; Sparreboom et al. (1998) Anti-Cancer Drugs 9:1-17; Runowicz et al. (1993) Cancer 71:1591-1596; Sparreboom et al. (1998) Anal. Biochemistry 255:171-175; Plasswilm et al. (1998) Strahlentherapie und Onkologie 174:3742; Xu et al. (1997) Hospital Pharmacy 32:1635-1638; Khan et al. (1997) Ann. Pharmacotherapy 31:1471-1474; Michaud et al. (1997) Ann. Pharmacotherapy 31:1402-1404; Zhang et al. (1997) Anti-Cancer Drugs 8:696-701; Wilson et al. (1997) Ann. Pharmacotherapy 31:873-875; Reinecke et al. (1997) Eur. J. Cancer Part A: United Kingdom 33:1122-1129; Kuangjing Shao et al. (1997) Anal. Chemistry 69:2008-2016; Bonfrer et al. (1997) Tumor Biology; Switzerland 18:232-240; Decorti et al. (1997) Cancer Chemother. Pharmacology 40:363-366; Ho et al. (1997) Neurosurgery 40/6 :1260-1268; Terzis et al. (1997) British J. Cancer 75:1744-1752; Kilbourn et al. (1997) Disease-a-Month 43:282-348; Frasci et al. (1997) J. Clinical Oncology 15:1409-1417; Sharma et al. (1997) International J. Cancer 71:103-107; Georgiadis et al. (1997) Clinical Cancer Research 3:449-454; Zhang et al. (1997) Cancer Chemother. Pharmacology 40:81-86; EP 694303; WO 94/12031; and U.S. Pat. Nos. 5,733,888, 5,731,334, 5,719,265, 5,714,512, 5,703,117, 5,698,582, 5,696,153, 5,686,488, 5,683,715, 5,681,846, 5,670,537, 5,665,761, 5,648,335, 5,648,090, 5,641,803, 5,633,228, 5,621,001, 5,616,608, 5,616,330, 5,614,549, 5,608,087, 5,604,202, 5,569,648, 5,583,153, 5,580,899, 5,569,720, 5,565,478, 5,504,102, 5,496,846, 5,496,804, 5,478,860, 5,403,858.

Numerous attempts have been made to produce aqueous solutions of hydrophobic drugs. For instance, formulations of cisplatin combined with dextran, polyglutaric acid, DNA, proteins, hyaluronic acid, etc. were compared. It was found that many of these excipients were unacceptable as they bound the drug too tightly and did not release it on administration or did not bind enough drug to produce a pharmaceutically acceptable formulation. DNA was in the category of excipients which bound too tightly. Proteins, including serum albumin, were found to bind limited amounts of drug, only a portion of which was reversibly bound.

Albumins have been used as excipients as bulk stabilizers for a number of drug formulations, particularly biologicals such as interleukins and cytokines. Human serum albumin is a large component of interleukin-4 preparations. Meyer et al. (1994) Pharm. Res. 11: 1492-1495. Albumin has also been conjugated to drugs to increase uptake of the drug and derivatized albumins have been used to couple drugs and enhance uptake through the blood-brain barrier. Sinn et al. (1990) Nucl. Med. Biol. 17:819-827; Pardridge et al. (1990) J. Pharmacol Exp. Ther. 255:893-899; Flume et al. (1989) Pharm. Acta Helv. 64:351-352; and JP 61001622. WO 94/01090 describes broad formulations of hydrophilic peptides and “sparingly water soluble” active compounds. Albumin is a cost-limiting component for use in drug stabilization. Thus, unless an unstable drug can be stabilized in some other fashion, albumin is not ideal as a bulk stabilizing agent. Further, native albumin is being phased out of use as it may contain infectious agents such as prions. Replacement with recombinant albumin may result in an even more costly product. Therefore, in order to produce a commercially available, pharmaceutically acceptable albumin-bound drug, the drug must be bound reversibly to the albumin in a high molar ratio.

The need remains for aqueous pharmaceutically acceptable formulations of paclitaxel which are easy and inexpensive to prepare, produce fewer side effects, and in which the drug retains high water solubility and activity.

### SUMMARY AND OBJECTS OF THE INVENTION

In one embodiment, the invention provides an optically clear, pharmaceutically acceptable aqueous composition comprising paclitaxel or a derivative thereof, serum albumin or a fragment thereof, and a pharmaceutically acceptable vehicle. In various embodiments, the composition comprises no more than 10% organic solvent, and has a pH of about 3.0 to about 4.8 (the pI of albumin). In various embodiments, the composition comprises about 1 to about 10%, about 2 to about 8%, or about 4 to about 6% v/v (volume/volume) organic solvent. In a preferred embodiment, the composition is essential free of organic solvent. The organic solvent is preferably an alcohol, most preferably ethanol. In various embodiments, the pH is about 3.0 to about 4.8, about 4.0 or less, about 3.0 to about 4.0, or about 3.4 to about 3.8. In various embodiments, the ratio of paclitaxel or derivative thereof to albumin is at least about 1:5, at least about 1:4, at least about 1:2, at least about 1:1, or at least about 2:1. In various embodiments, the serum albumin is defatted, undefatted or a mixture of defatted and undefatted forms. In various embodiments, the serum albumin is mammalian, preferably human. In various embodiments, the serum albumin is at least about 50%, at least about 60%, at least about 70%, at least about 80%, or at least about 90% monomeric. In various embodiments, at least about 70%, at least about 80%, at least about 85%, or at least about 90% of the paclitaxel or derivative thereof is bound to albumin. In another embodiment, the composition is lyophilized. In another embodiment, the composition is reconstituted from a lyophilized formulation. In various embodiments, the concentration of paclitaxel is greater than about 25 μg/ml, greater than about 50 μg/ml, greater than about 100 μg/ml, greater than about 200 μg/ml, greater than about 300 μg/ml, greater than about 400 μg/ml, or greater than about 500 μg/ml. In another embodiment, the composition is coated onto an implantable device such as a stent or wrap. In some embodiments, the device is catheter-based and/or used in conjunction with surgery. In some embodiments, the coating prevents restenosis, local tumor growth or tissue over-growth and/or chronic inflammation.

The composition is characterized by having optical clarity for a length of time sufficient to administer to a patient or to process further (e.g., subject to drying). In another embodiment, the composition is optionally dried and stored as a dried “storage-stable” composition. The dried preparation of the composition is resolubilized prior to administration. In a preferred embodiment, the drying process is lyophilization. In one embodiment, the composition prior to drying comprises McIlvaine buffer. In another embodiment, the lyophilized preparation of the composition is optionally reconstituted with a physiologically acceptable vehicle, such as McIlvaine buffer, water, a sugar solution such as dextrose or glucose, or certain saline solutions, including dilutions of saline. The reconstituted compositions can be essentially free of solvent, which can be removed in the lyophilization step. The resolubilized composition can be 2-10 times more concentrated than the original pharmaceutically acceptable composition, depending on the concentration of paclitaxel in the pre-lyophilized composition. Thus, the invention encompasses a resolubilized composition which is optically clear for at least 8 hours after reconstitution. The composition comprises less than 10% organic solvent and has a pH of about 3.0 to about 4.8 upon reconstitution, at least about 70% of the paclitaxel introduced into the composition is bound to the serum albumin, and the paclitaxel concentration in the composition is at least 50 μg/ml. The invention further encompasses methods of administration of the reconstituted composition wherein a therapeutically effective amount of paclitaxel can be administered as a 1 to 3 hour (or greater) injection or as a bolus.

In another embodiment, the invention encompasses a method of treatment, comprising administering to a patient a therapeutically effective amount of an optically clear, pharmaceutically acceptable aqueous composition comprising paclitaxel or a derivative thereof, serum albumin and a pharmaceutically acceptable vehicle, as described above. The indication to be treated with the composition can include any indication known in the art to be treatable with paclitaxel, including, but not limited to, cancer. Preferably, the cancer affects cells of the bladder, blood, bone, brain, breast, cervix, colon, epithelium, digestive tract, head/neck, kidneys, liver, lung, mouth, ovaries, pancreas, prostate gland, skin, stomach, testicles, or tongue. The indication can also include, but is not limited to, paclitaxel-treatable indications such as Alzheimer's disease, kidney disease, peripheral neuropathy, psoriasis, restenosis, rheumatoid arthritis, systemic lupus erythematosus, surgical adhesions, or tissue overgrowth after surgery. Preferably, the patient is a mammal. More preferably, the mammal is a human.

The composition and methods of use thereof can optionally further comprise an additional biologically active ingredient, including but not limited to those known to function synergistically with paclitaxel. In various embodiments, the additional agent includes, but is not limited to, G-CSF (granulocyte colony-stimulating factor), GM-CSF (granulocyte macrophage colony-stimulating factor), IL-4 (interleukin 4), IGF-I, analide derivatives, antiarthritics (e.g., an angiogenesis inhibitor), antibodies specific to cancer cells, antineoplastics (e.g., carboplatin, cyclophosphamide, estramustine phosphate, and etoposide), doxorubicin, immunosuppressants (e.g., cisplatin and cyclophosphamide), steroidal and non-steroidal hormone (e.g., cortisone), transduction inhibitors, and vitamins (e.g., vitamin C). The composition can further comprise low concentrations of excipients such as polyethylene glycol, detergents, organic solvents, or organic or inorganic acids.

In another embodiment, the invention encompasses a method of making an optically clear, pharmaceutically acceptable aqueous composition comprising paclitaxel or a derivative thereof, serum albumin and a pharmaceutically acceptable vehicle, as described above, comprising the steps of preparing a solution of the paclitaxel or a derivative thereof, preparing a solution of serum albumin, and slowly combining the solutions. Due to stable binding of Ptx to serum albumin, the rate of addition of the Ptx solution to the albumin solution can be decreased to assist in more optimal loading of Ptx onto albumin. The paclitaxel solution can, for example, be added dropwise at a controlled rate; this rate can be, for example, at about 0.1 to 10 ml/min, e.g., 1 ml/min or slower, and the drop size can be 8 to 20 μl. In various embodiments, the ratio of paclitaxel or derivative thereof to albumin is at least about 1:1 or at least about 2:1, and the solutions are combined at a temperature below room temperature, about 2° C. to 8° C., or about 4° C. In various embodiments, the ratio of paclitaxel or derivative thereof to albumin is at least about 1:5, at least about 1:4, at least about 1:2, at least about 1:1, or at least about 2:1. It is anticipated that ratios of 3:1 and possibly even 4:1 can be achieved according to the invention described herein, by controlling the rate of addition of the paclitaxel to the albumin solution to a degree that does not interfere with continued stability during processing.

Preferably the paclitaxel is “optimally concentrated.” This term means that the paclitaxel concentration in the composition allows a solvent concentration of 1-10% v/v. The molar ratio of paclitaxel:albumin and the final concentration of paclitaxel in the albumin solution are optimized, such that the paclitaxel remains in solution for a length of time practical for administration or lyophilization/reconstitution. We have found that the highest concentrations of paclitaxel and optimal molar ratios are achieved with final ethanol concentrations in the 1-10% range, more preferably in the 2-8% range, most preferably about 4-6%. This results in the smallest volumes for administration or lyophilization/reconstitution, which enables more rapid administration, if desired. When the composition is dried and reconstituted, the solvent can be removed during the drying, and the reconstituted formulation can be essentially free of solvent (e.g., comprising preferably less than about 1%, more preferably less than about 0.5%, or most preferably less than about 0.1% v/v solvent).

The foregoing methodology may empirically be determined to extend to other water insoluble drugs and globulins (albumin substitutes).

### BRIEF DESCRIPTION OF THE FIGURES

FIG. 1 depicts the effect of ethanol concentration on the solubilization of a fixed concentration of paclitaxel in the presence of human serum albumin (HSA) added at different molar ratios.

FIG. 2 depicts the effect of molar ratio on the solubilization of increasing paclitaxel amount to a fixed amount of HSA in a neutral pH, aqueous 4% ethanol (ETOH) solution.

FIG. 3 depicts the effect of ethanol concentration on the solubilization of paclitaxel at different concentrations in the presence of HSA added to molar ratios of 1:1 and 1:2.

FIG. 4A-4D depicts the effect of pH, time and tube material on binding of paclitaxel to non-defatted HSA. 4A, Turbidity after 24 hr incubation in glass tubes. 4B, Turbidity after 96 hr incubation in glass tubes. 4C, Turbidity after 24 hr incubation in plastic tubes. 4D, Turbidity after 96 hr incubation in plastic tubes. All incubations were performed at 23° C.

FIGS. 5A and 5B depict the effects of various formulations on resolubilization of compositions of paclitaxel and serum albumin. 5A, Turbidity measurement of resolubilized Ptx-HSA preparations of 200 μg/ml Ptx following a 0.5-hr or 17-hr incubation. 5B, Turbidity measurement of resolubilized Ptx-HSA preparations of 50 μg/ml Ptx following a 0.5-hr or 17-hr incubation.

FIG. 6 depicts the effect of different preparations of HSA on the binding of paclitaxel (200 μg/ml) to HSA at a molar ratio of 1:1 in saline solutions of different ionic strengths containing 5% ethanol.

FIGS. 7A and B depict the effect of pH and paclitaxel concentration on the binding of paclitaxel to HSA at a molar ratio of 1:1.

FIGS. 8A and B depict the effect of different preparations of HSA and saline strength on the turbidity of paclitaxel solutions containing HSA at a molar ratio of 1:1.

FIG. 9 depicts the effect of ethanol concentration on the recovery and binding of Ptx to undefatted and defatted HSA at pH 3.5 and 7.

FIG. 10 depicts the pH profile for recovery and binding of Ptx to defatted and undefatted HSA and the stability of the resulting Ptx:HSA formulations.

FIG. 11A depicts the effect of molar ratio on the recovery and binding of Ptx to undefatted and defatted HSA at pH 3 and 7 in 4% ethanol.

FIG. 11B depicts the effect of the molar ratio on the recovery and binding of Ptx to undefatted and defatted HSA at pH 3 and 7.

FIG. 12 depicts the combined effect of salt and ethanol concentration on the stability of Ptx:HSA formulations at 1:1 and 1:2 molar ratios, using acid-defatted HSA.

FIG. 13 depicts the stability after 24 hour storage of lyophilized formulations of Ptx:HSA, in an pre-lyophilization volume of 3 ml, reconstituted in 3 or 6 ml.

FIG. 14A depicts the effect of microfiltration on the recovery of the acidic liquid Ptx/HSA formulation. A: Analysis of filter saturation. 2 mL of the formulation was passed through the same filter 3 times (filtration 1 to 3) and recovery of Ptx analyzed each time in the filtrate. B: Analysis of filter binding capacity: 3-mL of the formulation was passed through different filters by removing the filtrate from filter #1 and passing it through filter # 2 and so on for a total of 3 filters. Uncentrifuged is the starting formulation mixture containing precipitable and soluble Ptx. Centrifuged is same as for the standard analysis of total soluble Ptx (HSA-bound and free). Microfiltration was through 0.2 micron nylon mesh or SFCA (surfactant-free) filter

FIG. 14B depicts the effect of microfiltration on the recovery of the neutral pH and acidic liquid Ptx/HSA formulations. Analysis of filter saturation. 2 mL of the formulation was passed through the same filter 3 times (filtration 1 to 3) and recovery of Ptx analyzed each time in the filtrate. Uncentrifuged is the starting formulation mixture containing precipitable and soluble Ptx. Centrifuged is same as for the standard analysis of total soluble Ptx (HSA-bound and free).

FIG. 15A depicts the effects of antioxidants on HSA dimerization at acidic pH.

FIG. 15B depicts the effects of antioxidants on reconstituted lyophilized formulations.

### MODES FOR CARRYING OUT THE INVENTION

It would be highly advantageous to the therapy of a number of indications, including cancer, to obtain a pharmaceutical formulation comprising an optically clear aqueous solution of paclitaxel. The present invention encompasses a method of making an optically clear, pharmaceutically effective, aqueous composition of paclitaxel, a serum albumin, and a physiologically acceptable vehicle, compositions obtained thereby and methods of use thereof. The standard vehicle for paclitaxel delivery comprises Cremophor EL® (polyoxyethylated castor oil). The present invention circumvents the use of this toxic vehicle.

The serum albumin for use in the present invention is preferably natural, more preferably mammalian, more preferably human, more preferably recombinant human serum albumin. The paclitaxel is preferably non-covalently bound to the serum albumin. The serum albumin is predominantly (at least about 50%, at least about 60%, at least about 70%, at least about 80%, or at least about 90%) monomeric. Although the albumin is preferably monomeric, it can typically contain up to about 15% dimeric protein. The serum albumin can be de-fatted, or be fatted (containing fat). The serum albumin can have a complement of fat similar to that of serum albumin as found in the human body (about 1 to about 3 moles fatty acid per mole of serum albumin), or it can have a different complement of fat. Preferably, the serum albumin is defatted. The albumin is also preferably recombinant. In the case of recombinant albumin, the fat content may differ from that of native albumin.

The composition can also contain an organic solvent. The organic solvent can be any known in the art, including, but not limited to, an alcohol, an aromatic compound, a detergent, an ether, a fat, a fatty acid, a triglyceride of a fatty acid, a glycol, a halogenated compound, lecithin, an oil, DMSO, or any combination of these solvents. Preferably, the organic solvent is an alcohol. Even more preferably, the alcohol is ethanol. Preferably, the final concentration of ethanol (in either the original or reconstituted solution) in about 1-10% v/v and more preferably about 2-8% v/v and most preferably about 4-6% v/v.

In one embodiment, the composition comprising paclitaxel, a serum albumin and an organic solvent is dried to form a storage-stable composition, stored as a dried composition (e.g., a lyophilized preparation), and then resolubilized with a vehicle prior to administration. Preferably, the composition comprises less than 10% organic solvent and has a pH of about 3.0 to about 4.8 upon reconstitution, at least about 70% of the paclitaxel introduced into the composition is bound to the serum albumin, and the paclitaxel concentration in the composition is at least 50 μg/ml. In a preferred embodiment, the drying process is lyophilization. In one embodiment, the composition prior to drying comprises McIlvaine buffer. Dawson et al. (1986) Data for Biochemical Research, 3rd ed., Oxford Science Publ., p. 427. In another embodiment, the composition is reconstituted after lyophilization with a physiologically acceptable vehicle, such as McIlvaine buffer, a sugar solution such as dextrose or glucose, water, or certain saline solutions including dilutions of saline, so as to attain a pharmaceutically acceptable vehicle upon reconstitution. In another embodiment, the composition comprising paclitaxel, a serum albumin and a physiologically acceptable vehicle can be coated onto an implantable device such as a stent or wrap. In some embodiments, the device is catheter-based and/or used in conjunction with surgery. In some embodiments, the coating prevents restenosis, local tumor growth or tissue over-growth and/or chronic inflammation.

Preferably, the amounts of paclitaxel, serum albumin, solvent, and ratios between these ingredients, and pH are such that the composition is optically clear, indicating that none of the components has precipitated or formed crystals. The serum albumin is present in appropriate amount of solvent so that the final balance between precipitation of paclitaxel from solution and binding of paclitaxel to albumin favor binding of paclitaxel to albumin. Under the conditions described herein, we have found that paclitaxel-albumin binding is quite stable as evidenced by the ability to obtain concentrated optically clear solutions upon reconstitution of the storage-stable composition. Under the conditions described herein, the paclitaxel is said to be “deeply embedded” in the albumin.

The paclitaxel is preferably present at a concentration at which it remains in solution when bound to the serum albumin, such as a concentration of greater than about 25 μg/ml, greater than about 50 μg/ml, greater than about 100 μg/ml, greater than about 200 μg/ml, greater than about 300 μg/ml, greater than about 400 μg/ml, or greater than about 500 μg/ml, in a ratio about 1:5 (or greater) paclitaxel to albumin, and preferably in a ratio 1:4 or greater, more preferably 1:2 or greater, even more preferably 1:1 or greater, and most preferably 2:1 or greater. Preferably, the ratios of organic solvent and paclitaxel in the formulation are such that the paclitaxel remains in solution, such as a formulation comprising about 2 to about 10% ethanol, preferably about 4-8% ethanol, and greater than about 50 μg/ml paclitaxel, greater than about 100 μg/ml, greater than about 200 μg/ml, greater than about 300 μg/ml, greater than about 400 μg/ml, or greater than about 500 μg/ml with a molar excess of albumin. We have found that decreasing the organic solvent from 20% to about 4% final volume increases binding of paclitaxel to serum albumin. Most preferably, the organic solvent is about 4 to about 6% of the final volume. Preferably, the amounts of albumin and solvent are such that the albumin remains in solution, such as a formulation comprising about 4% to about 10% ethanol, about 4-230 mg/ml albumin and about 50-600 μg/ml paclitaxel, preferably 200-400 μg/ml. Preferably, the molar ratios of paclitaxel:albumin, paclitaxel:ethanol and albumin:solvent are such that paclitaxel and albumin remain in solution, such as about 1:4 to about 2:1 (paclitaxel:albumin) at a fixed concentration of 50, 100, 200, 300, 400, 500, 600 or 1000 μg/ml paclitaxel, 4-8% ethanol and a pH of 3-4.8. The serum albumin can be defatted or fatted, the state being appropriate to maximize solubility of paclitaxel, such as defatted serum albumin in about a 1:1 molar ratio with about 100 μg/ml paclitaxel in about 4% ethanol at pH 3-4.8. Preferably, the serum albumin is defatted by lowering the pH to about 3.4 to 3.8.

Preferably, the pH of the composition is such that the paclitaxel and albumin remain in solution and the paclitaxel binds noncovalently to the albumin. Typically, the optimal pH is at or below the pI of the albumin. For instance, a pH of about 4.8 or lower is optimal for a solution of about 50, about 100, about 200, about 300, about 400, 500, or about 600 μg/ml paclitaxel at an approximately 1:4 molar ratio with serum albumin in about 4% ethanol; or a pH of about 3.0 to about 4.8 for a composition of up to about 600 μg/ml paclitaxel at 1:2 molar ratio with serum albumin in about 5% ethanol. Based on the present disclosure, additional amounts and ratios which result in optically clear formulations can be readily determined by experimentally mixing the ingredients in various quantities at different rates.

The use of serum albumin and organic solvents to solubilize bioactive agents is provided by U.S. Pat. Nos. 4,842,856 and 5,051,406. These patents provide only very broad ranges of drug to albumin. As shown herein, only a very few combinations and narrow ranges of pH, amounts and ratios of paclitaxel, albumin and solvent are suitable for producing compositions of paclitaxel that are commercially and clinically efficacious. Without wishing to be bound by any one theory, it may be that some formulations can solubilize otherwise water-insoluble drugs such as paclitaxel, because a ratio of ingredients has been achieved such that the drug leaves the aqueous phase of the formulation and preferentially binds to the albumin. This binding may be due to phenomena similar to the so-called hydrophobic effect theory. This theory states that when two dissolved molecules unite to form a complex, the two cavities containing the separated species coalesce into a single cavity holding the complex. Thus, the invention is directed to stable reversible binding. Compositions comprising high concentrations of organic solvents, such as those suggested by the patents cited above, can, at the desired concentration of drug, result in unworkable, optically unclear formulations, indicating that unacceptable levels of precipitation or crystal formation or the like occurred.

Preferably, the formulation of the present invention is optically clear. Clarity is determined experimentally for the duration of the time from preparation of the formulation to administration. Because clarity can decrease with both time and paclitaxel concentration, formulations prepared for immediate administration can comprise higher concentrations of paclitaxel than formulations which will undergo prolonged storage prior to administration. Preferably, the solvent and paclitaxel are present at concentrations at which paclitaxel remains in solution for at least 24 hr, such as a concentration of greater than about 25 μg/ml or greater than about 50, greater than about 100, greater than about 200, greater than about 300, greater than about 400, greater than about 500, greater than about 600 μg/ml or greater than about 1000 μg/ml. Preferably, the amount of organic solvent is such that the paclitaxel and serum albumin remain in solution, for instance, a concentration of about 2% to about 10% ethanol in a solution comprising up to about 500 μg/ml solution of paclitaxel, or a concentration of about 4% to about 8% ethanol in a solution comprising up to about 250 mg/ml albumin. Preferably, the ratios of paclitaxel:solvent, albumin:solvent, and paclitaxel:albumin are such that the paclitaxel and albumin remain in solution, such as molar ratios of about 1:4 to about 2:1 (paclitaxel:albumin) with fixed concentrations of about 50, about 100, about 200, about 300, about 400, or about 500 μg/ml paclitaxel and about 5% ethanol. However, based on the present disclosure, additional amounts and ratios of ingredients that result in acceptably optically clear formulations can be readily determined by mixing the ingredients in varied amounts and ratios and testing for cloudiness. The method of the present invention allows for binding of substantially all of the paclitaxel to a commercially efficacious amount of albumin in a volume appropriate for administration to a patient, for subsequent processing to form a dried storage-stable composition and for reconstitution at a commercially practicable volume and physiologically acceptable pH. Preferably, for drying by lyophilization, the volume is not more than 100 ml and is preferably less than 50 ml with a total of about 30 mg paclitaxel.

Preferably, the solubility attained is commercially appropriate (in terms of required albumin, bound paclitaxel and conditions of reconstitution, including pH, volume, and salt concentration, giving an optically clear solution for the requisite time period) for a range of paclitaxel concentrations required for the desired dosage regiment, when translated into dosage volume. Preferably the volume is such that the dosage can be administered in a bolus.

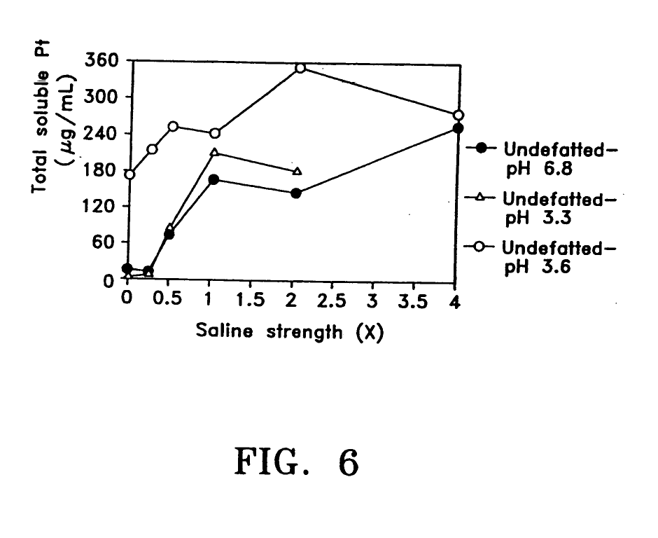

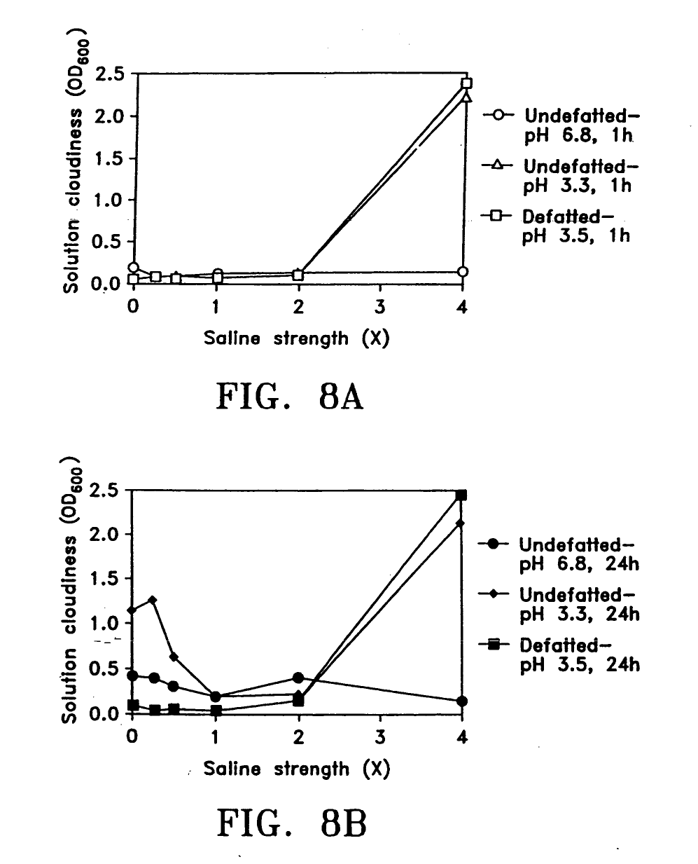

The final volume of the composition is a function of the salt concentration. Preferably, the salt concentration is isotonic. We have found that it is possible to obtain an optically clear composition of a high concentration of paclitaxel by using defatted albumin at low salt concentration. This is in contrast to the use of fatted albumin which requires normal saline or higher salt concentration to achieve an optically clear composition of a high concentration of paclitaxel. This illustrated in FIGS. 6 and 8. This impacts positively on the final volume of the reconstituted composition. The availability of formulations of different ionic strengths allows manufacture of low ionic strength formulations for patients who require reduced intake of ions such as potassium and sodium.

The serum albumin can be defatted or fatted, preferably defatted. Preferably, the serum albumin is recombinant and has a lower fat content than commercially available native serum albumin. More preferably, the serum albumin is recombinant and defatted. By “defatted” is meant that the fat has been at least partially removed from the serum albumin. Methods of defatting (e.g., by acidification) are known in the art. In a preferred embodiment, the fat is not only removed from the albumin but also removed from the albumin-containing solution (e.g., by dialysis or filtration through carbon-impregnated filter media). By “nondefatted”, “undefatted” or “fatted” is meant that the albumin retains at least some fat. Fatted albumin has at least 1 to 3 moles of fat per mole of albumin. Defatted albumin has less than 1 mole of fat per mole of albumin, preferably less than 0.5, and more preferably less than 0.25. Most preferably, defatted serum albumin is essentially free of fat.

Preferably, the pH of the composition is such that paclitaxel and albumin remain in solution, typically at or below the pI of the albumin. For instance, a pH of about 4.8 or lower is effective for a solution of about 50, about 100, about 200, or about 300 μg/ml paclitaxel at an approximately 1:1 molar ratio with serum albumin in about 5% ethanol; or a pH of about 3.0 to about 4.8 for a composition of up to about 500 μg/ml paclitaxel at an approximately 1:4, and preferably 1:1, molar ratio with serum albumin in about 5% ethanol. The present invention allows binding of a high concentration of paclitaxel to albumin by incubating the albumin at or below its pI.

The invention further encompasses compositions containing at least one additional active agent. In various embodiments, the additional agent includes, but is not limited to, G-CSF, GM-CSF, IL-4, IGF-I, analide derivatives, antiarthritics, antibodies specific to cancer cells, antineoplastics (e.g., carboplatin, cyclophosphamide, estramustine phosphate, and etoposide), doxombicin, immunosuppressants (e.g., cisplatin and cyclophosphamide), steroidal and non-steroidal hormone (e.g., cortisone), transduction inhibitors, and vitamins (e.g., vitamin C).

By “paclitaxel” (“Ptx”) is meant any taxane or related compound, including paclitaxel or any analog, prodrug or derivative thereof, typified by, but not limited to, the diterpene compound identified and structurally described by Wani et al. (1971). As used herein, therefor, “paclitaxel” includes, but is not limited to, any taxane, taxoid, taxanoid, or taxan, and analogs and derivatives thereof, and is preferably (2aR-(2aα,4β,4aβ,6β, 9α(αR*,βS*), 11α, 12α, 12aα, 12bα))-β-Benzoylamino)-α-hydroxybenzenepropanoic acid 6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca(3,4)benz(1,2-b)oxet-9-yl ester [sold under the brand name TAXOL™ by Bristol Myers-Squibb; other designations: Paclitaxel, CAS Registry No. 33069-624, ANZATAC (Faulding), PANXENE (Ivax)]. Other paclitaxels include, but are not limited to, Docetaxel, (2aR-(2aα,4β,4aβ,6β,9α,(αR*,βS*),11α,12α,12aα,12bα))-β-(((1,1-Dimethylethoxy)carbonyl)amino)-α-hydroxybenzenepropanoic acid 12b-(acetyloxy)-12-(benzoyloxy)-2a,3,4,4a,5,6,9,10,11,12,12a,12b-dodecahydro-4,6,11-trihydroxy-4a,8,13,13-tetramethyl-5-oxo-7,11-methano-1H-cyclodeca(3,4)benz(1,2-b)oxet-9-yl ester [Rhone-Poulenc Sante; other designations: N-debenzoyl-N-(tert-butoxycarbonyl)-10-deacetyltaxol, CAS Registry No. 114977-28-5, Drug Codes NSC-628503 and RP-56976, TAXOTERE (Rhone-Poulenc Sante)].

Additionally, paclitaxel analogs and derivatives further include, but are not limited to, compounds such as baccatin III, 10-deacetylbaccatin III, 2′-(triethylsilyl)taxol; 7-epitaxol; 2-debenzylisotaxol; 2′(N-benzoylcarbamyl)-β-alanyl)-7-oxo-5,6-dehydro-5-O-secotaxol; 20-acetoxy-4-deacetyl-5-epi-20,O-secotaxol; and 7-(triethylsilyl)-baccatin III. Taxane analogs, prodrugs and derivatives are described in, inter alia, Leu et al. (1993) Cancer Res. 53:1388-1391; U.S. Pat. Nos. 4,206,221; 4,814,470; 4,857,653; 4,876,399; 4,942,184; 4,960,790; 5,059,699; 5,703,247; 5,705,508; 5,710,287; 5,714,513; 5,717,103; 5,719,177; 5,721,268; 5,726,318; 5,726,346; 5,728,725; 5,728,850; and EP 781778. Examples of manufacturing paclitaxel and derivatives thereof can be found in U.S. Pat. Nos. 4,960,790 and 4,814,470; such examples can be followed to formulate the paclitaxel for use in this invention.

In one aspect, the present invention relates to the use of serum albumin and organic solvents to solubilize paclitaxel and water-insoluble derivatives thereof. However, when used in compositions of the present invention, some paclitaxel derivatives and analogs which are more water-soluble than paclitaxel may require less organic solvent (e.g., alcohol) and/or serum albumin to solubilize than paclitaxel.

Pharmaceutically acceptable, optically clear formulations of paclitaxel can be derived based on the disclosure herein. For example, a solution of serum albumin can be prepared (and is commercially available as, for example, a 20% solution). This can be combined with solutions of increasing concentrations of the paclitaxel. Optimal parameters to obtain the desired paclitaxel concentration include modifying the concentration of serum albumin, and keeping the pH, at or below the pI of the albumin, speed of addition of paclitaxel to serum albumin, concentration of organic solvent, salt concentration, temperature and incubation time. These can also be readily determined based on the disclosure herein, using, for example, this disclosure as suggested initial test conditions.

By “cancer” is meant the abnormal presence of cells which exhibit relatively autonomous growth, so that they exhibit an aberrant growth phenotype characterized by a significant loss of cell proliferation control. Cancerous cells can be benign or malignant Cancer types include, but are not limited to, those affecting cells of the bladder, blood, bone, brain, breast, cervix, colon, epithelium, digestive tract, head/neck, kidneys, liver, lung, mouth, ovaries, pancreas, prostate gland, skin, stomach, testicles, or tongue.

By a “patient” is meant an individual under surgical or medical treatment or supervision, including those individuals suffering from an indication such as cancer and persons suspected of having or genetically predisposed to have such an indication. The individual is preferably a mammal, more preferably a human being.

By “pharmaceutically acceptable” is meant a composition suitable for use in treatment of humans and/or animals. Typically, the formulations are relatively non-toxic and do not cause additional side effects compared to the drug delivered. In the case of chemotherapeutics which are generally toxic, a pharmaceutically acceptable formulation is one which delivers an amount of drug sufficient to kill tumor cells and sparing the patient although there maybe side effects inherent to the drug.

By a “therapeutically effective amount” is meant an amount effective to achieve a desired and/or beneficial effect. An effective amount can be administered in one or more administrations. For purposes of this invention, a therapeutically effective amount is an amount appropriate to treat an indication such as cancer. By treating an indication is meant achieving any desirable effect, such as the ability to palliate, ameliorate, stabilize, reverse, slow or delay disease progression, increase the quality of life, and/or to prolong life. Such achievement can be measured by any method known in the art, such as physical measurement of tumor size, monitoring of the level of cancerous antigens in blood serum, or measuring patient life.

By “globulin” is meant proteins obtained in fractions II-V of serum, the “Cohn fractions.” Such proteins are separated on the basis of pI and include serum albumin. Typically, globulins are globular proteins with a hydrophobic center. The term “globulin” includes serum albumin.

A “serum albumin,” as the term is used herein, can be natural or recombinant serum albumin and/or a serum albumin fragment. The serum albumin should be non-toxic and non-immunogenic. Preferably the serum albumin is natural (e.g., comprising a full-length amino acid sequence found in nature), more preferably a mammalian serum albumin, more preferably a human serum albumin, even more preferably a recombinant human serum albumin, and even more preferably, a primarily (at least about 80%) monomeric recombinant human serum albumin. This albumin can be modified by, for example, attachment or removal of fatty acids, lipids, or portions of other proteins. For example, serum albumin can be defatted or non-defatted (e.g., containing about 1 to about 3 moles fatty acid per mole of serum albumin), or defatted to which appropriate fatty acids are covalently or non-covalently attached. Preferably, the albumin is defatted. Some commercially available serum albumin derived from serum has 1 mole of fats per mole of serum albumin. The albumin can contain deletions, substitutions, and/or additions in amino-acid sequence from the naturally-occurring sequence. Deletions are exemplified by biologically active fragments of serum albumin, such as those containing only serum albumin subdomains IIA and IIIA, such as those disclosed in U.S. Pat. No. 5,780,594. Preferably the serum albumin is “natural,” e.g., comprising a full-length amino acid sequence as found in nature. The serum albumin can also include fragments of serum albumin, which can be produced recombinantly or by mechanical, chemical or proteolytic cleavage. Preferably, the serum albumin is mammalian or avian. The mammalian serum albumin can include, but is not limited to, human, bovine, rat, mouse, equine, porcine, ovine and guinea pig serum albumin. The avian albumin can include, but is not limited to, ovalbumin. As used herein, the term “serum albumin” encompasses all albumins, even if not normally present in blood. Even more preferably, the serum albumin is human serum albumin (HSA). The serum albumin is preferably non-aggregated or loosely aggregated; and predominantly (greater than 80%) monomeric. Preferably, at least about 50%, at least about 60%, at least about 70%, at least about 80%, or at least about 90% monomeric of the serum albumin is monomeric. The serum albumin can be bonded to a synthetic polymer (polyalkylene glycols, such as linear or branched chain polyethylene glycol), polyvinyl alcohol, polyhydroxyethyl methacrylate, polyacrylic acid, polyethyloxazoline, polyacrylamide, polyvinyl pyrrolidinone, and the like), phospholipids (such as phosphatidylcholine (PC), phosphatidylethanolamine (PE), phosphatidylinositol (PI), sphingomyelin, and the like), proteins (such as enzymes, antibodies, and the like), polysaccharides (such as starch, cellulose, dextrans, alginates, chitosan, pectin, hyaluronic acid, and the like), or chemical modifying agents (such as pyridoxal 5′-phosphate, derivatives of pyridoxal, dialdehydes, diaspirin esters, and the like), or combinations of any two or more thereof.

Human serum albumin is available from Armour Pharmaceutical Div., Rhone-Poulenc. Rorer, Collegeville, Pa., and Fluka Chemika-BioChemika, Buchs, Switzerland. rHSA (recombinant human serum albumin) can be prepared, for example, by use of recombinant techniques such as described in EP 0 683 233 and is commercially available from Delta Biotechnology Ltd., Nottingham NG71 FD, Great Britain. Additional methods of purifying human serum albumin are described in, inter alia, U.S. Pat. Nos. 5,710,253; 5,656,729; 4,228,154; 4,216,205; and 2,765,299. Production of recombinant HSA is described, inter alia, in U.S. Pat. Nos. 5,691,451; 5,612,197; 5,521,287; 5,503,993; 5,440,018; 5,334,512; and 5,260,202.

Albumin can be in the normal form, or in the fast form induced at a pH below 4.0, or in the expanded form induced at a pH below 3.5. Albumin is known to undergo major reversible conformational isomerization with changes in pH. Foster (1977) in Albumin Structure, Function and Uses (Rosenoer et al., eds.), pp. 53-84; Luetscher (1939) J. Am. Chem. Soc. 61:2888. The interaction of albumin with fatty acids also induces major conformational changes. Peters (1985) Adv. Protein Chem. 37:161-245. There are 5 pH-induced albumin forms:

| N | Normal | at neutral pH |
|---|---|---|
| B | Basic | at >pH 8.0 |
| F | Fast | at <pH 4.0 |
| E | Expanded | at pH <3.5 |
| A | Aged | during prolonged storage at pH >8.0 |

N-F transition occurs abruptly at pH <4.0 and involves the unfolding of domain III from the rest of the molecule. The C-terminal half, or tail, dissociates from the “head” of the albumin, a process reversed near neutral pH. King (1973) Arch. Biochem. Biophys. 156:509-520. The F-form is characterized by a dramatic increase in viscosity, much lower solubility, predominantly heart-shaped conformation, and a significant loss in helical content. Structurally, the interface between the two halves of the molecule are held together by both hydrophobic and salt bridge interactions. Hydrophobic interactions associate IA, IB, and IIA, with IIB, IIIA, and IIIB, and involve an interdomain cluster of hydrophobic amino acids (Phe, Leu, Ala, Trp, Val, and Tyr). The N-F transition and the pH of the transition are highly conserved, implying a physiological role of this conformation. Carter et al. (1994) Adv. Protein Chem. 45:153-203. It has now been found that the desired pH (from a solubility standpoint) of an aqueous formulation containing paclitaxel serum albumin is about pH 3.0 to about pH 4.8, that is, at about the pI or lower. With defatted HSA the desired pH is about 3 to 8.

The pH of the composition has also been found to affect the binding of to various serum albumins, including bovine, dog, horse, sheep and human, to different drugs and probes, including anthracyclines 4′-iodo-4′-deoxydoxorubicin (IDX) and 4-demethoxy-daunorubicin (DDN), warfarin and dansylsarcosine, and thiopental. Rivory et al. (1992) Biochem. Pharm. 44:2347-55; Panjehshahin et al. (1992) Biochem. Pharm. 44:873-9; Altmayer et al. (1990) Methods Find Exp. Clin. Pharm. 12:619-24; Wanwimolruk et al. (1982) Biochem. Pharmacol. 31:3737-43; Lassman et al. (1982) Naunyn-Schmiedeberg's Arch Pharmacol. 320:189-95.

The amount of paclitaxel administered to the patient will depend on several variables, such as the particular taxane used, the time course of administration, the condition of the individual, the desired objective, the extent of disease, how many doses will be administered, and whether any other substances are being administered in combination with paclitaxel. Generally, the amount used will be as recommended by the manufacturer and/or based on empirical studies. The amount of a single administration can be about 0.1 to about 1000 mg per kg body weight, or about 0.1 to about 1000 mg per day. The amount of a single dosage can be, for example, at least about 10, at least about 20, at least about 25, at least about 30, at least about 50, at least about 100, at least about 125, at least about 150, at least about 200, at least about 250, at least about 300, at least about 350, at least about 400, or at least about 500 mg/m² body surface area. The dosage can also be less than about 500, less than about 400, less than about 350, less than about 300, less than about 250, less than about 200, less than about 150, less than about 100, less than about 50, less than about 30, less than about 25, less than about 20, or less than about 10 mg/m². Preferably, the dosage is at least about 200 mg/m². Also, preferably the dosage is less than about 300 mg/m². Any of these doses can be further subdivided into separate administrations, and multiple dosages can be given to any individual patient. Therapeutically effective amounts of paclitaxel have been reported in the literature. McGuire et al. (1989); Brown et al. (1991) J. Clin. Oncol. 9:1261-1267; Keren-Rosenberg et al. (1997); and Stadler et al. (1997) Eur. J. Cancer 33 (Suppl. 1): S23-S26. The paclitaxel formulation of the present invention can be prepared in vials of, for example, about 5, 10, 15, 25, 50, 100, 150, 200, 250, or 500 mg each in the storage stable format for reconstitution and administration. Administration can be given in a duration of about 6 hours, 3 hours, 150 minutes or less, about 2 hours or less, about 1 hour, or about 15 minutes or less. Preferably, administration is by a bolus, not previously possible because the concentration of paclitaxel available in Cremophor EL® has not been adequate and the side effects of Cremophor EL® have been too severe.

The term “bolus” includes a single injection, or any administration volume small enough to be rapidly administered without prolongation of administration, e.g., as an i.v. drip.

“Non-cloudy” or “optically clear” solutions are those with a particular optical density or turbidity. As described below, the pharmaceutically acceptable formulations can be obtained by mixing solutions of paclitaxel and serum albumin or from reconstituting a dried, storage-stable composition. In the case of the reconstituted and non-reconstituted composition, optical clarity is defined as having a turbidity equal to or less than about 0.1 optical density (O.D.) as measured spectrophotometrically at a wavelength of 600 nm, blanked against clear formulation reaction mixture containing all components except the paclitaxel. The mixture should also be free of visible particulates within 8 hours of incubation at room temperature, before and after centrifugation at 16,000×g.

Paclitaxel administration can cause some side effects, including leukopenia, myalgia, arthralgia, alopecia, diarrhea, nausea, vomiting, mucositis and peripheral neuropathy, some or all of which are attributable to the Cremophor EL® vehicle. McGuire et al. (1989); Einzig et al. (1991) Cancer Invest. 9:133-136; and Runowicz et al. (1993) Cancer 71 (suppl.): 1591-1596. The reduction of side effects has been reported to have been achieved by premedication (with, for example, diphenhydramine, dexamethasone or cimetidine), and/or by modulating the time over which a certain drug amount is administered. Brown et al. (1991); Stadler et al. (1997); and Seidman et al. (1997) Oncology 11 (Suppl. 2):20-28. For example, the infusion of paclitaxel in a Cremophor EL® vehicle was prolonged to 6 hours and repeated every 21 days. Brown et al. (1991). In another study, varying paclitaxel dosages were given as a 24-hour infusion. McGuire et al. (1989). For additional examples of paclitaxel dosages and administration schedules, see U.S. Pat. No. 5,665,761 and EP 783885. Treatments for side effects of paclitaxel include administration of intravenous fluids, antihistamines, a vasopressor, aminophylline, and/or corticosteroids. Weiss et al. (1990) J. Clin. Oncol. 8:1263-68; and Runowicz et al. (1993) Cancer 71:1591-96.

By a “physiologically acceptable vehicle” is meant any physiologically-acceptable liquid in which the paclitaxel and serum albumin remain in an optically clear solution. By “in solution” is meant that a particular ingredient (e.g., paclitaxel or serum albumin) is not precipitated, crystalized, bound to the experimental vessel (e.g., test tube), or otherwise removed from solution in the vehicle as determined by optical clarity. Paclitaxel, which is bound to serum albumin in solution is itself still considered to be “in solution.” Thus, a physiologically acceptable vehicle can include non-toxic levels of alcohols and salts, 5% dextrose or other sugars, saline, and other pharmaceutically acceptable excipients, and any combination of any of these solvents. Such excipients are well known and described, for example, in Remington's Pharmaceutical Sciences, 18th edition, Mack Publishing (1990). One example of a physiologically acceptable vehicle is McIlvaine buffer. The formulation can comprise a physiologically acceptable vehicle immediately prior to administration. However, in the initial steps, in which the albumin and paclitaxel are combined, the formulation can comprise a non-physiologically acceptable solvent, provided that such a solvent is later removed, e.g., in the drying process, and provided that the formulation comprises a physiologically acceptable vehicle immediately prior to administration.

The formulation of the present invention can further comprise an iso-osmotic amount of a tonicity agent. The term “tonicity agent” as used herein means an agent, which allows the pharmaceutical compositions of the present invention to have an osmotic pressure compatible with human serum. Typically suitable tonicity agents, which can be present in the preferred pharmaceutical compositions of the present invention, include sorbitol, mannitol, sodium chloride, glycine and dextrose. The preferred tonicity agent (when one is used), is sorbitol or mannitol but any pharmaceutically acceptable tonicity agent would also be acceptable.

The term “iso-osmotic” as used herein in reference to the amount of tonicity agent means the amount of the tonicity agent appropriate to make the pharmaceutical compositions of the present invention upon administration to a mammal iso-osmotic with the plasma of such a mammal. The iso-osmotic amount of tonicity agent varies with the tonicity agent used and may conveniently be measured in accordance with the procedures described in Remington's Pharmaceutical Sciences, Gennaro, ed., 1990, 18th Edition, Mack Publishing Co., Easton, Pa., Chapter 79 entitled “Tonicity, Osmoticity, Osmolality and Osmolarity”, pages 1481-1498 at 1488-1491. The iso-osmotic amount of mannitol, the preferred tonicity agent, is preferably about 35 to 45% by weight basis total weight of all ingredients in the composition.

The paclitaxel formulations of the present invention should be essentially free of toxic ingredients such as Cremophor EL®. By “essentially free” is meant that the paclitaxel formulation contains less than about 1% (w/v or v/v) of Cremophor EL®, more preferably less than about 0.1% Cremophor EL®, more preferably less than about 0.01%. Cremophor EL®, if present as a solvent for paclitaxel, can be removed in the process of preparing the paclitaxel formulation of the present invention, e.g., in the lyophilization step. Most preferably, Cremophor EL® is not an ingredient in the paclitaxel formulations of the present invention and is not present in them at detectable levels.

The present invention also provides storage-stable formulations (compositions) containing paclitaxel, a serum albumin and, optionally, in combination with one or more pharmaceutically acceptable vehicles, excipients, diluents or adjuvants. The composition can be in the form of a concentrated aqueous composition or a dried composition from which the solvent (e.g., water) has been removed. The dried or concentrated formulation can be reconstituted to obtain pharmaceutically acceptable formulations. The drying process can be by any method known in the art. Preferably, the drying process is lyophilization. Methods of drying are known in the art and disclosed, for example, in Remington: The Science and Practice of Pharmacy, Vol. II, and Pharmaceutical Dosage Forms: Parenteral Medications, Vol. 2, nineteenth edition, Avis et al. In one embodiment, the composition prior to drying comprises McIlvaine buffer or certain saline solutions, including dilutions of saline. The reconstituted composition can be made at least 2-10 times more concentrated than the original composition. The invention thus encompasses reconstituted compositions.

The reconstituted compositions can have the same or a different concentration of paclitaxel than the composition prior to drying. The more concentrated, the smaller the volume.

Effect of the concentration of paclitaxel in the reconstituted formulation on the dose volume for injection:

| Paclitaxel conc. | | Paclitaxel | | Dose |
|---|---|---|---|---|
| (μg/ml) | Molar ratio | (mg) | HSA (g) | volume (ml) |
| 50 | 1:1 | 30 | 2.34 | 600 |
| 100 | 1:1 | 30 | 2.34 | 300 |
| 200 | 1:1 | 30 | 2.34 | 150 |
| 400 | 1:1 | 30 | 2.34 | 75.0 |
| 600 | 1:1 | 30 | 2.34 | 50.0 |
| 800 | 1:1 | 30 | 2.34 | 37.5 |
| 1000 | 1:1 | 30 | 2.34 | 30.0 |

In one embodiment, after reconstitution, the present formulation comprises an optically clear pharmaceutically acceptable formulation of paclitaxel and an isolated, natural or recombinant albumin, or an amino-acid-modified derivative thereof, essentially free of surfactants, organic solvents, and oils, and derivatives thereof, wherein the paclitaxel concentration is between about 0.05 to 2 mg/ml, preferably 0.2 to 1.0 mg/ml. An “oil” as used herein is any of various viscous, water-immiscible liquids that are soluble in organic solvents such as ether or naphtha; oils include, but are not limited to, Cremophor EL®.

Paclitaxel can be administered as the sole active agent, or in conjunction with one or more additional active substance and/or therapeutics, depending on the context of administration (i.e., desired end result, condition of the individual, and indications). “In conjunction with” means that the paclitaxel formulation is administed prior to, concurrently, or after the other active substance or therapy. These agents can have an independent activity, an activity related to that of paclitaxel, or can specifically enhance the activity of paclitaxel. In the last category, EP 781552 and EP 787716 describe compounds that enhance paclitaxel activity. Other substances that can be administered in conjunction with a paclitaxel include, but are not limited to, cytokines, and other substances believed to be effective in treating and/or preventing cancer. Such additional agents include, but are not limited to, G-CSF, GM-CSF, IL-4), IGF-I, analide derivatives, antiarthritics, antibodies specific to cancer cells, antineoplastics (e.g., carboplatin, cyclophosphamide, estramustine phosphate, and etoposide), doxombicin, immunosuppressants (e.g., cisplatin and cyclophosphamide), steroidal and non-steroidal hormones (e.g., cortisone), transduction inhibitors, and vitamins (e.g., vitamin C). Bolis (1995) Semin. Oncol. 22 (suppl. 14):32-34; Bolis et al. (1997) Semin Oncol. 24 (suppl. 2):S2-23-S2-25; Fleming et al. (1996) Cancer 77:2308-2312; Weiss et al. (1990); Runowicz et al. (1993) Cancer 71:1591-96; WO 94/10995; and U.S. Pat. Nos. 5,496,804, 5,716,612, and 5,728,687. In addition, paclitaxel can be administered in conjunction with agents known to reduce the side effects of paclitaxel. Such agents include, but are not limited to, G-CSF, GM-CSF, corticosteroids (suck as dexamethasone), diphenhydramine, and antihistamines (such as H₁ and H₂ receptor antagonists, including cimetidine, famotidine, and ranitidine).

Paclitaxel Formulations

The present invention provides pharmaceutically acceptable formulations containing paclitaxel, a serum albumin and a pharmaceutically acceptable vehicle. The serum albumin is preferably non-aggregated; or loosely aggregated; and predominantly monomeric. Preferably, the serum albumin is at least about 50%, at least about 60%, at least about 70%, at least about 80%, or at least about 90% monomeric monomeric. Preferably, the albumin is defatted as this has now been found to improve the binding of paclitaxel across a wide range of ionic strengths and pH. This is not the case with fatted albumin, which has optimal binding only at acidic pH. In the case of fatted albumin, increasing ionic strength of the composition increases binding of the paclitaxel, thus, increasing the concentration of these potentially unacceptable ions. The formulation can comprise any molar ratio of paclitaxel to serum albumin which allows the albumin and paclitaxel to remain in solution, and if preferably about 1:4 to about 2:1 (paclitaxel:albumin). It is anticipated that ratios of 3:1 and possibly even 4:1 can be achieved according to the invention described herein, by controlling the rate of addition of the paclitaxel to the albumin solution to a degree that does not interfere with continued stability during processing. The paclitaxel is bound to serum albumin non-covalently (e.g., via hydrogen-bonding, hydrophobic interactions and/or electrostatic interactions).

Unexpectedly, not all amounts of paclitaxel, solvent and albumin, and ratios between them, have been found to result in optically clear formulations enduring for a period of 8 to 24 hrs. Preferably, the paclitaxel is at a concentration and/or ratio of paclitaxel:organic solvent and/or ratio of paclitaxel:albumin such that the paclitaxel remains in solution. In the pharmaceutically acceptable compositions, the paclitaxel can be in a concentration greater than about 50, greater than about 100, greater than about 200, greater than about 300, greater than about 400, or greater than about 500, or greater than about 600 μg/ml. The paclitaxel in the pharmaceutically acceptable composition can also be at a concentration of less than about 600, less than about 500, less than about 400, less than about 300, less than about 200, less than about 100, or less than about 50 μg/ml. Preferably, the paclitaxel is present at about 50 to about 500 μg/ml. Preferably, the solvent is an alcohol, more preferably ethanol. Preferably, the organic solvent is present at a concentration at which the paclitaxel remains in solution for at least 8 hours and preferably 24 hours at room temperature. For instance, a concentration of about 2% to about 15% ethanol is effective for a paclitaxel formulation of about 50 to about 600 μg/1 ml. We have surprisingly found that the preferred ethanol concentration is about 2% to 10% and most preferably, 4% to 6%.

Preferably, the paclitaxel is bound to albumin in a ratio such that they remain in solution. Preferably, the paclitaxel is in a ratio with serum albumin of greater than about 1:5, greater than about 1:4, greater than about 1:2, greater than about 1:1, or greater than about 2:1 (paclitaxel:albumin). More preferably, the paclitaxel is present at a molar ratio of between about 1:4 to about 1:0.5 (paclitaxel:albumin) in about 0.2 mg/ml paclitaxel and about 4% ethanol.

In a preferred method of preparing the paclitaxel formulation, a solution comprising paclitaxel in a vehicle is combined slowly (e.g. dropwise) with a separately-prepared solution containing albumin in a vehicle. The paclitaxel solution can, as an non-limiting example, be added to the serum albumin solution dropwise at a controlled rate; this rate can be, in a non-limiting example, between 0.1 ml/min and 10 ml/min, e.g., 1 ml/min or slower, and the drop size can be 8 to 20 μl. During the addition process, the solutions can be mixed, e.g., at a speed sufficient to produce a vortex. Preferably, the vehicle comprises an organic solvent, such as an alcohol, preferably ethanol. Preferably, the solvent concentration allows paclitaxel and albumin to remain in solution, such as a concentration of about 2% to about 25% ethanol in a solution of 50 up to about 600 g/ml paclitaxel, or a concentration of about 2% to about 25% ethanol in a solution of up to about 230 mg/ml human serum albumin. Paclitaxel and albumin can be present, for example, in a ratio of about 1:0.5 to about 1:10 (paclitaxel:albumin) in about 2% to about 10% ethanol. In addition, the serum albumin can be defatted or non-defatted, the state being appropriate to maximize solubility of paclitaxel, such as defatted serum albumin in a 2:1 molar ratio with about 50 to about 600 μg/ml paclitaxel in 5% ethanol. Preferably, the pH of the solution is such that paclitaxel and serum albumin remain in solution, such as a pH of about 4.8 or lower for a solution of about 50 to about 500 μg/ml paclitaxel at an approximately 1:4 molar ratio with serum albumin in about 5% ethanol; or a pH of about 3.2 to about 4.0 for a solution of up to about 600 μg/ml paclitaxel at an approximately 1:4 molar ratio with serum albumin in about 5% ethanol.

However, based on the present disclosure, additional amounts and ratios of ingredients which result in non-cloudy formulations can be readily determined by simply mixing or carefully pumping the ingredients in various amounts and ratios and slow rates of pumping or addition of paclitaxel, and checking for cloudiness. Preferably, the paclitaxel is added slowly, while the solution is being mixed. The cloudiness of preparations containing particular concentrations of paclitaxel, organic solvent and serum albumin, and ratios between these ingredients, can be measured qualitatively (visual inspection for clouding, precipitation or crystal-formation) or quantitatively (spectrophotometric measurement of OD₆₀₀), ELISA LSC (liquid scintillation counter), etc. Preferably, the step of combining the paclitaxel solution and albumin solution is performed slowly (e.g., as described above), and the solution re-checked for clouding or precipitation. The preferred solutions of serum albumin, paclitaxel, and aqueous and organic solvents are optically clear.

Commercial Feasibility

The formulations as described herein represent the first commercially feasible method for using a serum albumin to administer paclitaxel. Albumin is an expensive ingredient in order to produce a commercially available, pharmaceutically acceptable albumin-bound paclitaxel, the drug must be bound reversibly to the albumin in a high molar ratio. The commercial absence of any such paclitaxel formulations indicates that this goal has not yet been attained.

The economic feasibility of the present formulations is based on the following:

| | Total price for |
|---|---|
| | a 30-mg dose |
| The present BMS (Bristol-Myers Squibb) | $170 |
| paclitaxel formulation | |
| Target for NBI (Novopharm Biotech Inc.) | $140 |
| paclitaxel formulation | |
| Breakdown of the NBI paclitaxel formulation | |
| total costs | Price for 30-mg dose |
| 1. The target for formulation ingredients is 10% | $14 |
| 2. The target for Packaging, Marketing | $126 |
| and Profits is 90% | |
| Breakdown of the NBI paclitaxel | |
| formulation (main ingredients) | |
| Main ingredients costs of $14 for a 30-mg dose | Cost for 30-mg dose |
| 1. Paclitaxel | $7 |
| 2. Human serum albumin | $7 |

Based on binding molar ratio:

Estimation of main ingredients cost at different binding molar ratios for a 30-mg dose of paclitaxel

| | | | | | Ingredients |
|---|---|---|---|---|---|
| Molar | Paclitaxel | HSA | Paclitaxel | HSA | Total |
| ratio | (mg) | (g) | Cost | Cost⁽¹⁾ | Cost |
| 1:10 | 30 | 23.4 | $7 | $74.90 | $81.90 |
| 1:5 | 30 | 11.7 | $7 | $37.40 | $44.40 |
| 1:2 | 30 | 4.7 | $7 | $15.00 | $22.00 |
| 1:1 | 30 | 2.34 | $7 | $7.49 | $14.50 |
| 1:0.5 | 30 | 1.17 | $7 | $3.74 | $10.70 |

⁽¹⁾The fair 1999 market value of HSA is approximately $3.20 per gram.

In one embodiment, the composition comprising paclitaxel, a serum albumin and a physiologically acceptable vehicle is dried, stored as a dried storage-stable composition, and then resolubilized prior to administration. In a preferred embodiment, the drying process is lyophilization. In one embodiment, the composition prior to drying comprises McIlvaine buffer. In another embodiment, the composition is reconstituted after lyophilization with a physiologically acceptable vehicle, such as McIlvaine buffer, a sugar solution such as dextrose or glucose, water, or certain saline solutions, so as to attain a pharmaceutically acceptable composition.

In another embodiment, the composition comprising paclitaxel, a serum albumin and a physiologically acceptable vehicle can be coated onto an implantable device such as a stent or wrap. In some embodiments, the device is catheter-based and/or used in conjunction with surgery. In some embodiments, the coating can prevent restenosis, local tumor growth or tissue over-growth and/or chronic inflammation.

The paclitaxel formulation can further comprise an additional ingredient such as a detergent, a glycol, or derivative thereof (such as polyethylene glycol). Antioxidants (such as DTE, DTT, sodium metabisulfite, thioethanolamine thioacetic acid required to maintain HSA in monomer form) and polyols (such as mannitol, sorbitol, etc.) for cryoprotection or other stability considerations are indicated formulation ingredients. Use of such antioxidants to limit aggregation of serum albumin is known in the art. These additional ingredients should be non-toxic and/or at a low concentration (e.g., less than about 5%, less than about 2%, or less than about 1%).

The paclitaxel formulation can also comprise an additional therapeutic agent. Such additional agents include, but are not limited to, G-CSF, GM-CSF, IL-4, IGF-I, analide derivatives, antiarthritics, antibodies specific to cancer cells, antineoplastics (e.g., carboplatin, cyclophosphamide, estramustine phosphate, and etoposide), doxombicin, immunosuppressants (e.g., cisplatin and cyclophosphamide), steroidal and non-steroidal hormones (e.g., cortisone), transduction inhibitors, and vitamins (e.g., vitamin C).

Preferably, none of these methods for preparing paclitaxel involve the use of Cremophor EL® or any other toxic solvent.

The paclitaxel formulations of the present invention prepared in the manner described herein, containing paclitaxel, serum albumin and an aqueous solvent (except in the case of the dried, storage-stable composition), can be used to treat any number of diseases. These diseases include cancer, primarily ovarian and breast cancer, but also cancer affecting cells of the bladder, blood, bone, brain, cervix, colon, epithelium, digestive tract, head/neck, kidneys, liver, lung, mouth, pancreas, prostate gland, skin, stomach, testicles, or tongue. In addition, paclitaxel formulations of the present invention can be used to treat Alzheimer's disease, kidney disease, peripheral neuropathy, psoriasis, restenosis, rheumatoid arthritis, systemic lupus erythematosus, surgical adhesions, or tissue overgrowth after surgery.

Paclitaxel Administration

Pre-Treatment

Prior to administration of the formulations of the present invention, the patient can be pre-treated with any agent known to reduce the side effects of paclitaxel. Such pre-treatment agents include, but are not limited to, G-CSF (granulocyte colony-stimulating factor), GM-CSF (granulocyte macrophage colony-stimulating factor), corticosteroids (such as dexamethasone), diphenhydramine, and antihistamines (such as H₁ and H₂ receptor antagonists, including cimetidine, famotidine, and ranitidine). Preferably, the pre-treatment agent is G-CSF or GM-CSF. Weiss et al. (1990); Runowicz et al. (1993) Cancer 71:1591-96; and U.S. Pat. Nos. 5,496,804, and 5,728,687.

The pre-treatment agent is administered, for example, less than about 30 minutes, less than about an hour, less than about 3 hours, less than about 6 hours, less than about 12 hours, less than about 24 hours, less than about 48 hours, or less than about 96 hours, prior to paclitaxel administration. The pre-treatment agent can be administered more than once prior to, during, or after paclitaxel administration. The amount and timing of the pre-treatment agent will vary with the agent. For example, GM-CSF can be administered as a single daily subcutaneous dosage of 250 μg/m²; Dexamethasone can be administered at a dosage of about 20 mg orally, about 14 to about 12 hours and about 7 to about 6 hours prior to paclitaxel, or at a dosage of 8 mg about 24, 18, 12, and 6 hours prior to paclitaxel administration; an H₂ receptor antagonist (e.g., ranitidine, 50 mg, or famotidine, 20 mg) can be administered 30 minutes prior to paclitaxel administration; and/or Cimetidine can be administered at a dosage of about 300 mg intravenously (IV) and Diphenhydramine at about 25 to about 50 mg orally or IV, about 30 minutes prior to paclitaxel. If the pre-treatment agent is G-CSF, the amount can be about 5 mg/kg/day to about 20 mg/kg/day. If the pre-treatment is GM-CSF, it can be given at 0.05 μg to 500 μg/kg body weight. Flaming et al. (1996) Cancer 77:2308-2312; Bolis (1995) Sem. Oncol. 22 (suppl. 14): 32-34; Bolis et al. (1997) Sem. Oncol. 24:S2-23-S2-25; Weiss et al. (1990) J. Clin. Oncol. 8:1263-1268; and U.S. Pat. Nos. 5,162,111, 5,496,804, 5,616,608, 5,665,761, and 5,731,334. The pre-treatment agent can also be administered throughout paclitaxel administration and/or after paclitaxel administration. For example, if paclitaxel is administered once weekly, the pre-treatment agent can be administered prior to the first administration of paclitaxel, daily or twice-daily or weekly, and/or subsequent to the final paclitaxel administration.

Dosage Amounts and Duration

The amount and duration of administration of the present paclitaxel formulations will vary according to the indication and the condition of the patient.

A single paclitaxel dosage can be at least about 15, at least about 25, at least about 50, at least about 100, at least about 150, at least about 200, at least about 250, at least about 300, at least about 400, or at least about 500 mg/m². The paclitaxel dosage can be less than about 500, less than about 400, less than about 300, less than about 250, less than about 200, less than about 150, less than about 100, less than about 50, less than about 25, or less than about 15 mg/m². Preferably, the paclitaxel dosage is at least about 200 mg/m² and less than about 600 mg/m². McGuire et al. (1989); Brown et al. (1991); Wiemik et al (1987) Cancer Research 47:2486-2493; and Kris et al. (1986) Cancer Treat. Rep. 70, No. 5.

As now provided herein, a dosage of paclitaxel can be administered in a single administration (bolus). The pharmaceutically acceptable composition can also be administered as several administrations, and/or as a prolonged dosage (drip). Multiple dosages of paclitaxel can be administered, e.g. at three-week intervals. For example, the paclitaxel can be administered as a drip over a 6 hour duration, which is to be repeated every 21 days; as an infusion with a duration of less than about 24 hours, less than about 18 hours, less than about 12 hours, less than about 6 hours, less than about 150 minutes, less than about 60 minutes, less than about 30 minutes, or less than about 15 minutes; at a dosage of between about 200 mg/m² to about 600 mg/m² during a single duration of less than about 150 minutes, less than about 60 minutes, or less than about 15 minutes; at a dosage of between about 135 mg/m² and about 175 mg/m² or between about 150 and about 225 mg/m² in a single 3-hour infusion; about 200 to about 600 mg/m² in a single 6-hour infusion; at about 250 mg/m² over a 24 hour infusion repeated every 21 days; or in escalating step dosages of about 15 to about 230 mg/m², given as 150-minute IV infusions every 21 days. Periodic administrations (e.g., about every one, two or three weeks) can be given for about six to about eighteen months, preferably at least about six months, most preferably about twelve months. U.S. Pat. No. 5,665,761; McGuire et al. (1989); Kris et al. (1986); Keren-Rosenberg et al. (1997); and Stadler (1997); and Brown et al. (1991). Preferably, the paclitaxel formulation is administered during a duration of about 150 minutes or less, about 15 minutes or less, or as a single bolus.

Drying and Resolubilization

The composition comprising paclitaxel, a serum albumin and a physiologically acceptable vehicle can be dried, stored as a dried composition, and then resolubilized prior to administration.

Coating the Composition onto an Implantable Medical Device

In another embodiment, the composition comprising paclitaxel, a serum albumin and a physiologically acceptable vehicle can be coated onto an implantable device such as a stent or wrap. In some embodiments, the device is catheter-based (e.g., a stent, a balloon or drug-delivery catheter) and/or used in conjunction with surgery. In some embodiments, the coating prevents restenosis, local tumor growth or tissue over-growth and/or chronic inflammation. In some embodiments, these coated devices can be used in treating indications such as cardiovascular disease, psoriasis, rheumatoid arthritis, multiple sclerosis, or a cancer such as a gastrointestinal cancer, such as esophageal cancer.

Stents are often inserted into body ducts such as blood lumens to prevent collapse thereof. However, restenosis (recurrence of blockage) can often occur. Restenosis is often a complication of vascular graft insertion for kidney hemodialysis patients and surgical bypass procedures. Paclitaxel can interfere with the processes leading to restenosis, and coating the stent prior to implant should therefore limit restenosis.

Stents can also be inserted into tracheobronchial tubes, genito-urinary ducts, biliary ducts, or the esophagus or other gastrointestinal tract spaces, or other lumens. These lumens may become occluded by overgrowth of adjacent tumors. An esophageal stent, for example, can be inserted in a patient whose esophagus has become obstructed by tumor tissue to such an extent that eating is difficult or impossible. Although this procedure does not prolong life, it can improve the quality of life for the patient and shorten the time spent in hospital. Coating gastrointestinal stents with a composition of the present invention could reduce or prevent tumor overgrowth of the stent and increase the clinical effectiveness of the device. In many cases of cancerous overgrowth, the coating should have direct cytotoxic effect on the tumor cells themselves. Alternatively, a composition of the present invention can be coated onto a wrap. While a stent is implanted inside a body cavity such as a lumen, a wrap is applied outside, e.g. wrapped as a thin film around a damaged blood vessel.

The following examples are provided to illustrate but not limit the invention.

### EXAMPLE 1

### Pharmaceutical Formulations Comprising Paclitaxel, Serum Albumin and an Aqueous Solvent

Briefly, in one method of preparing pharmaceutical formulations comprising paclitaxel, serum albumin and a physiologically acceptable vehicle, for example, separate solutions of paclitaxel and serum albumin in the vehicle are first prepared. The vehicle can comprise an organic solvent and the same or different vehicles can be used for the paclitaxel and albumin solutions. The optimal concentrations of paclitaxel and organic solvent, ad ratios between these two ingredients, are determined. The optimal concentrations of serum albumin and organic solvent, and the ratios between these two, are separately determined. The paclitaxel solution is then combined, slowly, with the albumin solution, at an acidic pH, as discussed above. The solutions comprising albumin, paclitaxel and both ingredients should be checked for clouding, precipitation, crystal-formation, and the like. Optically clear solutions are preferred.

1. A. Summary of Optimal Concentrations of Ingredients

Practically, to enable the binding of paclitaxel (when solubilized in an organic solvent such as an alcohol such as ethanol) to human serum albumin, (i) the concentration of the organic solvent at any time must not exceed the concentration that would cause the denaturation or the precipitation of the albumin, and (ii) the paclitaxel concentration must not be too high at any time such that it would precipitate out before interacting with the albumin. The recommended strategy for developing a concentrated and optically clear formulation of Ptx bound to HSA at a high molar ratio would be as follows:

a) Establish an initial concentration of organic solvent and Ptx for the binding reaction.

The maximum working organic solvent concentration is established with HSA, since it is precipitated and denatured by high concentration of a solvent such as ethanol. This was carried out by mixing a fixed concentration of HSA with ethanol in the concentration range of 5% to 40% (v/v) at room temperature and measuring the solution turbidity as a function of time. Human serum albumin was found stable (clear solution) in aqueous ethanolic solutions at concentrations of up to 25% (v/v), when ethanol was added dropwise to the serum albumin solution. For subsequent studies, we established the practical maximum working ethanol concentration to be 20% instead of 25%.

Unlike HSA, the solubility of Ptx decreases with decreasing ethanol concentration. At a fixed ethanol concentration, the rate of visible crystal formation decreases with the Ptx concentration. Therefore the starting Ptx concentration was established as the concentration at which the visible precipitation of Ptx in dilute aqueous ethanolic solution is not instantaneous. More specifically, the solubility of paclitaxel in aqueous ethanol solutions (5% to 20% v/v) was analyzed in the concentration range of 25 to 500 μg/1 ml paclitaxel. In initial studies, solutions of 500 μg/ml paclitaxel were found to be cloudy in solutions of up to 25% ethanol. Apparently clear paclitaxel solutions were obtained at less than 100 μg/ml of paclitaxel, after a 1-hr incubation at room temperature. During prolonged incubation (12 h), all paclitaxel solutions formed precipitates, the extent of which depended on the concentration of both ethanol and paclitaxel. For future studies, 50 μg/ml paclitaxel was selected as being stable in 15% to 20% ethanol, at neutral pH for at least 1 hour.

b) Establish the initial unoptimized molar binding ratio of Ptx to a commercial HSA in a physiological saline solution at neutral pH.

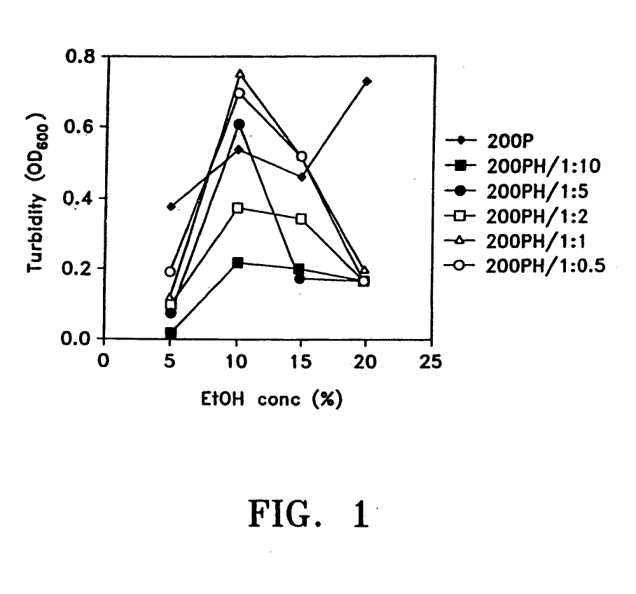

Molar ratio studies were carried out with increasing concentration of Ptx starting at 50 μg/mL at a fixed concentration of HSA at neutral pH, and the solubilizing effect of HSA evaluated by measuring the solution turbidity (FIG. 1). Low turbidity was observed at a Ptx to HSA molar ratio of 1:10 and 1:5. Higher molar ratios were instantaneously cloudy. The most stable solution was the 1:10 molar ratio. Thus establishing the unoptimized and uninventive binding molar ratio between 1:10 and 1:5.

c) Optimize the binding parameters to achieve inventive higher molar ratio and concentrated stable Ptx/HSA formulation.

To achieve higher molar ratio of concentrated Ptx/HSA formulation, the conditions that optimize the interaction of Ptx to HSA and stability of the complex as listed in section 1B were evaluated.

We have established that Ptx can be bound to HSA at a high molar ratio with high recovery of soluble Ptx when the commercial HSA solution is acidified to pH 3.2-3.8, and optimally diluted Ptx in absolute ethanol added slowly to the HSA in 0.2-0.85% NaCl or McIlvaine buffer solution with constant mixing to a final Ptx concentration of up to 600 μg/mL and ethanol concentration not exceeding 10% (v/v), preferably 4%, and to molar ratio of up to 2:1 Ptx to HSA, with a demonstrated stability of at least 8-24 h. Other excipients in the formulation are sorbitol added at a concentration of 4% (w/v) and antioxidant such as dithiotreitol and cysteine at 0.7 mM each. Defatted HSA equally bound Ptx at high molar ratio in the acidic pH range. The observed solubilizing effect of Ptx was dependent on human serum albumin, since the control solutions (lacking this protein) turned turbid under the experimental conditions. These and additional results are explained in greater detail below.

Molar ratios, pH and ethanol studies provided surprising results. On the basis of turbidity, ELISA and radioactive assays to assess the solubilization of paclitaxel in the reaction mixtures, lower ethanol concentrations (2-8%) and acidic pH range (3.2-3.8) unexpectedly resulted in increased solubility of paclitaxel than did higher ethanol concentrations (10-20%) and higher pH (FIGS. 1 and 9-11). For example, a ratio of about 1:0.5 to about 1:10 (paclitaxel:albumin) with a fixed concentration of 200 μg/ml paclitaxel and 5% ethanol allowed solubility of both paclitaxel and albumin. This effect was dependent on human serum albumin, since the control solutions (lacking this protein) turned turbid under the experimental conditions. Quantitation of paclitaxel binding was carried out by estimating unbound paclitaxel by ELISA and HPLC for non-radioactive formulations and LSC for formulations spiked with radioactive Ptx. A very high degree of binding was obtained, when paclitaxel was added to human serum albumin at molar ratios of 1:1 and 1:2, with paclitaxel fixed at concentrations of either 50 or 100 mg/ml. These and additional results are explained in greater detail below.

1. B. Evaluation of the Optimal Conditions for Binding Paclitaxel to Human Serum Albumin

Different experimental conditions were evaluated to analyze the binding of paclitaxel to HSA. In these experiments the following issues were examined:
The ethanol concentration.
The reaction pH.
The order and rate of mixing Ptx and HSA.
The type of HSA, such as defatted and undefatted HSA.
The formulation stabilizers, such as antioxidants, polyols, and filling under inert gas.
The buffer systems and ionic strength.
The reaction temperature.
   1.C. Effect of Ethanol Concentration on the Solubility of Paclitaxel

The solubility of paclitaxel in aqueous solutions was found to depend on a number of factors, including the concentration of organic solvent, the concentration of paclitaxel, and the temperature. These experiments had two objectives: (i) determine the effect of ethanol concentration on the solubility of a fixed amount of paclitaxel in a physiological saline solution and (ii) determine the effect of paclitaxel concentration in aqueous ethanol solution on the solubility of paclitaxel at room temperature.

Experimental Procedure

The concentrations of ethanol tested ranged from 5 to 25% (v/v). The paclitaxel concentration was kept constant at 0.5 mg/ml.

Reagents Preparation
1. A 1 ml stock solution of Paclitaxel (10 mg/ml in ethanol) was prepared in a small vial and designated the 10 Ptx stock solution.
2. A 20-ml physiological saline stock solution was also prepared in a small bottle (or flask) and designated the 1× saline stock solution.

Procedure
1. 50 μL of 10 Ptx stock solution was aliquoted into 5 small conical test tubes and preincubated at room temperature.
2. Ethanol was added to each set of 5 tubes to give final concentrations of 5, 10, 15, 20 and 25% (v/v) in physiological saline according to the Table 1.
3. The volume in each tube was brought up to 1 ml with physiological saline solution to give a final paclitaxel concentration of 0.5 mg/ml.

4. The tubes were incubated at room temperature and observed at 0 h, 1 h and 3 h for precipitate formation. The results were recorded as + or − turbidity formation.

TABLE 1
The experimental design for the analysis of paclitaxel
solubility in aqueous ethanol solution.

| | | Amount of | | |
|---|---|---|---|---|
| Sample | Ethanol conc. | Paclitaxel⁽²⁾ | Amount of | Amount of saline |
| Name⁽¹⁾ | (%) | (μL) | ethanol (μL) | (μL) |
| Pt-5.c | 5 | 50 | 0 | 950 |
| Pt-10.c | 10 | 50 | 50 | 900 |
| Pt-15.c | 15 | 50 | 100 | 850 |
| Pt-20.c | 20 | 50 | 150 | 800 |
| Pt-25.c | 25 | 50 | 200 | 750 |

⁽¹⁾Samples were labeled as Pt-5 to Pt-25, where P is for paclitaxel; and t is 4, 23, 37 or 45 for different incubation temperatures in ° C.; and c is the fixed paclitaxel concentration in μg/ml. For instance, a test condition of paclitaxel (0.5 mg) in 5% aqueous ethanol solution (1 ml) incubated at 23° C., was labeled as T23-5.500.
⁽²⁾10 Ptx stock solution: 10 mg/ml paclitaxel in absolute ethanol.

Results

The solubility of paclitaxel in aqueous ethanol solutions (5% to 25%) was analyzed in the concentration range of 25 to 500 μg/ml. The solubility of paclitaxel at a fixed concentration of 500 μg/ml was analyzed in 5, 10, 15, 20 and 25% ethanol. Paclitaxel solutions in 20% ethanol or less turned cloudy within 5 minutes of incubation at room temperature. After 1 hr. all solutions became cloudy, suggesting that paclitaxel at 500 μg/ml was not soluble in saline solution containing up to 25% ethanol. Clear paclitaxel solutions were obtained at less than 100 μg/ml of paclitaxel, after a 1-h incubation at room temperature. During prolonged incubation (24 hr), all solutions of paclitaxel and ethanol formed precipitates, the extent of which depended on the concentration of paclitaxel and ethanol. At concentrations of 50 μg/ml or lower, paclitaxel solutions remained clear for at least 3 hours. Thus, the 50 μg/ml concentration was selected as the starting concentration for further studies.

1.D. Effect of Ethanol Concentration on the Stability of Human Serum Albumin

At high concentrations, ethanol causes the denaturation of most proteins. Without wishing to be bound by any particular theory, inventors thought that ethanol can reduce water availability to below the level at which proteins remain functionally and structurally stable. Since paclitaxel stock solutions are prepared in 100% alcohol, the analysis of the effect of ethanol concentration on the stability (precipitation) of HSA was required. This experiment had one objective: to determine the maximum working ethanol concentration that had minimal effect on the stability of HSA in aqueous ethanol solutions.

Experimental Procedure

The effect of ethanol concentration on HSA solubility was analyzed at different amounts of ethanol ranging from 5 to 25% (v/v) in aqueous reaction mixtures at a fixed concentration of HSA (100 mg/ml).

Reagent Preparation
1. A 10 ml stock solution of HSA (200 mg/ml aqueous solution) was prepared in a small vial and designated the 200 HSA stock solution.
2. Three 20-ml saline stock solutions of 1×, 2× and 4× the normal NaCl concentration in physiological saline were also prepared in small bottles (or flasks) and designated the 1× Saline, 2× Saline and 4× Saline stock solutions, respectively.

Procedure

1. Three sets of conical tubes were labeled as 1×, 2× and 4× corresponding to the 3 different saline stock solutions, respectively.
2. 500 μL of 200 HSA stock was aliquoted into all 3 sets of tubes, and was preincubated at room temperature.
3. Different amounts of appropriate saline stock solution were added to each set of tubes according to the Table 2.
4. Ethanol was added to each set of 5 tubes to give final concentrations of 5, 10, 15, 20 and 25%, and none to the control tube, according to the Table 2.
5. The tubes were incubated at room temperatures and observed at 0 h, 1 h and 3 h for precipitate formation. The results were recorded by + or − turbidity formation.

Sample Analysis

TABLE 2
The experimental design for the analysis of HSA
solubility in aqueous ethanol solution.

| Sample | Ethanol conc. | Amount of | Amount of | Amount of |
|---|---|---|---|---|
| name⁽¹⁾ | (%) | HSA⁽²⁾ (μL) | ethanol (μL) | saline (μL) |
| Ht-0 | 0 | 500 | 0 | 500 |
| Ht-5 | 5 | 500 | 50 | 450 |
| Ht-10 | 10 | 500 | 100 | 400 |
| Ht-15 | 15 | 500 | 150 | 350 |
| Ht-20 | 20 | 500 | 200 | 300 |
| Ht-25 | 25 | 500 | 250 | 250 |

⁽¹⁾Samples were labeled as Ht-0 to Ht-25; where H is for HSA; and t is 4, 22 or 37 for different incubation temperatures in ° C.
⁽²⁾The 200HSA aqueous stock solution concentration is 200 mg/ml (the commercial 20% solution).

Results

The stability of HSA at 100 mg/ml was analyzed in a saline solution containing 5, 10, 15, 20, 25, 35, or 40% (v/v) ethanol. HSA solutions containing 30% ethanol showed some increase in turbidity, and those with 35% ethanol or greater turned cloudy instantly at room temperature. However, clear HSA solutions were obtained in aqueous ethanol solutions at concentrations of up to 25% (v/v). The method of addition was very critical. Ethanol can be successfully added to an aqueous HSA solution dropwise (8 to 20 μl/drop) with constant mixing. Other methods, including addition of the ethanol all at once, resulted in some precipitation and/or denaturation of HSA even at lower ethanol concentration. Addition of HSA to ethanol followed by adjustment of the reaction mixture volume with vehicle resulted in some precipitation and/or denaturation of HSA, the extent of which increased with the amount of ethanol used, and the incubation time. For subsequent studies, the maximum working ethanol concentration was established at 20%, in aqueous vehicle. Under these conditions, there was assurance that the formation of precipitation (cloudy solutions) in the reaction mixtures containing both paclitaxel and HSA, at neutral pH, was not due to precipitation or denaturation of HSA, but rather to the insolubility of paclitaxel.

1.E. Effect of Different Ethanol Concentrations and Molar Ratios of Paclitaxel and Human Serum Albumin on Paclitaxel Binding

Efficient binding of paclitaxel to HSA is influenced by the solubility of paclitaxel, the optimal concentrations of paclitaxel and HSA, and the ethanol concentration. Other factors that may influence this binding are the reaction temperature and time, the pH and the ionic strength of the solutions, the nature of HSA preparations, and ratios of paclitaxel and HSA. This experiment had two objectives: (i) determine the effect of ethanol concentration on the binding of paclitaxel to HSA, and (ii) determine the effect of molar ratio of paclitaxel and HSA added to the reaction mixtures on the binding efficiency at room temperature.

Experimental Procedure

The experimental procedure was as described below. An important experimental consideration was that paclitaxel in ethanol was added dropwise to HSA in saline solution with continuous mixing. All reaction mixtures had a constant final volume, adjusted with the required amount of saline and/or H₂O (or other buffer) prior to the addition of ethanol, and then were incubated at room temperature for 24 h, with occasional mixing.

The effect of different molar ratios of paclitaxel and HSA on the binding of paclitaxel to HSA was determined in aqueous ethanol solution (20%, v/v) by varying the concentration of HSA at a constant amount of paclitaxel (50, 100 or 200 μg/ml). The study also evaluated the effect of different concentrations of ethanol (2, 3, 4, 5, 6, 7, 8, 10, and 15% v/v), at room temperature.

Reagent Preparation

1. A 10-ml stock solution of paclitaxel (5 mg/ml in ethanol) was prepared in a small HPLC vial and designated the 5 Ptx stock solution. Other Ptx stock solutions were also prepared when required.
2. The commercial HSA stock solution (200 mg/ml) was used.
3. A 500-ml normal saline stock solution (0.85% NaCl) was used.
   Procedure
1. Different amounts of 200 HSA stock solution (200 mg/ml) were added to each test tube, according to the scheme in Table 3.
2. Saline solution was added to each tube according to the scheme in Table 4 such that after the addition of paclitaxel and ethanol the final volume was 2 ml. Saline in water could also be added to the test tubes so that all the test tubes have substantially identical sodium chloride concentrations.
3. Based on the final concentration of paclitaxel and ethanol required in each reaction mixture, different Ptx/EtOH solutions consisting of paclitaxel (in ethanol) and additional ethanol (supplemented to give the required final ethanol concentration) were prepared in a separate set of test tubes according to the scheme in Table 5.
4. An amount of the Paclitaxel/EtOH solution was added dropwise (20 to 50 μl a drop) to the test tube containing HSA and saline according to Table 5 (refer to the last column), while vortexing to avoid the denaturation of HSA by alcohol.
5. The test tubes were covered with a piece of parafilm or in stoppered serum bottles and then incubated at room temperature, with occasional shaking (2 to 3 times).
6. At 0 hr, 3 hr, and/or 18/24 hr of incubation, the samples were observed qualitatively for precipitate formation.
7. At the end of the 18-24 hr incubation period, the reaction mixtures were analyzed for turbidity at 600 nm using a Shimadzu 160U UV/visible spectrophotometer (NBI Track #F1174).
8. The reaction mixtures were transferred to 1.5-ml Eppendorf tubes, centrifuged at 16.000×g for 10 min in a IEC Centra-MP4 microfuge (NBI Track #2078). A sample of the supernatant was saved for analysis of total Paclitaxel (bound and free) and protein contents. The supernatants were then transferred to Microcon 10 (Amicon, Oakville, ON) filtration units and centrifuged again at 16,000×g for 15 min in a IEC Centra-MP4 microfuge (NBI Track #2078).
9. The ultrafiltrate fraction of each reaction mixture was transferred to a 1.5-ml Eppendorf tube, and sent for analysis of free Paclitaxel by ELISA and/or analyzed by reverse-phase HPLC or LSC.
10. The amount of paclitaxel binding was estimated as the difference between total paclitaxel in the reaction mixture and free paclitaxel in the ultrafiltrate fraction.
11. Analytical methods. Ptx binding is analyzed by liquid scintillation counting (LSC) for formulation mixtures spiked with radioactive Ptx. This technique enables the quantitation of soluble Ptx in the free form as well as in the HSA-bound form, after fractionation by ultrafiltration using a 10-Kd cutoff Microcon UF device. Non-radioactive Ptx/HSA formulation mixtures are analyzed by ELISA, or reverse phase HPLC, after extraction of Ptx from HSA according to a procedure by Sharma et al. (1994) J. Chromatography B. 655: 315-319. This method enables the detection of Ptx degradation products, during storage. The biochemical stability of HSA will be analyzed by SDS-PAGE under reduced and non-reduced conditions.

Definition of Sample Analysis Terminology.
R: Refers to the analysis of total Ptx in the formulation mixtures which includes Ptx in the soluble and insoluble form, and is based on the initial concentration of Ptx in the reaction mixtures, before the centrifugation.
S: Refers to the total soluble Ptx in the free form and HSA-bound form. It is obtained after the centrifugation of the reaction mixtures to remove any precipitable Ptx.

F: Refers to the quantitation of free Ptx, obtained the ultrafiltrate through a 10K UF device.

TABLE 3
Paclitaxel/HSA molar ratio study: Experimental design
for the amount of HSA required in the reaction mixtures
to obtain different Paclitaxel:HSA molar ratios.

| | | Final Conc. | HSA stock solution | |
|---|---|---|---|---|
| | Conc. of | of | | Amount |
| Ptx:HSA | Ptx | HSA | Conc. | per reaction |
| molar ratio | (μg/ml) | (mg/ml) | (mg/ml) | (μl) |
| 1:0.5 | 200 | 7.8 | 200 | 78.0 |
| 1:1 | 200 | 15.6 | 200 | 155.6 |
| 1:2 | 200 | 31.2 | 200 | 311.9 |
| 1:5 | 200 | 62.4 | 200 | 799.8 |
| 1:10 | 200 | 156 | 200 | 1559.5 |
| 1:0.5 | 100 | 3.9 | 200 | 39.0 |
| 1:1 | 100 | 7.8 | 200 | 78.0 |
| 1:2 | 100 | 15.6 | 200 | 155.6 |
| 1:5 | 100 | 31.2 | 200 | 311.9 |
| 1:10 | 100 | 62.4 | 200 | 799.8 |
| 1:0.5 | 50 | 1.95 | 200 | 19.5 |
| 1:1 | 50 | 3.9 | 200 | 39.0 |
| 1:2 | 50 | 7.8 | 200 | 78.0 |
| 1:5 | 50 | 15.6 | 200 | 155.6 |
| 1:10 | 50 | 31.2 | 200 | 311.9 |

Ptx, paclitaxel.

TABLE 4
Paclitaxel/HSA molar ratio study: Experimental design for the amount
of saline required in the reaction mixtures at different concentrations
of ethanol.

| | Fixed | | | | | |
|---|---|---|---|---|---|---|
| | Conc. of | Amount of saline per reaction mixture (μl) | | | | |
| Ptx:HSA | Ptx | 20% | 15% | 10% | 5% | 2% |
| molar ratio | (μg/ml) | EtOH | EtOH | EtOH | EtOH | EtOH |
| 1:0.5 | 200 | 1522.0 | 1622.0 | 1722.0 | 1822.0 | 1882.0 |
| 1:1 | 200 | 1444.4 | 1544.4 | 1644.4 | 1744.4 | 1804.4 |
| 1:2 | 200 | 1288.1 | 1388.1 | 1488.1 | 1588.1 | 1648.1 |
| 1:5 | 200 | 820.2 | 920.2 | 1020.2 | 1120.2 | 1180.2 |
| 1:10 | 200 | 40.5 | 140.5 | 240.5 | 340.5 | 400.5 |
| 1:0.5 | 100 | 1561.0 | 1661.0 | 1761.0 | 1861.0 | 1921.0 |
| 1:1 | 100 | 1522.0 | 1622.0 | 1722.0 | 1822.0 | 1882.0 |
| 1:2 | 100 | 1444.4 | 1544.4 | 1644.4 | 1744.4 | 1804.4 |
| 1:5 | 100 | 1288.1 | 1388.1 | 1488.1 | 1588.1 | 1648.1 |
| 1:10 | 100 | 820.2 | 920.2 | 1020.2 | 1120.2 | 1180.2 |
| 1:0.5 | 50 | 1580.5 | 1680.5 | 1780.5 | 1880.5 | 1940.5 |
| 1:1 | 50 | 1561.0 | 1661.0 | 1761.0 | 1861.0 | 1921.0 |
| 1:2 | 50 | 1522.0 | 1622.0 | 1722.0 | 1822.0 | 1882.0 |
| 1:5 | 50 | 1444.4 | 1544.4 | 1644.4 | 1744.4 | 1804.4 |
| 1:10 | 50 | 1288.1 | 1388.1 | 1488.1 | 1588.1 | 1648.1 |

TABLE 5
Paclitaxel/HSA molar ratio study: Experimental design for the preparation of
Paclitaxel/EtOH solutions containing the required final amounts of Paclitaxel
in the reaction mixtures at different concentrations of ethanol.

| | | Stock Ptx | | EtOH | | Ptx/EtOH | |
|---|---|---|---|---|---|---|---|
| | | solutions | | solution | | solution | |
| | Fixed | | Amount | | Amount | | Amount |
| Final | Conc. of | | per | | per | | per |
| conc. of Ptx | EtOH | Conc. | reaction | | reaction | | reaction |
| (μg/ml) | (%) | (mg/ml) | (μl) | Conc. (%) | (μl) | Name | (μl) |
| 200 | 20 | 5.0 | 80 | 100 | 320 | 200/20 | 400 |
| 200 | 15 | 5.0 | 80 | 100 | 220 | 200/15 | 300 |
| 200 | 10 | 5.0 | 80 | 100 | 120 | 200/10 | 200 |
| 200 | 5 | 5.0 | 80 | 100 | 20 | 200/5 | 100 |
| 200 | 2 | 10.0 | 40 | 100 | 0 | 200/2 | 40 |
| 100 | 20 | 2.5 | 80 | 100 | 320 | 100/20 | 400 |
| 100 | 15 | 2.5 | 80 | 100 | 220 | 100/15 | 300 |
| 100 | 10 | 2.5 | 80 | 100 | 120 | 100/10 | 200 |
| 100 | 5 | 2.5 | 80 | 100 | 20 | 100/5 | 100 |
| 100 | 2 | 5.0 | 40 | 100 | 0 | 100/2 | 40 |
| 50 | 20 | 1.25 | 80 | 100 | 320 | 200/20 | 400 |
| 50 | 15 | 1.25 | 80 | 100 | 220 | 50/15 | 300 |
| 50 | 10 | 1.25 | 80 | 100 | 120 | 50/10 | 200 |
| 50 | 5 | 1.25 | 80 | 100 | 20 | 50/5 | 100 |
| 50 | 2 | 2.50 | 40 | 100 | 0 | 50/2 | 40 |

Results

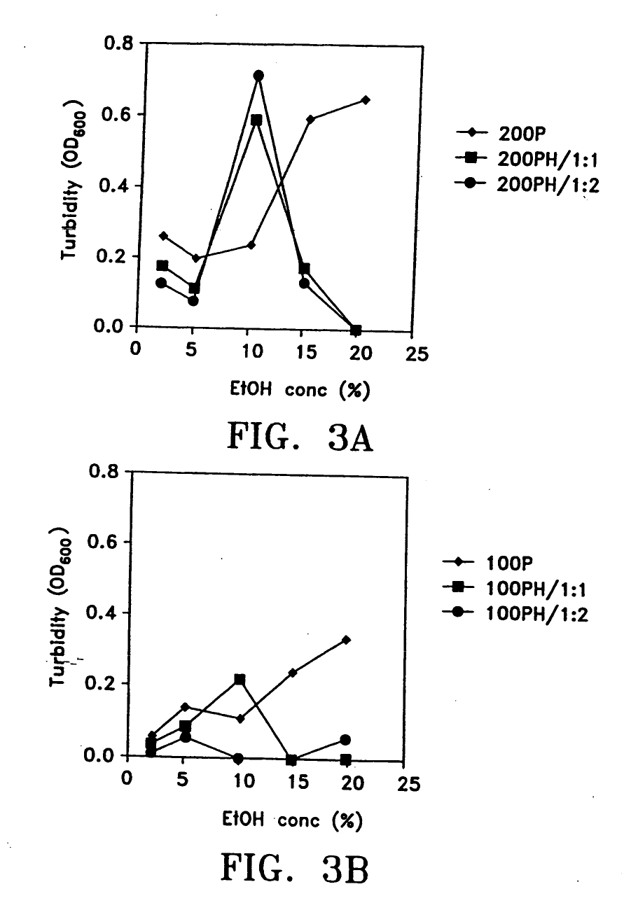

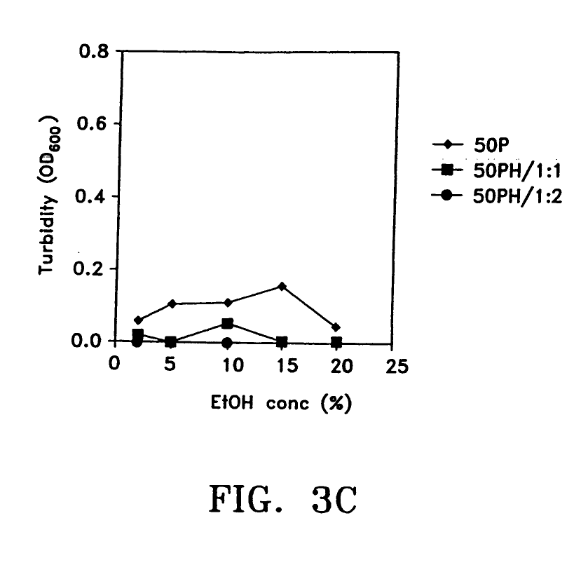

Molar ratio studies were carried out at three fixed concentrations of paclitaxel (50, 100 and 200 mg/ml) in aqueous ethanol solutions of 20% or less. To obtain the different molar ratios, the amount of HSA was varied. Although the paclitaxel concentrations of 100 and 200 mg/ml were not considered optimal, on the basis of paclitaxel solubility alone as described in section 1. C. above, they were still investigated in the presence of HSA. The mixtures were analyzed qualitatively (visual observation) for the formation of precipitates, and quantitatively by measuring the turbidity at 600 nm. FIG. 3A shows the effect of ethanol concentration at different molar ratios of paclitaxel and HSA, with the paclitaxel concentration fixed at 200 mg/ml. The results showed a reduction in the turbidity of the mixtures at lower ethanol concentration (5%), and there was no physical evidence of paclitaxel particulates sticking to the sides of the test tubes. Similarly, a reduced turbidity was observed at 20% ethanol. Without wishing to be bound by any particular theory, the inventors suggest that this reduction in turbidity may be due in part to the removal of paclitaxel from solution in the form of glass-bound insoluble small crystals. The high turbidity associated with 10% ethanol was characteristic of this condition, even in the control paclitaxel solution. This is presumably due to the more homogenous, milky appearance of the paclitaxel solution at this concentration of ethanol. On the basis of the physical appearance of the mixtures, lower ethanol concentrations (2-5%) resulted in increased solubility of paclitaxel than did higher ethanol concentrations (10-15%). This effect was dependent on HSA, since the control solutions lacking this protein, turned turbid under the experimental conditions. FIG. 3B shows a repeated experiment at 2 and 5% ethanol, using paclitaxel and HSA at molar ratios of 1:1 and 1:2.

Estimation of paclitaxel binding was carried out using two approaches. In the first method, unbound paclitaxel obtained in the 10-kDa cutoff ultrafiltration fraction was analyzed by reverse phase HPLC. The amount of bound paclitaxel was then estimated by subtracting free paclitaxel from the total paclitaxel added to the reaction mixture. An important assumption made was that no paclitaxel was binding to the membrane and no precipitable material was present prior to the filtration step. Both of these conditions were satisfied by filtering a known amount of paclitaxel as a control and estimating recovery; and by centrifuging before the ultrafiltration step. No detectable level of free paclitaxel was observed from reaction mixtures containing paclitaxel and HSA in molar ratios of 1:1 and 1:2 in the presence of 2% and 5% ethanol (Data not shown). This implies that at these molar ratios, and at a fixed paclitaxel concentration of 50 and 100 μg/ml nearly 100% binding was obtained. The detection limit of the assay was around 10 μg/ml, which is not sensitive enough to detect lower concentrations of paclitaxel. Consequently, these results need be interpreted with some caution.

The second approach was based on the quantitation of both free and total paclitaxel by ELISA, as shown in Table 6. Advantage was taken of a finding that showed that HSA did not interfere with the analysis of paclitaxel by ELISA. It can be concluded that in most cases the amount of total paclitaxel estimated by ELISA was greater than 84% the expected amount. Greater than 85% binding was estimated, when paclitaxel was added to HSA at molar ratios of 1:1 and 1:2, with paclitaxel fixed at concentrations of either 50 or 100 mg/ml.

TABLE 6
Estimation of paclitaxel binding to HSA at two different molar ratios and dilute
ethanol concentrations.

| | Ptx | | | | | | |
|---|---|---|---|---|---|---|---|
| | conc. | HSA | | Ethanol | | | |
| | (mg/ | conc. | | conc. | Turbidity | Free Ptx | Estimated |
| Sample name | ml) | (mg/ml) | Molar ratio | (%) | (OD₆₀₀) | (mg/ml) | % binding |
| P-200 | 200 | NA | NA | 5 | 0.306 | ND | ND |
| PH-200/1 | 200 | 15.6 | 1:1 | 5 | 0.101 | 9.7 | 95.2 |
| PH-200/1 | 200 | 15.6 | 1:1 | 2 | 0.122 | 8.2 | 95.9 |
| PH-200/2 | 200 | 31.2 | 1:2 | 5 | 0.096 | 18.7 | 90.6 |
| PH-200/2 | 200 | 31.2 | 1:2 | 2 | 0.077 | 19.3 | 90.3 |
| P-100 | 100 | NA | NA | 5 | 0.101 | ND | ND |
| PH-100/1 | 100 | 7.8 | 1:1 | 5 | 0.077 | >6 | <94 |
| PH-100/1 | 100 | 7.8 | 1:1 | 2 | 0.057 | 5.2 | 94.8 |
| PH-100/2 | 100 | 15.6 | 1:2 | 5 | 0.081 | >6 | <94 |
| PH-100/2 | 100 | 15.6 | 1:2 | 2 | 0.064 | 7.5 | 85.1 |
| P-50 | 50 | NA | NA | 5 | 0.125 | ND | ND |
| PH-50/1 | 50 | 3.9 | 1:1 | 5 | 0.004 | >5 | <92 |
| PH-50/1 | 50 | 3.9 | 1:1 | 2 | 0.017 | >5 | <92 |
| PH-50/2 | 50 | 7.8 | 1:2 | 5 | 0.012 | 1.9 | <96.2 |
| PH-50/2 | 50 | 7.8 | 1:2 | 2 | 0.020 | 3.5 | <93.1 |

(1) Three paclitaxel stock solutions were prepared with concentrations of 5, 2.5 and 1.25 mg/ml in ethanol for the 200, 100 and 50 mg/ml paclitaxel final reaction mixture samples. The last two are prepared by two-fold dilution of the 5 mg/ml stock.
(2) Paclitaxel and ethanol were added to HSA in the reaction test tube.
(3) Background A₆₀₀ value for HSA was subtracted from the turbidity values.
(4) Sample names: P-200 (the control sample containing 200 mg/ml paclitaxel); and PH-200/2 (the mixture containing paclitaxel at a fixed concentration of 200 μg/ml with HSA at a molar ratio of 1:2)
(5) NA: not applicable; ND: not determined.

1.F. Paclitaxel and HSA Binding: Effect of Non-defatted or Defatted HSA

The effect of defatting HSA on the binding of paclitaxel to HSA was determined. Commercial preparations of HSA are partly stripped of the fatty acids that are otherwise bound to HSA under physiological conditions. It is hypothesized that the residual fatty acids may be interfering with the binding of paclitaxel to HSA, although evidence suggests that long chain fatty acid binding sites are separate from small organic compounds binding sites. Carter et al. (1994). In addition, the effect can be determined of adding fatty acids (oleate, palmitate or stearate) to the reaction mixture to fully charge HSA, with the assumption that the fatty acid may serve as the linker in the binding of paclitaxel to HSA or modify the conformation of HSA to facilitate paclitaxel binding. Different molar ratios were evaluated.

HSA is induced to an expanded form by acid treatment to a pH of about 3.1 to about 3.4, then passed through a charcoal pad for the removal of fatty acids. Alternatively, fatty acids can be solvent-extracted from HSA, before reacting HSA with paclitaxel.

Experimental Procedure

Defatted HSA was prepared by acidifying the commercial HSA solution (Desert Biologicals, Phoenix, Ariz.) with 0.1-1 N HCl to a pH of 3.1-3.4, followed by filtration through a charcoal pad (Celluloco, Fresno, Calif.) and readjustment of pH to 7.0 with 2 N NaOH. Four different preparations of HSA were collected at different steps of the defatting of the commercial HSA solution as follows:
HSA solution A: a pH 7.1 non-defatted HSA solution
HSA solution B: a pH 3.3 non-defatted HSA solution
HSA solution C: a pH 7.1 defatted HSA solution
HSA solution D: a pH 3.7 defatted HSA solution

Both non-defatted and defatted HSA (solutions A and C, pH 7.1), were used to bind paclitaxel at a molar ratio of 1:1 in aqueous ethanol solutions of 2 and 5%. In this experiment, two concentrations of paclitaxel were tested: 50 and 100 μg/mL, and the reaction mixtures were brought up to a constant volume (2 mL) with a physiological saline solution. The results in Table 7 showed that at 50 μg/mL, complete binding of paclitaxel to either non-defatted or defatted HSA was achieved, in aqueous ethanol solution of 5%. However, in 2% ethanol, a lower recovery of bound paclitaxel was observed: 73+15% and 89+24% for defatted and non-defatted, respectively. The standard variation was large in this case, making it difficult to establish the effect of the two HSA preparations on the binding of paclitaxel in a 2% aqueous ethanol solution. At 100 μg/mL paclitaxel, the highest binding was obtained only with defatted HSA in 5% ethanol.

TABLE 7
Determination of the effect of defatted HSA on the binding of paclitaxel in 2 and
5% aqueous ethanol solutions.

| | Ptx | | | | | | |
|---|---|---|---|---|---|---|---|
| | conc. | HSA | | EtOH | | Total | Bound |
| | (μg/ | conc. | | conc. | Free Ptx | Ptx | Ptx |
| Sample name | mL) | (mg/mL) | Molar ratio | (%) | (μg/mL) | (μg/mL) | (%) |
| PH-23-5.50/1-8A | 50 | 3.9 | 1:1 | 5 | 0.19 | 56.8 | 113.2 |
| PH-23-5.50/1-8C | 50 | 3.9 | 1:1 | 5 | 0.43 | 57.0 | 113.0 |
| PH-23-2.50/1-8A | 50 | 3.9 | 1:1 | 2 | 0.20 | 44.8 | 89.3 |
| PH-23-2.50/1-8C | 50 | 3.9 | 1:1 | 2 | 0.41 | 36.7 | 72.6 |
| PH-23-5.100/1-8A | 100 | 7.8 | 1:1 | 5 | 0.09 | 57.5 | 57.4 |
| PH-23-5.100/1-8C | 100 | 7.8 | 1:1 | 5 | 0.80 | 99.1 | 98.3 |
| PH-23-2.100/1-8A | 100 | 7.8 | 1:1 | 2 | 0.44 | 76.3 | 75.8 |
| PH-23-2.100/1-8C | 100 | 7.8 | 1:1 | 2 | 1.01 | 73.4 | 72.4 |

Note:
The reaction mixtures were brought up to a constant volume of 2 mL with a saline solution. Conc., concentration.
HSA solutions: A for pH 7.1 non-defatted HSA solution; C for pH 7.1 defatted HSA solution.

1.G. Analysis of the Effect of Ethanol Concentration on the Binding of Ptx to Different Types of HSA in Saline.

In the process of preparing defatted HSA, 4 types of HSA have been defined. In this study, we evaluated the effect of ethanol concentration on the binding of Ptx (200 μg/ml) to defatted HSA at pH 3.5 and 7.0 and undefatted HSA at pH 3.5 and pH7.0 at a molar ratio of 1:1. The stability of the formulations was evaluated after day storage at room temperature.
1. Materials
   1.1 Different HSA solutions (10% w/v).
   Prepare at least 13 mL each.
      10% HSA-A, neutral pH undefatted (pH 6.8-7.0).
      10% HSA-B, acidic pH undefatted (pH 3.1-3.3).
      10% HSA-C, neutral pH defatted (pH 6.8-7.0).
      10% HSA-D, acidic pH defatted (pH 3.1-3.3).
   1.2 Two radioactive solutions (5 mini and 10 mg/mL paclitaxel in dehydrated EtOH containing hot Ptx at 1/200 dilution).
   1.3 Two saline solutions, pH adjusted to 3.3 and 7.0 with dilute phosphoric acid.
2. Procedure
   2.1 Preparation of 0.85% saline solution for the 4% ethanol concentration reaction mixtures.
      Adjust the pH of the saline to the pH of the reaction (i.e. pH 3.3 and 7.0). Note that for the neutral pH reaction, the saline solution needs not be adjusted.
   2.2 Preparation of Ptx sub-stock solutions for the different final ethanol concentrations.
      2.2.1 Prepare 8 Ptx/EtOH sub-stock solutions by mixing the radioactive Ptx stock solutions with dehydrated ethanol as per Table 1.
      2.2.2 Add the indicated amount of the mixture of to the solution of saline and HSA for the corresponding ethanol concentration as per Table 2.
   2.3 Formulation reaction mixture preparation.
      2.3.1 Formulation conditions:
         2.3.1.1 Ptx concentration: 200 μg/mL.
         2.3.1.2 Molar ratio: varying from 1:1.
         2.3.1.3 HSA concentration: 15.6 mg/mL.
         2.3.1.4 Ethanol concentration: varying from 2 to 20% (v/v).
         2.3.1.5 Binding solution: saline (final concentration of 0.55% NaCl).
      2.3.2 For each of the five HSA types, set up 8 tubes in triplicate for the 8 different concentrations of ethanol (2, 4, 5, 6, 8, 10, 15 and 20%), for a total of 4×8×3=96 tubes.
      2.3.3 To appropriate tubes add 1288 μL of the right pH saline solution in triplicates as per Table 1.
      2.3.4 Then add to each tube 312 μL of the appropriate 10% HSA solution as per Table 1.
      2.3.5 With constant mixing, slowly add the Ptx/EtOH sub-stock solutions as per Table 2.
   2.4 Sample analysis.
      2.4.1 Analyze the recovery and binding by LSC.
         2.4.1.1 Day 0: R, S and F.
         2.4.1.2 Day 1: Sand F.

2.4.2 Collect data for processing and analysis by excel.

TABLE 8
Ethanol concentration study: Experimental design for the amount of HSA. saline
and WFI (water for injection) required in the reaction mixtures to obtain different
concentrations of ethanol.

| | | | | HSA, saline and WFI additions for the | | |
|---|---|---|---|---|---|---|
| Reaction condition | | | | different ethanol concentrations | | |
| | | | Concentrated | | | Required |
| | | | HSA | Required | Required | amount of |
| | Final | Final | stock | amount of | amount of | WFI to QS to |
| EtOH | conc. of | conc. of | solution | HSA per | saline per | 1.6 mL per |
| conc. | Ptx | HSA | conc. | 2-mL | 2-mL reaction | 2-mL reaction |
| (%) | (μg/mL) | (mg/mL) | (mg/mL)⁽¹⁾ | reaction (μL) | (μL) | (μL) |
| 2 | 200 | 15.6 | 100 | 312 | 1288 | 360 |
| 4 | 200 | 15.6 | 100 | 312 | 1288 | 320 |
| 5 | 200 | 15.6 | 100 | 312 | 1288 | 300 |
| 6 | 200 | 15.6 | 100 | 312 | 1288 | 280 |
| 8 | 200 | 15.6 | 100 | 312 | 1288 | 240 |
| 10 | 200 | 15.6 | 100 | 312 | 1288 | 200 |
| 15 | 200 | 15.6 | 100 | 312 | 1288 | 100 |
| 20 | 200 | 15.6 | 100 | 312 | 1288 | 0 |

⁽¹⁾The 4 HSA stock solutions concentration is 100 mg/mL (10%, w/v).

TABLE 9
Ethanol concentration study: Experimental design for the preparation of
Ptx/Ethanol sub-stock solutions for the different reaction mixture ethanol
concentrations.

| | | | Ptx/EtOH sub-stock solutions for different | | | |
|---|---|---|---|---|---|---|
| | | | ethanol concentrations | | | |
| Reaction | | | | Required | Required | Required |
| condition | | | Conc. of | amount of | amount of | amount of |
| | Final | Final | Ptx stock | Ptx per | EtOH per | Ptx/EtOH |
| | conc. of | conc. of | solution | 2-mL | 2-mL | mixture per |
| EtOH conc. | Ptx | HSA | conc. | reaction | reaction | 2-mL reaction |
| (%) | (μg/mL) | (mg/mL) | (mg/mL)⁽¹⁾ | (μL) | (μL) | (μL) |
| 2 | 200 | 15.6 | 10 | 40 | 0 | 40 |
| 4 | 200 | 15.6 | 5 | 80 | 0 | 80 |
| 5 | 200 | 15.6 | 5 | 80 | 20 | 100 |
| 6 | 200 | 15.6 | 5 | 80 | 40 | 120 |
| 8 | 200 | 15.6 | 5 | 80 | 60 | 160 |
| 10 | 200 | 15.6 | 5 | 80 | 120 | 200 |
| 15 | 200 | 15.6 | 5 | 80 | 220 | 300 |
| 20 | 200 | 15.6 | 5 | 80 | 320 | 400 |

⁽¹⁾Two Ptx stock solutions (5 mg/mL and 10 mg/mL) were prepared in absolute ethanol. Conc., concentration.

Results

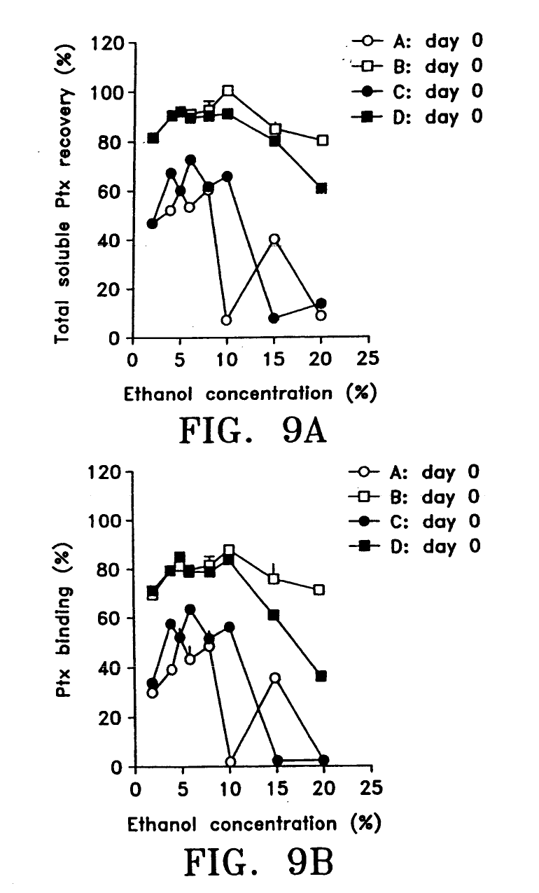

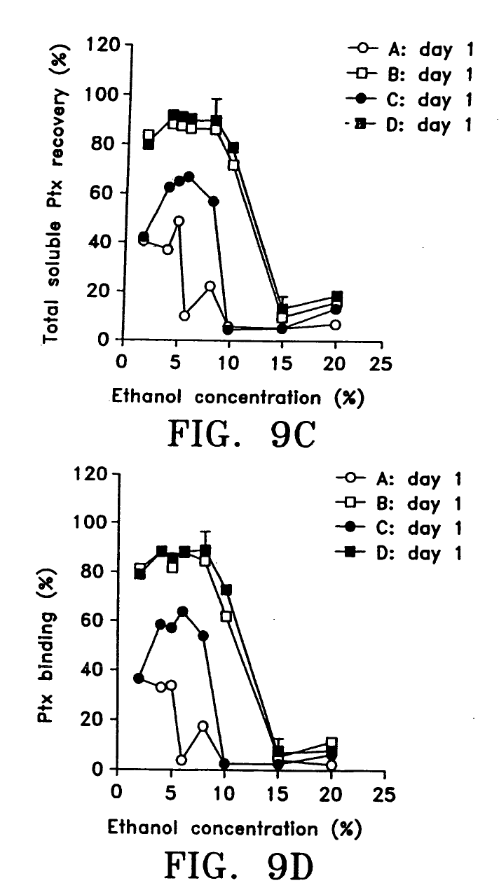

As previously shown by ELISA, the radioactive study results indicated high binding of Ptx to HSA (defatted and undefatted) occurred at acidic pH (FIG. 9, panels A and B). Panel A shows the recovery of total soluble Ptx and B shows the binding at day 0. The combined effect of low ethanol concentration (below 10% v/v) and low pH resulted in a more stable binding and high recovery after 1 day storage (FIG. 9, panels C and D). We also analyzed the recovery and binding at 15% and 20% at different molar ratios and different concentrations of Ptx (10, 100 and 200 μg/ml) after 1-day storage (Table 10). Quantitation of Ptx binding by ELISA in reaction mixtures containing 15% and 20% ethanol and different Ptx:HSA molar ratios. The results showed that despite the observed reduced turbidity of the reaction mixtures after 16 h-24 h of incubation at room temperature, and at ethanol concentrations greater than 10%, the recovery of total soluble Ptx, as well as the binding of Ptx to HSA, was poor. Suggesting that the high ethanol concentration conditions were not suitable formulation conditions.

TABLE 10
Quantitation of soluble Ptx recovery and binding to HSA at
different molar ratios in saline solutions containing 15% and
20% ethanol, after 1-day storage at 23 ° C.

| | | 20% | 20% | 15% | 15% |
|---|---|---|---|---|---|
| | | ethanol | ethanol | ethanol | ethanol |
| HSA | | Total | Estimated | Total | Estimated |
| excess | | soluble Ptx | % | soluble Ptx | % |
| molar | Ptx conc. | Recovery | Bound | Recovery | Bound |
| amount | (μg/mL) | (%) | Ptx | (%) | Ptx |
| 0.5 | 200 | 4.0 | −0.7 | 3.1 | 1.6 |
| 1 | 200 | 4.5 | −3.1 | 2.7 | 1.0 |
| 2 | 200 | 7.1 | −2.5 | 2.6 | −0.1 |
| 5 | 200 | 10.9 | 0.4 | 3.4 | 0.3 |
| 10 | 200 | 17.8 | 4.8 | 2.7 | −1.8 |
| 0.5 | 100 | 11.4 | −1.7 | 4.1 | 1.3 |
| 1 | 100 | 13.1 | 2.9 | 4.2 | 1.1 |
| 2 | 100 | 11.5 | 1.4 | 5.5 | 2.0 |
| 5 | 100 | 11.0 | −2.2 | 6.2 | −0.8 |
| 10 | 100 | 20.9 | 4.6 | 8.0 | −4.8 |
| 0.5 | 50 | 21.2 | 4.5 | 22.4 | 5.8 |
| 1 | 50 | 35.2 | 18.0 | 15.3 | 3.1 |
| 2 | 50 | 34.0 | 16.3 | 9.5 | 2.2 |
| 5 | 50 | 30.4 | 11.9 | 16.3 | 6.0 |
| 10 | 50 | 47.9 | 30.1 | 10.1 | −1.1 |

1. H. Paclitaxel and HSA Binding: Effect of pH

The pH of a protein solution affects the charge distribution on the protein, consequently affecting its solubility properties as well as its interaction with other molecules. Non-defatted and defatted HSA have a pI of 4.7 and 5.3, respectively. Carter et al. (1994). As evident from the experiment with acidic preparations of HSA, clear solutions of paclitaxel/HSA could be prepared in acidic media. Therefore, a more systematic analysis of the effect of pH on the binding of paclitaxel to HSA was necessary.

The effect of pH on the binding of paclitaxel to HSA can be analyzed in phosphate vehicle adjusted with acid (e.g., phosphoric acid, 0.1 M) as required, e.g. to pH values of 7, 6, 5 and 4.

As a first experiment on the effect of pH, the two acidic preparations of HSA (solutions B and D, pH 3.3 with non-defatted and 3.7 with defatted, respectively) were used to evaluate the effect of low pH on the binding of paclitaxel to either defatted and non-defatted HSA. The results, shown in Table 11 and FIG. 9, suggest that any of the conditions tested resulted in a complete binding of paclitaxel to HSA, irrespective of the HSA fat content, and the reaction mixture paclitaxel concentration (50 or 100 μg/mL). The pH of these reaction mixtures was around 4.3.

TABLE 11
Determination of the effect of acidic preparations of defatted
and non-defatted HSA on the binding of paclitaxel in 2 and 5%
aqueous ethanol solutions.

| | Ptx | HSA | | EtOH | Free | Total | Bound |
|---|---|---|---|---|---|---|---|
| | conc. | conc. | Molar | conc. | Ptx | Ptx | Ptx |
| Sample name | (μg/mL) | (mg/mL) | ratio | (%) | (μg/mL) | (μg/mL) | (%) |
| PH-23-5.50/1-8B | 50 | 3.9 | 1:1 | 5 | 0.54 | 60.0 | 118.9 |
| PH-23-5.50/1-8B | 50 | 3.9 | 1:1 | 5 | 0.16 | 59.4 | 118.9 |
| PH-23-2.50/1-8B | 50 | 3.9 | 1:1 | 2 | 0.32 | 57.4 | 114.1 |
| PH-23-2.50/1-8D | 50 | 3.9 | 1:1 | 2 | 0.69 | 53.1 | 104.7 |
| PH-23-5.100/1-8B | 100 | 7.8 | 1:1 | 5 | 1.51 | 108.0 | 106.5 |
| PH-23-5.100/1-8D | 100 | 7.8 | 1:1 | 5 | 1.25 | 112.5 | 111.3 |
| PH-23-2.100/1-8B | 100 | 7.8 | 1:1 | 2 | 0.92 | 121.5 | 120.6 |
| PH-23-2.100/1-8D | 100 | 7.8 | 1:1 | 2 | 1.45 | 123.5 | 122.1 |

Note:
The reaction mixtures were brought up to a constant volume of 2 mL with a saline solution. Conc., concentration
HSA solutions: B for pH 3.3 non-defatted HSA solution; D for pH 3.7 defatted HSA solution

Experimental Procedure

The experimental procedure is described below. All reaction mixtures contained 5% ethanol and paclitaxel/HSA in a molar ratio of 1:1. The pH range of 2.6 to 7.2 was obtained by preparing McIlvaine buffer solutions. These consist of adding different proportions of 0.1 M citric acid and 0.2 M Na₂HPO₄ solutions. Dawson et al. (1986) Data for Biochemical Research, 3 ed., Oxford Science Publications, Oxford. Britain, p. 427.

The following pH values were analyzed: 2.6, 3.0, 3.4, 4.0, 4.4, 5.0, 5.4, 6.0, 6.6 and 7.2. The experiment was conducted at room temperature with paclitaxel/HSA used at a molar ratio of 1:1 at fixed paclitaxel concentrations of 200, 300 and 400 μg/mL. The reaction mixture ethanol concentration was 5% (v/v). The commercial non-defatted HSA stock (200 mg/mL) and defatted HSA (pH 7, 165.4 mg/mL) were used.

Reagents Preparation
1. Three ml-stock solutions of paclitaxel (5, 7.5 and 10 mg/mL in ethanol) were prepared in small HPLC vials. These were referred as the 5 Ptx, 7.5 Ptx and 10 Ptx stock solutions.
2. The commercial HSA stock solution (200 mg/mL) was used in duplicates for all three paclitaxel concentrations; and the defatted HSA (165.4 mg/mL) was used only for the 300 μg/mL paclitaxel.
3. Two stock solutions of 0.1 M citric acid monohydrate USP-grade (MW: 210.14) and Na₂HPO₄, 7H₂O (MW: 268.07) was prepared as follows:
3.1 Dissolve 10.51 g of citric acid monohydrate in 500 mL of water to make a 0.1 M solution of citric acid
3.2 Dissolve 26.81 g of Na₂HPO₄, 7H₂O in 500 mL of water to make a 0.2 M solution of Na₂HPO₄.

4. McIlvaine buffer solutions (50 mL each) of different pH were prepared by mixing×mL of 0.1 M citric acid with y mL of Na₂HPO₄ according to Table 12A.

TABLE 12A
Paclitaxel/HSA pH experiment: Experimental design
for the preparation of McIlvaine buffer solutions of different pH.

| | x mL 0.1 M | y mL 0.2 M |
|---|---|---|
| pH | Citric acid | Na₂HPO₄ |
| 2.6 | 44.55 | 5.45 |
| 3.0 | 39.73 | 10.27 |
| 3.4 | 35.75 | 14.25 |
| 4.0 | 30.73 | 19.27 |
| 4.4 | 27.95 | 22.05 |
| 5.0 | 24.25 | 25.75 |
| 5.4 | 22.13 | 27.87 |
| 6.0 | 18.43 | 31.57 |
| 6.6 | 13.63 | 36.37 |
| 7.2 | 6.53 | 43.47 |

Procedure
1. Different amounts of HSA stock solution were added to each test tube, according to the scheme in Table 12B.
2. McIlvaine solution was added to the appropriate tube according to the scheme in Table 12B such that after the addition of paclitaxel and ethanol the final volume was 2 mL.
3. Based on the final concentrations of paclitaxel and ethanol in each reaction mixture, different paclitaxel/EtOH solutions consisting of paclitaxel (80 μL, in ethanol) and additional ethanol (20 μL supplemented to give the final ethanol concentration of 5%) was prepared in a separate set of test tubes according to the scheme in Table 12C.
4. 100 μL amount of the paclitaxel/EtOH solution was added dropwise (about 8 to 20 μl each drop) to the test tube containing HSA and vehicle solution according to Table 12C (refer to last column), while vortexing to avoid the denaturation of HSA by ethanol.
5. The test tubes were covered with a parafilm and then incubated at room temperature, with occasional shaking. Alternatively, the mixtures were prepared in stoppered serum vials.
6. At 0 h, 3 h and/or 18-24 h of incubation the samples was observed qualitatively for precipitate formation.
7. At the end of the 18-24-h incubation period, the reaction mixtures were analyzed for turbidity at 600 nm using a Shimadzu 160U UV/visible spectrophotometer (NBI Track # F 1174).
8. The reaction mixtures were transferred to 1.5-mL Eppendorf tubes, centrifuged at 16,000×g for 10 nin, in a IEC Centra-MP4 microfuge (NBI Track # 2078). A sample of the supernatant was saved for the analysis of total paclitaxel (bound and free) and protein contents. The supernatants were be transferred to Microcon 10 (Amicon, Oakville, Ca.) filtration units and centrifuged again at 14,000×g for 15 min in a IEC Centra-MP4 centrifuge (NBI track #2078).
9. The ultrafiltrate fraction of each reaction mixture was transferred to a 1.5-mL Eppendorf tube, and sent for the analysis of free paclitaxel by ELISA and/or analyzed by reverse-phase HPLC.

10. The amount of paclitaxel binding was estimated as the difference between total paclitaxel in the reaction mixture and free paclitaxel in the ultrafiltrate fraction.

TABLE 12B
Paclitaxel/HSA pH study: Experimental design for the amount of HSA and
vehicle required in the reaction mixtures to obtain a 1:1 molar ratio at different
fixed concentrations of Paclitaxel.

| | | | | HSA stock | | Vehicle | Ptx/HSA |
|---|---|---|---|---|---|---|---|
| | | | | solution | | solution | solution |
| | | | | | Amount | Amount | Amount |
| Ptx:HSA | Final Ptx | Final HSA | Final EtOH | | per | per | per |
| molar | conc. | conc. | conc. | Conc. | reaction | reaction | reaction |
| ratio | (μg/mL) | (mg/mL) | (%) | (mg/mL) | (μL) | (μL) | (μL) |
| 1:1 | 400 | 3.12 | 5 | 200 | 311.2 | 1588.8 | 100 |
| 1:1 | 300 | 23.4 | 5 | 200 | 233.4 | 1666.6 | 100 |
| 1:1* | 300* | 23.4* | 5* | 200* | 282.2* | 1617.8* | 100* |
| 1:1 | 200 | 15.6 | 5 | 200 | 155.6 | 1744.4 | 100 |

*This row applies only to the defatted HSA to account for the dilution of the commercial stock solution during pH adjustments made in the preparation of this solution.

Paclitaxel/HSA solution was prepared as per Table 12C.

TABLE 12C
Paclitaxel/HSA pH study: Experimental design for the
preparation Paclitaxel/EtOH solutions containing the
required final amounts of Paclitaxel in the reaction
mixtures at different concentrations of ethanol.

| | | Stock Ptx | | EtOH | | Ptx/EtOH | |
|---|---|---|---|---|---|---|---|
| | | solutions | | solution | | solution | |
| Final | Final | | Amount | | Amount | | Amount |
| conc. of | conc. of | | per | | per | | per |
| Ptx | EtOH | Conc. | reaction | Conc. | reaction | | reaction |
| (μg/mL) | (%) | (mg/mL) | (μL) | (%) | (μL) | Name | (μL) |
| 400 | 5 | 10.0 | 80 | 100 | 20 | 400/5 | 100 |
| 300 | 5 | 7.5 | 80 | 100 | 20 | 300/5 | 100 |
| 200 | 5 | 5.0 | 80 | 100 | 20 | 200/5 | 100 |

Results

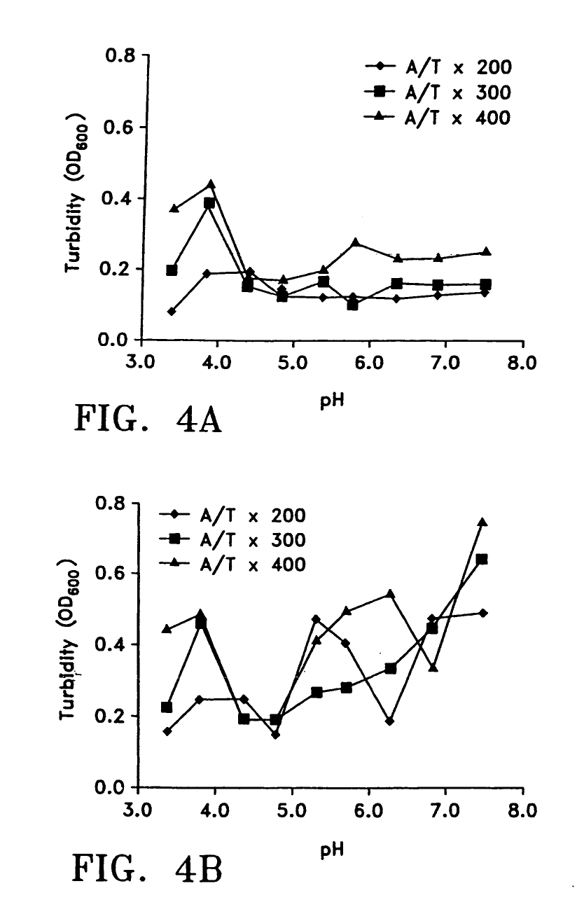

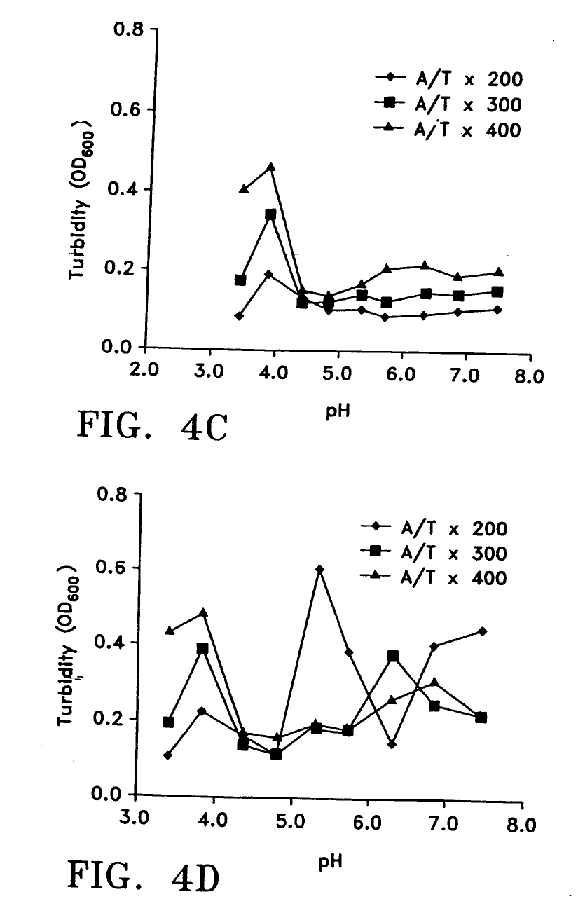

Since a complete binding of paclitaxel was observed in acidic reactions mixtures containing 100 μg/mL, there was some interest in evaluating the effect of pH on the binding of paclitaxel to non-defatted HSA at slightly higher concentrations of paclitaxel, including 200, 300 and 400 μg/mL. The reaction mixtures were prepared in glass and plastic test tubes, and incubated at room temperature. The turbidity of the mixtures was measured after 24 h and 96 h. The results obtained with the various conditions tested are shown in FIG. 4. The optimal pH for both paclitaxel binding to HSA and stability of the complex was found in the range of 4.4 to 4.8, based on the stability of the paclitaxel-HSA solution (clear solutions). Near neutral pH the stability of paclitaxel/HSA complex was poor during prolonged incubation, as the turbidity of the solutions increased with time. This effect was, unexpectedly, more pronounced in glass tubes than in plastic tubes.

A quantitative analysis of paclitaxel binding was done as previously described by estimating the amount of bound paclitaxel from the total paclitaxel content in the reaction mixtures, following the removal of insoluble paclitaxel by centrifugation and subtracting the amount of free paclitaxel present in the ultrafiltrate fraction. The ELISA data obtained after a 96-h incubation are shown in Table 13A. Complete binding of paclitaxel (200 and 300 μg/mL) was achieved at pH of 4.8 and 4.5, respectively. The exact pH for maximal binding and stability must be further determined for different concentrations of paclitaxel. Other conditions were not analyzed because of the presence of precipitated paclitaxel (turbid reactions mixtures after the 96-h incubation).

TABLE 13A
Determination of the effect of pH on the binding of paclitaxel to non-defatted
HSA in glass test tubes. after a 96-h incubation at 23° C.

| | | Ptx | HSA | | | Total | Bound |
|---|---|---|---|---|---|---|---|
| | | conc. | conc. | Molar | Free Ptx | Ptx | Ptx |
| Sample name | pH | (μg/mL) | (mg/mL) | ratio | (μg/mL) | (μg/mL) | (%) |
| PH-23-5.200/1*3.0-10A | 3.40 | 200 | 15.6 | 1:1 | 13.6 | 137 | 61.7 |
| PH-23-5.200/1*4.0-10A | 4.39 | 200 | 15.6 | 1:1 | 12.0 | 188 | 88.0 |
| PH-23-5.200/1*4.4-10A | 4.81 | 200 | 15.6 | 1:1 | 46.7 | 264 | 108.6 |
| PH-23-5.300/1*3.0-10A | 3.61 | 300 | 23.4 | 1:1 | 34.6 | 407 | 124.1 |
| PH-23-5.300/1*4.0-10A | 4.51 | 300 | 23.4 | 1:1 | 38.7 | 367 | 109.4 |
| PH-23-5.300/1*4.4-10A | 4.84 | 300 | 23.4 | 1:1 | 41.0 | 172 | 43.7 |
| PH-23-5.400/1*3.0-10A | 4.92 | 400 | 31.2 | 1:1 | 14.6 | 249 | 58.6 |

Note:
The reaction mixture were kept in glass test tubes at 23° C. for 96 h before the analysis of paclitaxel. They all contained 5% ethanol (v/v), and were brought up to a constant volume of 2 mL with an appropriate McIlvaine butter solution.
HSA solutions: A for pH 7.1 non-defatted HSA solution.

TABLE 13B
Determination of the effect of pH on the binding of paclitaxel to non-defatted
HSA in plastic test tubes. after a 96-h incubation at 23° C.

| | | Ptx | HSA | | | Total | Bound |
|---|---|---|---|---|---|---|---|
| | | conc. | conc. | Molar | Free Ptx | Ptx | Ptx |
| Sample name | pH | (μg/mL) | (mg/mL) | ratio | (μg/mL) | (μg/mL) | (%) |
| PH-23-5.200/1*3.0-10A | 3.40 | 200 | 15.6 | 1:1 | 407 | 226 | 92.7 |
| PH-23-5.200/1*4.0-10A | 4.39 | 200 | 15.6 | 1:1 | 66.7 | 220 | 76.6 |
| PH-23-5.200/1*4.4-10A | 4.81 | 200 | 15.6 | 1:1 | 27.1 | 264 | 118.4 |
| PH-23-5.300/1*3.0-10A | 3.61 | 300 | 23.4 | 1:1 | 49.0 | 454 | 135 |
| PH-23-5.300/1*4.0-10A | 4.51 | 300 | 23.4 | 1:1 | 2.9 | 292 | 96.4 |
| PH-23-5.300/1*4.4-10A | 4.84 | 300 | 23.4 | 1:1 | 13.8 | 214 | 66.7 |
| PH-23-5.400/1*3.0-10A | 4.92 | 400 | 31.2 | 1:1 | 53.9 | 136 | 20.5 |

Note:
The reaction mixtures were kept in plastic test tubes at 23° C. for 96 h before the analysis of paclitaxel. They all contained 5% ethanol (v/v), and were brought up to a constant volume of 2 mL with an appropriate McIlvaine buffer solution.
HSA solutions: A for pH 7.1 non-defatted HSA solution.

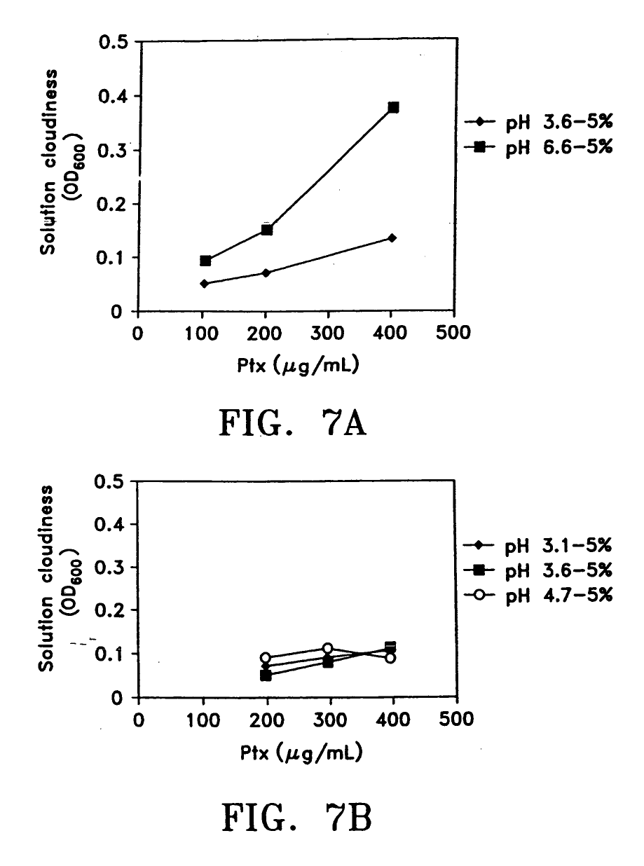

FIGS. 7A and 7B depict the effect of pH and paclitaxel concentration on the binding of paclitaxel to HSA at a molar ratio of 1:1. The reaction mixtures contained 5% ethanol and different concentrations of paclitaxel with HSA added to a molar ratio of 1:1. In FIG. 7A, the HSA preparations were undefatted HSA at neutral pH (6.6) or defatted HSA at acidic pH (3.6) in saline-based reaction mixtures. The turbidity of the solutions was measured after a 14 hr incubation at 23° C. In FIG. 7B, the HSA preparations were a neutral pH de-fatted HSA preparation in McIlvaine buffer-based reaction mixtures of varying pH: the turbidity of the solutions was measured after a 16-hr incubation at 23° C.

1.I. Effect of Acidic pH on the Storage Stability of Paclitaxel/HSA

In another experiment the effect of an acidic preparation of defatted HSA on the binding of paclitaxel from reaction mixtures of slightly higher concentrations was evaluated. The amount of bound paclitaxel was measured by ELISA following an 11-day incubation. The results shown in Table 14 suggest that at the pH of 3.6 a good recovery of bound paclitaxel could be obtained despite the prolonged incubation at room temperature. An interesting observation was that even at 400 μg/mL of paclitaxel good binding occurred. This we found to be dependent partly on the technique of addition of paclitaxel to the HSA solution, the pH, and the preparation of HSA (being a fast or expanded pH induced conformation of HSA).

TABLE 14
Determination of the effect of an acidic defatted HSA preparation
on the binding of paclitaxel in reaction mixtures of increasing
paclitaxel concentration, after a 11-day incubation at 23° C.

| | | Ptx | HSA | | Free | Total | Bound |
|---|---|---|---|---|---|---|---|
| | | conc. | conc. | Molar | Ptx | Ptx | Ptx |
| Sample name | pH | (μg/mL) | (mg/mL) | ratio | (μg/mL) | (μg/mL) | (%) |
| PH-23-5.100/1-9D | 3.60 | 100 | 7.9 | 1:1 | 0.7 | 94.23 | 93.6 |
| PH-23-5.200/1-9D | 3.60 | 200 | 15.6 | 1:1 | 0.8 | 175 | 87.1 |
| PH-23-5.400/1-9D | 3.59 | 400 | 31.2 | 1:1 | 17.0 | 416 | 99.8 |

Note:
The reaction mixtures were kept in glass test tubes at 23° C. for 11 days before the analysis of paclitaxel. All reaction mixtures contained 5% ethanol (v/v), and were brought up to a constant volume of 2 mL with a saline solution.
HSA solutions: D for pH 3.7 defatted HSA solution.

Effect of pH on the Binding of Ptx to HSA and the Stability of the Formulation

The following studies reevaluated (i) the effect of acidic and neutral pH on the recovery of soluble Ptx and binding to HSA at molar ratios of 1:1 and 1:2, as well as the formulation stability; and (ii) the pH profile of the recovery of soluble Ptx and binding to HSA-B (acidic undefatted) and HSA-D (acidic defatted) in the McIlvaine buffer system at 1:1 molar ratio.

Experiment 1. Evaluation of Ptx Binding to Different Preparations of Defatted HSA at pH 3.3 and 6.7.
i) Experimental objectives and rationale:
   Since previous studies showed a significant effect on the binding of Ptx to HSA and formulation stability at acidic pH, this study was designed to
      Evaluate the reproducibility of the effect using radioactive Pix.
      Compare HSA defatted with different types of charcoals.
      Evaluate the combined effect of pH and molar ratios on the binding.
      Evaluate the pH-dependent stability of the formulation mixtures.
ii) Experiment:
   HSA-D (defatted with different charcoal impregnated filter media) was analyzed at 2:1 and 1:1 Ptx/HSA molar ratio, in 4% ethanol.
   Ptx concentrations tested were 200 and 400 μg/mL.
   Buffer systems: pH 3.3 and 6.7 McIlvaine buffer solutions.
iii) Results and conclusion:
   Defatting of HSA with different types of charcoal media did not affect the binding of Ptx to HSA (Table 15). Consequently, the results from the different types of charcoal can be averaged out in Table 15.
   Higher recovery and binding were obtained at the acidic pH (3.3) than at the neutral pH (6.7) at both molar ratios of 2:1 and 1:1.
   Increasing the Ptx concentration from 200 to 400 μg/mL resulted in a decrease in recovery as well as in binding.

This effect can be minimized by improved mixing techniques to avoid localized high concentration of Ptx that forms precipitates (achieved with the recent formulation mixtures, see studies on effect of Ptx concentrations).

TABLE 15
Evaluation of the effects of different carbon-impregnated media
for defatting HSA, molar ratio, and Ptx concentration on the binding
of Ptx to HSA at two different pH in 4% ethanol solutions.

| | | | pH 3.3 | | pH 6.7 | |
|---|---|---|---|---|---|---|
| | Grade of | | Total | Estimated | Total | Estimated |
| Ptx excess | filter for | | soluble Ptx | HSA | soluble Ptx | HSA |
| molar | defatting | Ptx conc. | Recovery⁽²⁾ | bound Ptx | Recovery | bound |
| amount | HSA⁽¹⁾ | (μg/mL) | (%) | (%) | (%) | Ptx (%) |
| 1 | HC | 200 | 94.1 | 89.8 | 55.2 | 54.5 |
| 1 | KB | 200 | 95.1 | 92.1 | 57.2 | 54.8 |
| 1 | SX | 200 | 94.5 | 91.8 | 53.2 | 50.4 |
| 1 | CR | 200 | 96.7 | 92.6 | 55.6 | 53.4 |
| 1 | HC | 400 | 83.6 | 82.1 | 36.7 | 34.9 |
| 1 | KB | 400 | 86.2 | 84.8 | 43.2 | 40.4 |
| 1 | SX | 400 | 84.9 | 83.2 | 42.2 | 39.9 |
| 1 | CR | 400 | 87.8 | 86.1 | 38.4 | 36.8 |
| 2 | HC | 400 | 73.2 | 69.8 | 33.6 | 30.6 |
| 2 | KB | 400 | 75.3 | 72.3 | 36.0 | 32.9 |
| 2 | SX | 400 | 78.7 | 75.5 | 31.5 | 28.5 |
| 2 | CR | 400 | 79.5 | 76.4 | 34.6 | 31.7 |

⁽¹⁾Cellulo Carbac carbon impregnated media. HC: acid washed, steam activated; lignite-based carbon; KB: chemically activated, wood-based carbon; SX: acid washed, steam activated, peat-based carbon; and CR: chemically activated, pine wood carbon.
⁽²⁾Total soluble Ptx consists of HSA-bound Ptx and unbound Ptx in solution, estimated after removal of insoluble Ptx. The results are averages of triplicate data points.

Experiment 2. Determination of the Effect of pH on the Binding of Ptx to HSA-B and HSA-D at 1:1 Molar Ratio.
(i) Experimental objectives and rationale:
   This study was designed to determine the pH profile of the binding of Ptx to HSA in a broader pH range extending to alkaline pH (3.0 to 9.0) as well as the formulation stability at this pH rang. The two types of HSA (undefatted defatted) were compared at 1:1 molar ratio with Ptx to help in determining the suitable HSA preparation for product development.
ii) Experiment:
   HSA-B and HSA-D were analyzed at 1:1 Ptx/HSA molar ratio, 200 μg/mL Ptx in 4% ethanol.
   Buffer systems: different pH McIlvaine buffer solutions.
   pH tested: pH 3.0, 3.2, 3.4, 3.6, 3.8, 4.0, 4.2, 4.4, 4.6, 5.0, 5.5, 6.0, 6.5, 7.0, 7.5, 8.0, 8.5 and 9.0
      Below is the procedure for the analysis of the effect of pH on the recovery and binding of soluble Ptx to both defatted and undefatted HSA.
2. Materials
   2.1 HSA-B (12%) solution, acidic pH undefatted (pH 3.1-3.3).
   2.2 HSA-D (12%) solution, acidic pH defatted (pH 3.1-3.3).
   2.3 Paclitaxel stock solution: 5 Ptx (5 mg/mL) in dehydrated EtOH, containing tritiated Ptx at 1/100 dilution.
      Ethanol solution must be dehydrated.
   3.4 1×McIlvaine buffer solutions of different pH values, ranging from 3.0 to 9.0.
3. Reaction and analysis conditions
   4.1 Ptx concentration: 200 μg/mL, containing tritiated Ptx.
   4.2 HSA concentrations: 15.6 mg/m, for a molar ratio of 1:1.
   4.3 EtOH concentration: 4% (v/v).
   4.4 All reaction mixtures will be in triplicates.
   4.5 Samples will be incubated at 23° C. for 4 days.
   4.6 Reaction mixtures will be analyzed to quantitate the amount of Ptx in the soluble and HSA-bound form, at day 0, day 1 and day 4, to estimate the stability of the formulation with time.
4. Procedure
   5.1 Prepare 1×McIlvaine buffer solutions of different pH (12-15 mL each), according to the Table below.
   5.2 Prepare 2 HSA solutions: HSA-B and HSA-D at a concentration of 12% (w/v).
   5.3 In 2 sets of test tubes, aliquot out 2 mL of each HSA preparation (a test tube/each test reaction pH), according to the Table below.
   5.4 Adjust the pH of each HSA solution to the corresponding pH with known volume of mild NaOH or H₃PO₄.
   5.5 Adjust the concentration of each HSA solution with WFI (water for injection) to 10% (w/v).
   5.6 Set up clean test tubes in triplicate per pH and per HSA type (i.e. 18×3×2=108 total) for radioactive Ptx/HSA reaction mixtures.
   5.7 Set up clean test tubes per pH and per HSA type (i.e. 18×1×2=36 total) for non-radioactive Ptx/HSA reaction mixtures. This set will be used for analysis of turbidity at 600 nm wavelength).

5.8 Add all reagents at 23° C., starting with HSA, then buffer followed by Ptx/EtOH.

| | 2-mL reaction mixture |
|---|---|
| 10% HSA (HSA-B or HSA-D) | 312 μL |
| 1 × McIlvaine buffer solution | 1608 μL |
| 5 Ptx solution⁽¹⁾ (radioactive or non-radioactive) | 80 μL |

⁽¹⁾The 80 μL of 5 Ptx should be added slowly while vortexing.

5.9 Allow the reaction mixtures to incubate for 4 days.
5.10 Analyze the binding by LSC. Measure fractions R, S and F for day 0, day 1 and day 4.

5.11 Collect data on from a printout and on a diskette for processing and analysis by Excel.

TABLE 16
Selected reaction pH for analysis of Ptx binding
to HSA-B and HSA-D.

| | | 12% HSA-B | 12% HSA-D |
|---|---|---|---|
| Solution | Reaction pH | (mL) | (mL) |
| 1 | 3.0 | 2 | 2 |
| 2 | 3.2 | 2 | 2 |
| 3 | 3.4 | 2 | 2 |
| 4 | 3.6 | 2 | 2 |
| 5 | 3.8 | 2 | 2 |
| 6 | 4.0 | 2 | 2 |
| 7 | 4.2 | 2 | 2 |
| 8 | 4.4 | 2 | 2 |
| 9 | 4.6 | 2 | 2 |
| 10 | 5.0 | 2 | 2 |
| 11 | 5.5 | 2 | 2 |
| 12 | 6.0 | 2 | 2 |
| 13 | 6.5 | 2 | 2 |
| 14 | 7.0 | 2 | 2 |
| 15 | 7.5 | 2 | 2 |
| 16 | 8.0 | 2 | 2 |
| 17 | 8.5 | 2 | 2 |
| 18 | 9.0 | 2 | 2 |

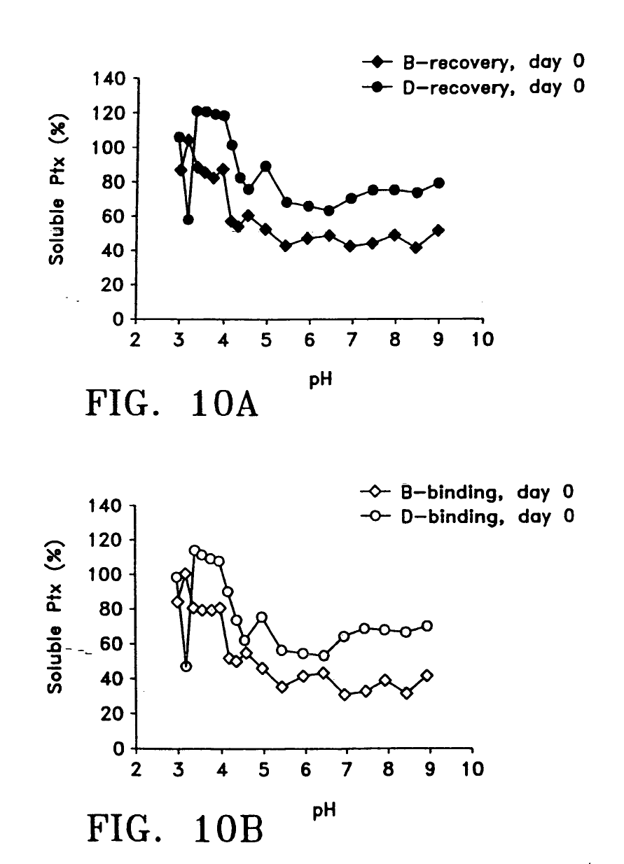

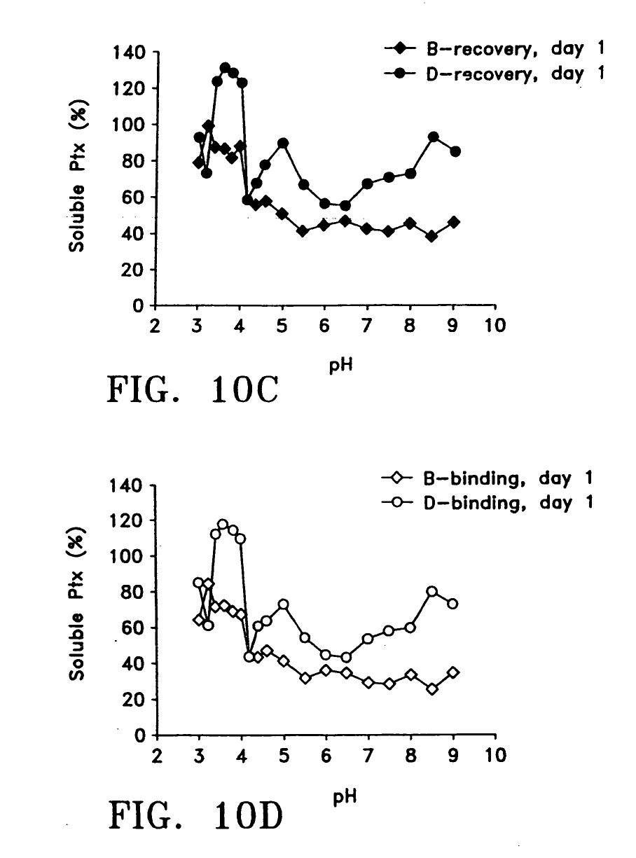

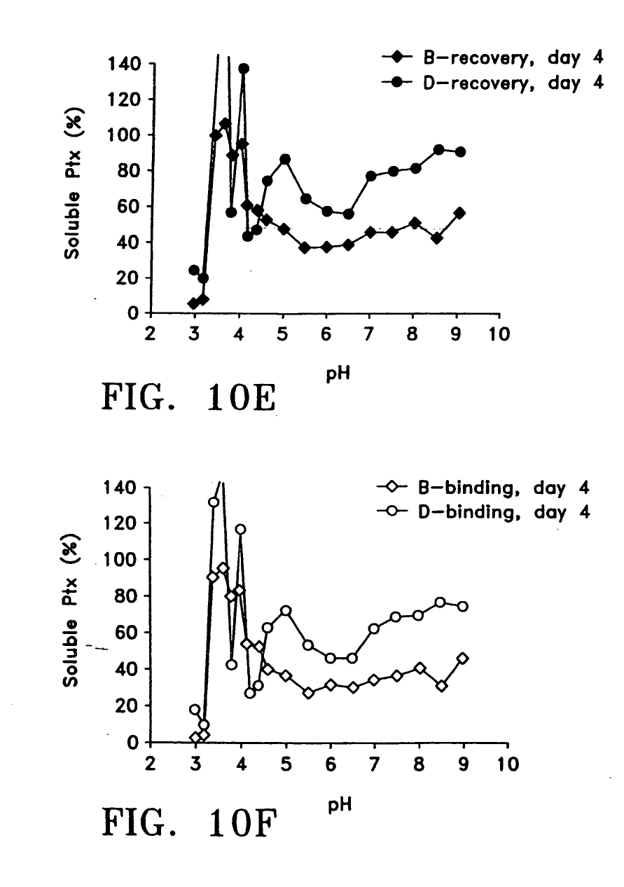

FIG. 10 depicts the pH profile for recovery and binding of Ptx to defatted and undefatted HSA and the stability of the resulting Ptx:HSA formulations.

Results and Conclusion:

Highest binding was obtained in the acidic pH range of 3.0 to 4.0, thus confirming previous studies (FIG. 10). The increased binding was not affected by the defatting of HSA. However, after 4 days of storage, the formulation stability was significantly reduced at pH 3.2 or lower (FIG. 10, Panels E and F). Increasing the pH from 4.0 to neutral, resulted in at least a two-fold decrease in the binding. The resulting formulation mixtures were unacceptable due to increased amount of Ptx precipitation. The two-fold reduction in the binding observed at near neutral pH is consistent with the hypothesis that an additional binding site is mad available at acidic pH. Since the paclitaxel available from BMS (Bristol Myers Squibb) has been formulated at pH 9.1, we extended the pH study for the first time, to evaluate the effect of binding and the formulation stability in the alkaline pH range, for both undefatted and defatted HSA. Surprisingly, the defatting of HSA had a beneficial effect on the binding of Ptx to HSA. The binding increased from 30 to 40% with undefatted HSA to 68-75% with defatted HSA. Because of known instability of Ptx at alkaline pH—the basis for the NaPro BioTherapeutics, Inc. (1998) patent as an improvement over the BMS formulation, we tested also the effect of alkaline pH for potential of reconstitution of a 24-h stable formulation. Chemical stability of Ptx in these alkaline pH formulations was found poor.

1. J. Analysis of the Effect of Molar Ratio on the Binding of Ptx to Different Types of HSA in Saline.

The following study analyzed the effect of different HSA types and molar ratios on the recovery of soluble Ptx and binding to HSA at 4% and 20% ethanol, as well as the formulation stability after 24 h of storage at room temperature. The following HSA preparations were analyzed, including: undefatted neutral and acidic pH (HSA-A and HSA-B); defatted neutral pH (HSA-C) and dialyzed undefatted neutral and acid pH (HSA-Au and HSA-Bu).

Experiment 1.
1.1 Experiment #1: 4% ethanol molar ratio study.
   Various types of HSA analyzed with at different molar ratios ranging from 1:4 to 2:1, in 4% ethanol.
   Ptx concentrations tested were 200 μg/mL.
   Recation solutions: pH 3.3 and 7.0 saline solutions.
1.2 Experiment #2: 20% ethanol molar ratio study.
   Various types of HSA analyzed with at different molar ratios ranging from 1:4 to 2:1, in 20% ethanol.
   Ptx concentrations tested were 200 μg/mL.
   Recation solutions: pH 3.3 and 7.0 saline solutions.
      Experimental procedure. The experimental procedure for the evaluation of the effect of molar ratio with different HSA types is described below.
3. Materials
   3.1 Different HSA solutions (10% w/v).
   Prepare at least 13 mL each.
      10% HSA-A, undialyzed neutral pH undefatted (pH 6.8-7.0).
      10% HSA-B, undialyzed acidic pH undefatted (pH 3.1-3.3).
      10% HSA-C, undialyzed neutral pH defatted (pH 6.8-7.0).
      10% HSA-Ad, dialyzed neutral pH undefatted (pH 6.8-7.0)
      10% HSA-Bd, dialyzed acidic pH undefatted (pH 3.1-3.3).
   3.2 Radioactive 5 Ptx solution (5 mg/mL paclitaxel in dehydrated EtOH containing hot Ptx at 1/200 dilution). Prepare at least 7 mL for the experiment (i.e. 0.08 mL×75=6 mL).
   3.3 Sterile concentrated saline solution (1.64% NaCl). Prepare 100 mL.
   3.4 Sterile acidified WFI, pH 3.3 with phosphoric acid. Prepare 100 mL.
   3.5 Sterile WFI, pH near neutral (adjusted to 6.8-7.0 with NaOH if necessary)
4. Procedure
   4.1 Preparation of 1.64% saline solution for the 4% ethanol concentration reaction mixtures.
      Adjust the pH of the saline to the pH of the reaction (i.e. pH 3.3 and 7.0). Note that for the neutral pH reaction, the saline solution needs not be adjusted.
   4.2 Preparation of HSA sub-stock solutions for the different molar ratios.
      4.2.1 With each of the four 10% HSA solutions make 5 sub-stocks of HSA solutions by mixing different amounts with WFI as per Table 2 for the corresponding molar ratios of 1:0.5, 1:1, 1:2, 1:3 and 1:4, respectively.
      4.2.2 For the acidic HSA solutions, prepare the sub-stock solutions by mixing with acidified WFI to minimize the change in pH with decreasing amount of HSA at higher molar ratios.
      4.2.3 For the neutral pH HSA solutions, prepare the sub-stock solutions by mixing HSA with near neutral pH WFI to minimize the change in pH with decreasing amount of HSA at higher molar ratios.
   4.3 Formulation reaction mixture preparation.
      4.3.1 Formulation conditions:
         4.3.1.1 Ptx concentration: 200 μg/ml.
         4.3.1.2 Molar ratio: varying from 1:0.5 to 1:4.
         4.3.1.3 HSA concentration: varying from 7.8 to 62.4 mg/mL.
         4.3.1.4 Ethanol concentration: 4% (v/v).
         4.3.1.5 Binding solution: saline (final concentration of 0.55% NaCl).
      4.3.2 For each of the five HSA types, set up 5 tubes in triplicate for the 5 molar ratios, for a total of 5×5×3=100 tubes as per Table 17.
      4.3.3 To appropriate tubes add 1250 μL of the right HSA sub-stock in triplicates.
      4.3.4 Add to each tube 670 μL of 1.64% NaCl solution to each tube.
      4.3.5 With constant mixing, slowly add 80 μL of radioactive 5 Ptx solution.
   4.4 Sample analysis.
      4.4.1 Analyze the recovery and binding by LSC.
         4.4.1.1 Day 0: R, S and F.
         4.4.1.2 Day 1: S and F.

4.4.2 Collect data for processing and analysis by excel.

TABLE 17
Ptx/HSA molar ratio study: Experimental design for the amount of HSA
required in the reaction mixtures to obtain different Ptx:HSA molar ratios.

| Reaction condition | | | | HSA solutions for different molar ratios | | |
|---|---|---|---|---|---|---|
| | | | Concentrated | | | Required |
| | | | HSA | Required | | amount |
| | Final | Final | stock | amount of | Required amount | of HSA/WFI |
| | conc. of | conc. of | solution | HSA per | of WFI per | mixture per |
| Ptx:HSA | Ptx | HSA | conc. | 2-mL | 2-mL reaction | 2-mL reaction |
| molar ratio | (μg/mL) | (mg/mL) | (mg/mL) | reaction (μL) | (μL) | (μL) |
| 1:0.5 | 200 | 7.8 | 100 | 156 | 1094 | 1250 |
| 1:1 | 200 | 15.6 | 100 | 312 | 938 | 1250 |
| 1:2 | 200 | 31.2 | 100 | 624 | 626 | 1250 |
| 1:3 | 200 | 46.8 | 100 | 936 | 314 | 1250 |
| 1:4 | 200 | 62.4 | 100 | 1248 | 2 | 1250 |

The 4 HSA stock solutions concentration is 100 mg/mL (10%, w/v).

TABLE 18
Ptx/HSA molar ratio study: Experimental design for the
preparation of stock HSA solutions for the different molar ratios.

| | | | HSA solutions for different molar ratios | | | |
|---|---|---|---|---|---|---|
| | | | Concentrated | | | Required |
| | | | HSA | Required | Required | amount of |
| Reaction condition | | | stock | amount of | amount of WFI | HSA/WFI |
| | Final conc. | Final conc. | solution | HSA per | per | mixture per |
| Ptx:HSA | of Ptx | of HSA | conc. | 8-mL reaction | 8-mL reaction | 2-mL reaction |
| molar ratio | (μg/mL) | (mg/mL) | (mg/mL) | (μL) | (μL) | (μL) |
| 1:0.5 | 200 | 7.8 | 100 | 624 | 4376 | 1250 |
| 1:1 | 200 | 15.6 | 100 | 1248 | 3752 | 1250 |
| 1:2 | 200 | 31.2 | 100 | 2496 | 2504 | 1250 |
| 1:3 | 200 | 46.8 | 100 | 3852 | 1256 | 1250 |
| 1:4 | 200 | 62.4 | 100 | 4992 | 8 | 1250 |

HSA stock solutions: 10% (w/v). Total amount of HSA required per 8-mL reactions is 13.1 mL.

TABLE 19
Ptx/HSA molar ratio study: Experimental design for the amount of HSA
sub-stock required in the reaction mixtures at different molar ratios.

| | | Amount of HSA sub-stock per reaction mixture (μL)⁽¹⁾ | | | | |
|---|---|---|---|---|---|---|
| | | Undialyzed | Undialyzed | Undialyzed | Dialyzed | Dialyzed |
| Ptx:HSA molar | HSA sub-stock | HSA-A, pH | HSA-B, pH | HSA-C, pH | HSA-A, | HSA-B, |
| ratio | name | 7.0 | 3.3 | 7.0 | pH 7.0 | pH 3.3 |
| 1:0.5 | H0.5 | 1250 | 1250 | 1250 | 1250 | 1250 |
| 1:1 | H1 | 1250 | 1250 | 1250 | 1250 | 1250 |
| 1:2 | H2 | 1250 | 1250 | 1250 | 1250 | 1250 |
| 1:3 | H3 | 1250 | 1250 | 1250 | 1250 | 1250 |
| 1:4 | H4 | 1250 | 1250 | 1250 | 1250 | 1250 |

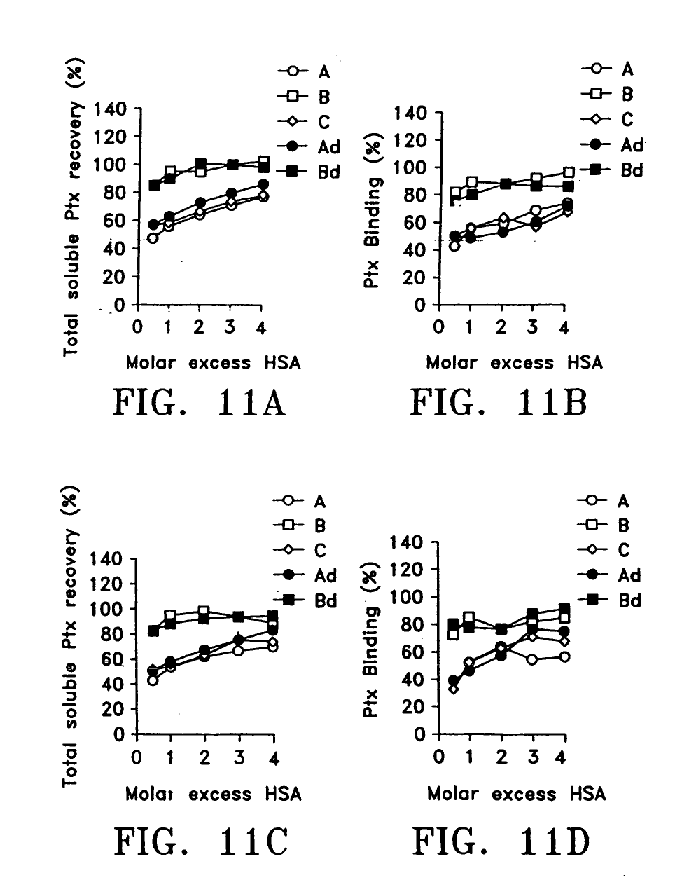

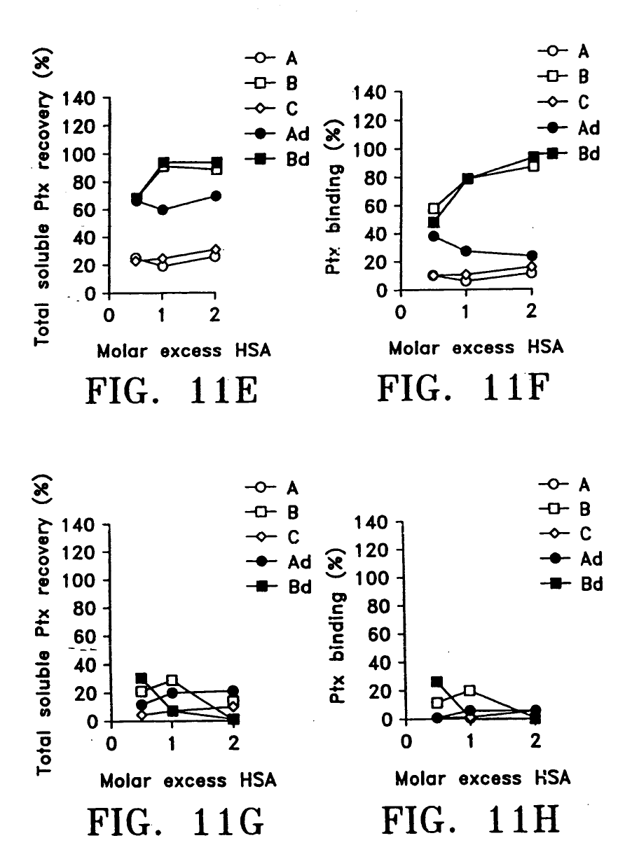

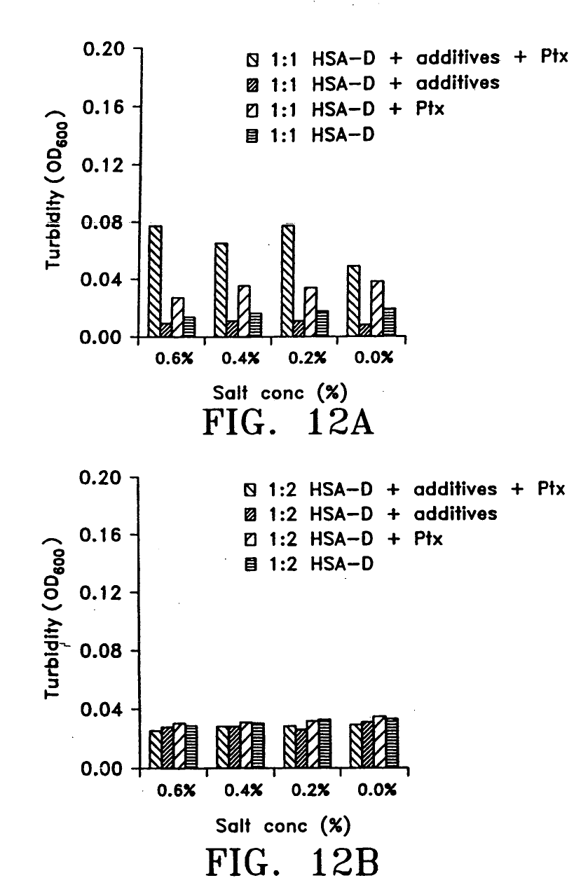

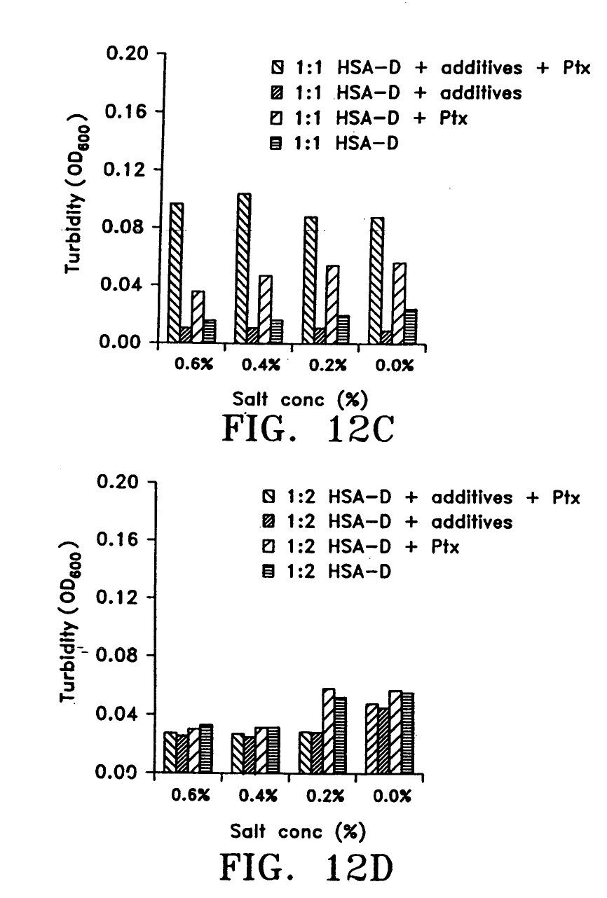

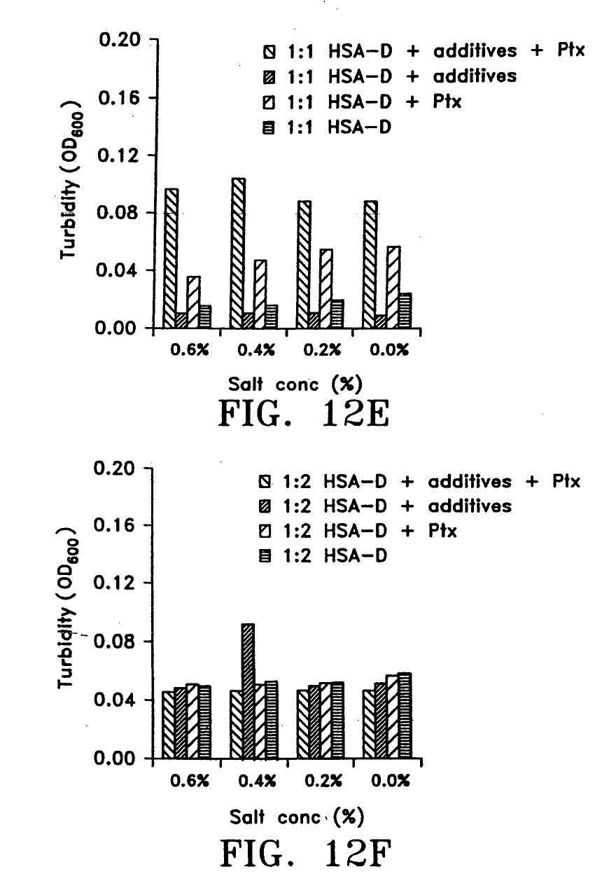

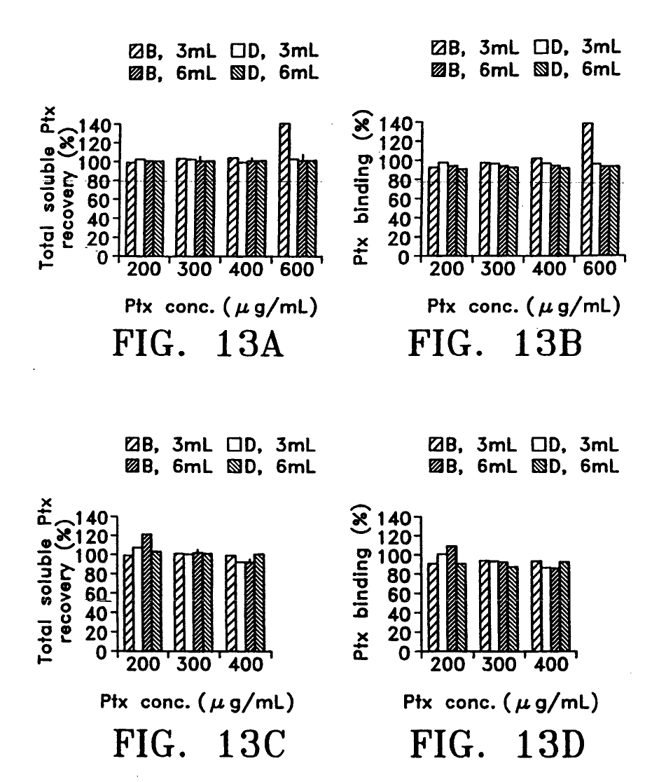

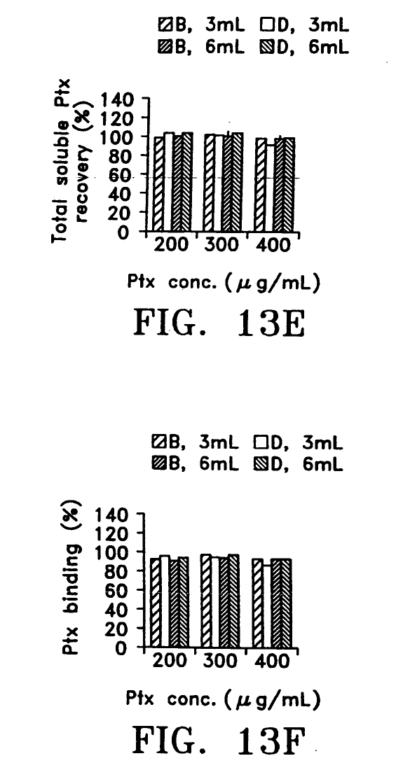

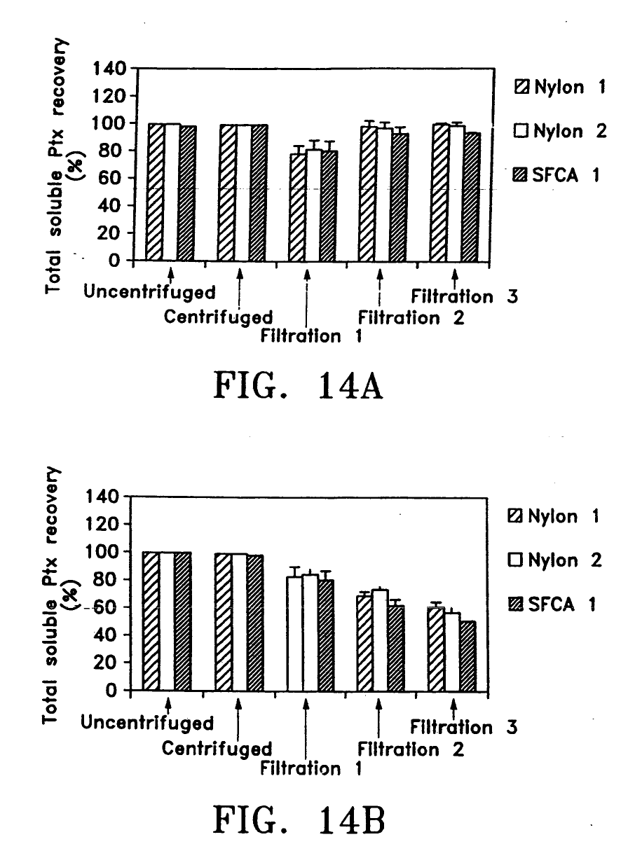

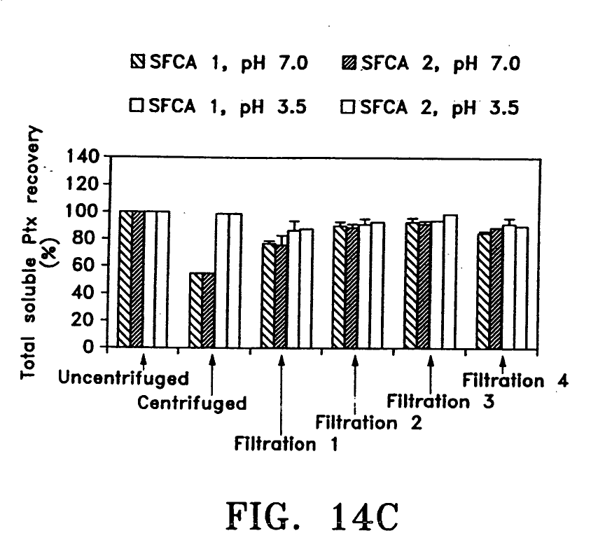

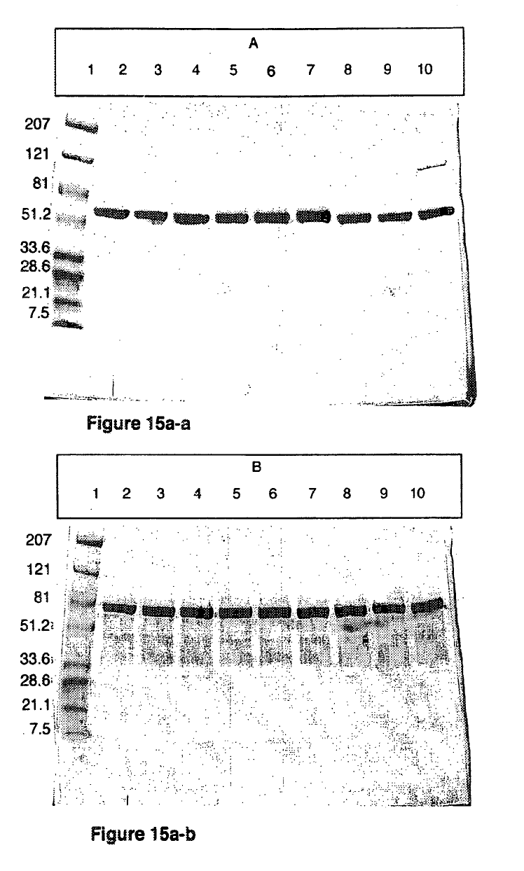

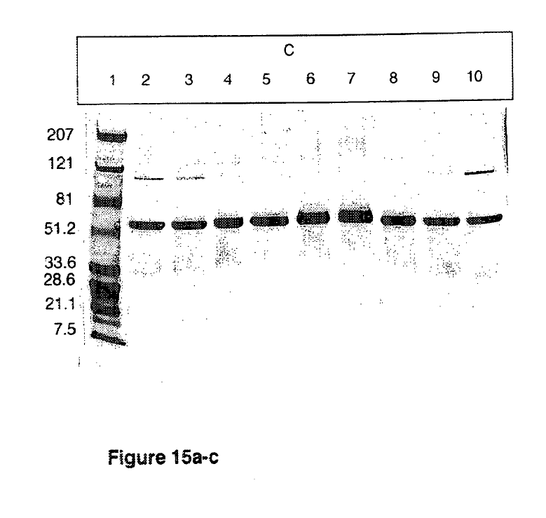

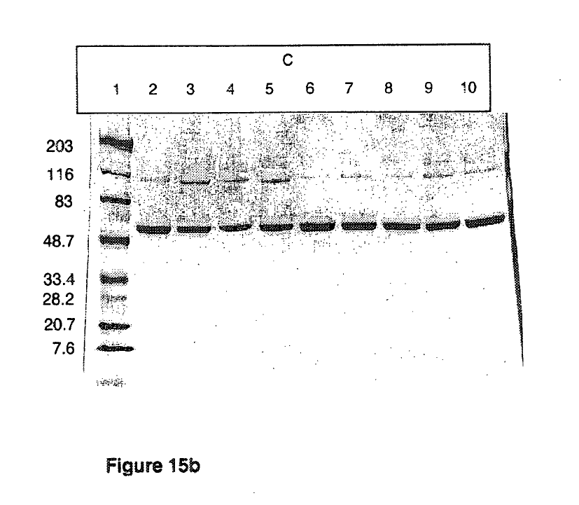

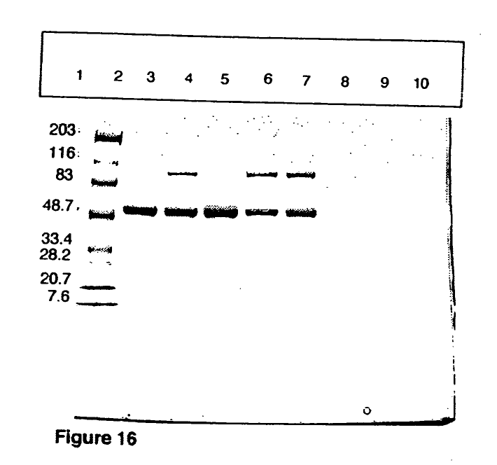

FIG. 11A depicts the effect of molar ratio on the recovery and binding of soluble Ptx HSA formulations containing 4% ethanol. Ptx, at fixed concentration of 200 μg/mL. was bound to different types of HSA (HSA-A, HSA-B. HSA-C, dialyzed HSA-A and dialyzed HSA-B) at different molar ratios in saline solution containing 4% ethanol. Quantitation of Ptx recovery was by radioactivity at day 0 (Panel A), and day 1 (Panel C); and binding at day 0 (Panel B), and day 1 (Panel D) at 23° C.

Results:

At 4% ethanol, the highest recovery and binding were obtained with acidic formulations containing undefatted undialized (HSA-B) or undefatted dialyzed (HSA-Bd) HSA types. This applied to all molar ratios tested. Increase in the recovery and binding occurred with the other types of HSA (neutral pH formulations of undialyzed and dialyzed undefatted HSA and undialyzed defatted HSA) with decreasing molar ratios from 2:1 to 1:4. Implyin obtained with acidic formulations containing undefatted undialyzed (HSA-B) or undefatted dialyzed (HSA-Bd) HSA types. The stability of all formulations was not changed significantly after 1 day at room temperature. At 20% ethanol, initial high recovery and binding were obtained with acidic formulations containing undefatted undialized (HSA-B) or undefatted dialyzed (HSA-Bd) HSA types. This applied to all molar ratios tested. However, the formulations were highly unstable after a 1-day storage at room temperature. All neutral formulations had poor recovery and binding. These data confirmed that the combination of both low pH and low ethanol is necessary to achieve high binding and stability.

FIG. 11B depicts the effect of molar ratio on the recovery and binding of soluble Ptx HSA formulations containing 20% ethanol. Ptx, at fixed concentration of 200 μg/mL, was bound to different types of HSA (HSA-A, HSA-B, HSA-C, dialyzed HSA-A and dialyzed HSA-B) at different molar ratios in saline solution containing 20% ethanol. Quantitation of Ptx recovery was by radioactivity at day 0 (Panel A), and day 1 (Panel C); and binding at day 0 (Panel B), and day 1 (Panel D) at 23° C.

TABLE 19A
Comparison of the stability of Ptx/HSA formulation under air
alone or with DTE and cysteine, and argon in different sized vials.

| | | Day 1, 40° C. | | Day 6, 23° C. | |
|---|---|---|---|---|---|
| | Vial | Total soluble | Day 1, 40° C. | Total soluble | Day 6, 23° C. |
| | size | Ptx Recovery | Estimated % | Ptx Recovery | Estimated % |
| Condition | (mL) | (%) | Bound Ptx | (%) | Bound Ptx |
| Argon | 2 | 73.1 | 66.6 | 96.4 | 85.9 |
| Argon | 20 | 81.3 | 74.0 | 95.9 | 85.9 |
| Air | 2 | 72.8 | 66.4 | 88.8 | 82.7 |
| Air | 20 | 45.8 | 41.4 | 92.3 | 84.8 |
| Air + Cys/DTE | 2 | 64.8 | 58.8 | 94.5 | 88.1 |
| Air + Cys/DTE | 20 | 60.5 | 54.7 | 98.6 | 93 |

2-mL formulations were filled in the vials and stored at indicated temperature.

1. K. Effect of Salts

Experiment: Analysis of the Effect of Salts on the Stability of Ptx/HSA Formulation.

The following study analyzed the combined effect of salt and ethanol concentrations on the stability of defatted acidic HSA (HSA-D) at Ptx to HSA molar ratio of 1:1 and 1:2. Also analyzed is the effect of additives, N-acetyl-tryptophane and caprylic acid, normally added to commercial HSA preparation as stabilizers, on the stability of the formulation.

Experiment 1.
iv) Experimental objectives and rationale:
   Since previous studies showed a significant effect of ethanol concentration on the binding and stability of the Ptx/HSA formulation stability, this study was designed to
      Evaluate the effect of different HSA preparations: undefatted acid and neutral pH, defatted neutral pH, and dialyzed undefatted neutral and acidic pH. On the binding of Ptx at different molar ratios in saline solution containing 4% ethanol. A similar study was conducted at 20% ethanol.
v) Experiment # 1: 1:1 molar ratio study.
   Defatted acidic HSA (HSA-D) was analyzed at 1:1 molar ratio of Ptx to HSA.
   Ptx concentration was fixed at 200 μg/mL.
   Reaction solutions: different concentration NaCl solutions containing 4, 6 and 8% ethanol, at pH 3.5.
vi) Experiment. #2: 1:2 molar ratio study.
   Defatted acidic HSA (HSA-D) was analyzed at 1:2 molar ratio of Ptx to HSA.
   Ptx concentration was fixed at 200 μg/mL.
   Reaction solutions: different concentration NaCl solutions containing 4, 6 and 8% ethanol, at pH 3.5.
vii) Results and conclusion:
   At 1:1 molar ratio (15.6 mg/mL HSA), the additives caprylic acid affected the binding of Ptx to HSA. This effect was reversed by the addition of more HSA (1:2 molar ratio).
   The effect of salt in the concentration range tested was not significant at low ethanol concentration. But it was found that polymerization of HSA (gel formation observed at high temperature, 37° C.) at acidic pH occurred at high salt and ethanol concentration.
   Consequently low ethanol concentration has an added benefit to the liquid formulation.

FIG. 4 shows the effect of salt and ethanol concentrations on the stability of the Ptx/HSA formulation. Ptx was bound to acidic defatted HSA (HSA-D) at two molar ratios 1:1 (FIG. 4A, C, E), and 1:2 (FIG. 4B, D, F) in NaCl solutions containing different concentrations of ethanol 4% (A, B), 6% (C, D) and 8% (E, F). Formulation stability was qualitatively monitored at day 0 by measuring the solution turbidity. Ptx concentration was 200 μg/mL; and the additives were caprylic acid and N-acetyl-tryptophane (4 mM each).

1. L. Effect of Antioxidants

1.1 Effect of Antioxidants on the Stability of the Ptx/HSA Formulation

Unlike chemical drugs, the Ptx/HSA formulation must demonstrate not only the chemical stability of the active ingredient Ptx but also the biochemical stability of the carrier excipient HSA. HSA contains a number of cysteine residues that form disulfide bridges in the native protein, as is known in the art. Under suitable conditions, intermolecular disulfide bridges involving cysteine residue 34 may occur, resulting in the dimerization of serum albumin. We found that the acidification of HSA to pH 3.0-3.3, as required for an effective removal of bound fatty acids with carbon impregnated media, as is known in the art, causes HSA to form an unacceptable number of dimers and trimers. This experiment evaluated a number of conditions to maintain HSA in the formulation, primarily in the monomeric form. Different antioxidants were analyze to determine the optimal conditions for stabilizing the Ptx/HSA formulation.

i) Experiment #1:

Evaluation of different antioxidants with undefatted and defatted acidic HSA preparations.
The following reducing reagents were tested: ascorbic acid, L-cysteine, dithioerythritol (DTE) and dithiothreitol (DTT), sodium metabisulfite, sodium thiosulfate, and thioacetic acid.
Different preparations of HSA (10%), including HSA-A, HSA-B, and HSA-D, were treated with antioxidant at different concentrations, ranging from 2 to 40 mM.
The HSA solutions were incubated at 2-8° C. for up to a month, and prevention of dimerization was analyzed by SDS-PAGE under both reducing and non-reducing conditions.

Analysis of the effect of antioxidant on prevention of HSA dimerization at low pH.
5. Materials
   2.1 HSA-A solution (20% w/v), neutral pH undefatted HSA (pH 6.8-7.0).
   2.2 Other HSA solution to be prepared from HSA-A:
      10% HSA-B, acidic pH undefatted (pH 3.1-3.3).
      10% HSA-D, acidic pH defatted (pH 3.1-3.3).
   2.3 Antioxidant:
      2.3.1 Dithioerythritol: 400 mM stock solution in WFI.
      2.3.1 Cysteine: 400 mM stock solution in WFI.
   2.4 0.85 M phosphoric acid solution
   2.5 Syringes:
      2.5.1 10-mL syringes fitted with a cut-out disk of charcoal-impregnated filter media (1 syringe per each preparation of defatted sample).
      2.5.2 10-mL syringes and 0.2 micron filter for filter-sterilization of all the samples (1 syringe/filter per each HSA preparation).
         15-mL conical centrifuge (sterile).
      2.5.3 SDS-PAGE material for analysis under reduced and non-reduced conditions.
         Sample dilution buffers
         Mini Protean gel apparatus
         Staining and destaining solutions
6. Procedure
   3.1 Prepare 7 solutions (in test tubes) consisting of 400, 200 and 100 mM cysteine and dithioerythritol (DTE), and a combination of cysteine+DTE (100 mM each) as follows:
      3.1.1 400 mM cysteine solution.
         96.96 mg of cysteine in 2 mL of WFI
      3.1.2 200 mM and 50 mM cysteine solution.
         Carry 2 serial two-fold dilutions of 400 mM cysteine using 1 mL WFI as diluent. Dilution #1 and #2 are for 200 mM and 100 mM, respectively.
      3.1.3 400 mM DTE solution.
         123.3 mg of DTE in 2 mL of WFI
      3.1.4 200 mM and 100 mM DTE solution.
         Repeat step 3.1.2 for DTE
      3.1.5 100 mM cysteine+DTE solution.
         To 600 μL of WFI add 300 μL of 200 mM cysteine 300 μL of 200 mM DTE.
   3.2 Aliquot out 1.92 mL of HSA-A (20%) in 2×9 (18) 15-mL conical tubes labeled as follows:
      HSA-B set: B1-B9
      HSA-D set: D1-D9

3.3 Add 80 μL of antioxidant solution to the 1.92 mL of HSA-A (20%) in 2×9′ (18) the 15-mL conical tubes as follows:

| | Antioxidant (80 μL) | HSA-B set | HSA-D set | Final conc |
|---|---|---|---|---|
| 1 | 100 mM Cys | B1 | D1 | 2 mM Cys |
| 2 | 200 mM Cys | B2 | D2 | 4 mM Cys |
| 3 | 400 mM Cys | B3 | D3 | 8 mM Cys |
| 4 | 100 mM DTE | B4 | D4 | 2 mM DTE |
| 5 | 200 mM DTE | B5 | D5 | 4 mM DTE |
| 6 | 400 mM DTE | B6 | D6 | 8 mM DTE |
| 7 | 100 mM Cys + DTE | B7 | D7 | 2 mM |
| | | | | Cys + DTE |
| 8 | WFI | B8 | D8 | — |
| 9 | WFI | B9 | D9 | — |

Note:
sample 8 will be the control HSA-A, untreated for set B and set D.

Sample 9 will be the control sample for either HSA-B or HSA-D, untreated.
3.4 Incubate the samples at 2-8° C. for at least 4 h.
3.5 In the meantime,
   3.5.1 Prepare material for acidification and defatting of HSA.
   3.5.2 Prepare another 2×9 (18) set of 15-mL conical centrifuge tubes (sterile). Label the two sets as in step 3.3 with the following additional information: date, 10% HSA-B (or HSA-D)+final concentration of antioxidant.
      The 4 controls are untreated HSA-A (2) and HSA-B and HSA-D.
   3.5.3 Prepare the diluents for HSA to obtain concentrations suitable for loading in the gel.
3.6 After 4-h incubation, adjust the pH of the samples 1 to 7, 9 with 0.85 M phosphoric acid for the HSA-B set and filter sterilize (0.2 micron), into a new set of labeled tubes.
3.7 For the HSA-D set, defat the samples 1 to 7, 9 once using the syringe, then repeat the filtration with a 0.2 micron filter fitted to the defatting syringe, and collect the samples into a labeled set of conical tubes.
3.8 Storage and future analysis by SDS-PAGE:
   (i) All samples will be stored at 2-8° C. for 1 month, and analyzed as follows:
      Day 0 (reduced and non-reduced conditions).
      Day 7 (reduced and non-reduced conditions).
      Day 30 (reduced and non-reduced conditions).
   (ii) Data will be analyzed from scanned gels.

ii) Experiment #2:

Evaluation of the effect of DTE and cysteine on the stability of the Ptx/HSA formulation.
Different HSA types, including HSA-A, HSA-B, HSA-C and HSA-D, were treated with antioxidants and then used in the preparation of Ptx/HSA formulations at 1:2 molar ratio, with a fixed Ptx concentration of 200 μg/mL in McIlvaine buffer solutions containing 4% (v/v) ethanol.
The Ptx/HSA formulations were analyzed for HSA dimerization before lyophilization and after lyophilization and reconstitution within 24 h of storage at 23° C.
   Analysis of the Effect of Antioxidant on Prevention of HSA Dimerization in Acidic Ptx/HSA Formulation Solutions.
5. Materials
   5.1 HSA-A solution (20% w/v), neutral pH undefatted (pH 6.8-7.0).
   5.2 Other HSA solutions to be prepared from HSA-A:
      10% HSA-A, neutral pH undefatted (pH 6.8-7.0).
      10% HSA-B, acidic pH undefatted (pH 3.1-3.3).
      10% HSA-C, neutral pH defatted (pH 6.8-7.0).
      10% HSA-D, acidic pH defatted (pH 3.1-3.3).
   5.3 5 Ptx solution (5 mg/mL paclitaxel in dehydrated EtOH).
   5.4 Sterile 1× McIlvaine buffer, pH 3.0, with mannitol (3%).
   5.5 Antioxidant solutions:
      3.1.1 Dithioerythritol: 400 mM stock solution in WFI.
      3.1.2 Cysteine: 400 mM stock solution in WFI.
   5.6 0.85 M phosphoric acid and 0.2 M NaOH solutions.
   5.7 Syringes:
      5.7.1 10-mL syringes fitted with a cut-out disk of charcoal-impregnated filter media (1 syringe per each preparation of defatted sample, total of 2).
      5.7.2 10-mL syringes and 0.2 micron filter for filter-sterilization of all the samples (1 syringe with filter/each HSA preparation).
      5.7.3 15-mL (20) and 50-mL (8) sterile conical centrifuge tubes.
      5.7.4 SDS-PAGE material for analysis under reducing and non-reducing conditions.
         Sample dilution buffers.
         Mini Protean gel apparatus.
         Staining and destaining solutions.
   5.8 24 labeled 10-mL Serum vials with rubber stoppers for lyophilization.
6. Procedure
   4.1 Prepare an antioxidant solution consisting of a mixture of cysteine and DTE (200 mM each) as follows:
      6.1.1 400 mM cysteine solution.
         96.96 mg of cysteine in 2 mL of WFI.
      6.1.2 400 mM DTE solution. 123.3 mg of DTE in 2 mL of WFI.
      6.1.3 Add 1 mL of 400 mM cysteine to 1 mL of 400 mM DTE to make a solution of 200 mM cysteine+200 mM DTE.
   6.2 Prepare two HSA solutions in 15-mL conical tubes labeled as follows:
      6.2.1 (HSA-DTE/Cys): add 11.76 mL of 20% HSA and 240 μL WFI.
      6.2.2 (HSA+DTE/Cys): add 11.76 mL of 20% HSA and 240 μL DTE+Cys solution (200 mM prepared in 4.1.3). Note the concentration of DTE and Cys in the HSA solutions would be 4 mM each.
   6.3 Incubate the two HSA solutions for at least 4 h, at 2-8° C.
   6.4 During the incubation time, do the following:
      6.4.1 Prepare the following solutions:
         1. 80 mL of 1× McIlvaine buffer solution, pH 3.0, with 3% mannitol.
         2. Phosphoric acid (0.85 M) and NaOH (0.2 M) in sufficient amounts.
         3. Diluents for HSA to obtain concentrations suitable for loading in the polyacrylamide gel.
         4. 5 mL of 5 Ptx solution (5 mg/mL), preferably within 1 h of use.
      6.4.2 Prepare material for defatting of HSA (2 syringes with charcoal filter).
      6.4.3 Determine the amount of acid required to lower the pH of 2 mL of HSA solution (20%, w/v) to 3.1-3.3 with 0.85 M phosphoric acid, to make HSA-B (acidic pH undefatted HSA, pH 3.1-3.3) from HSA-A.
      6.4.4 Determine the amount of base required to raise the pH of 2 mL of HSA-D solution (12%, w/v) to 6.8-7.0 with 0.2 M NaOH.
      6.4.5 Pre-label 2 sets of 8 15-mL conical tubes (total 16) for the preparation of different types of 10% HSA solutions [untreated (1-4) and treated (5-8) with antioxidants] as follows:
         HSA solution # Condition
            1. 10% HSA-A (−2 mM DTE/Cys).
            2. 10% HSA-B (−2 mM DTE/Cys).
            3. 10% HSA-C (−2 mM DTE/Cys).
            4. 10% HSA-D (−2 mM DTE/Cys).
            5. 10% HSA-A (+2 mM DTE/Cys).
            6. 10% HSA-B (+2 mM DTE/Cys).
            7. 10% HSA-C (+2 mM DTE/Cys).
            8. 10% HSA-D (+2 mM DTE/Cys).
         Note that the first set of 8 tubes is used in the preparation of HSA solutions before filter-sterilization. The second is for sterile solutions after filtration through 0.2 micron filter fitted to 10-mL syringe.

6.4.6 Pre-label 8 50-mL conical tubes for the preparation of formulation mixtures as follows:

| Solution # | Condition |
|---|---|
| 1. | Ptx/HSA-A (−DTE/Cys). |
| 2. | Ptx/HSA-B (−DTE/Cys). |
| 3. | Ptx/HSA-C (−DTE/Cys). |
| 4. | Ptx/HSA-D (−DTE/Cys). |
| 5. | Ptx/HSA-A (+DTE/Cys). |
| 6. | Ptx/HSA-B (+DTE/Cys). |
| 7. | Ptx/HSA-C (+DTE/Cys). |
| 8. | Ptx/HSA-D (+DTE/Cys). |

*To each tube, add 7.776 mL of 1 × McIlvaine buffer, pH 3.0, with mannitol (3%) and keep at room temperature for later addition of 3.744 mL of HSA (10%) and 0.48 mL of 5 Ptx as per Table 20 below in step 4.7.

6.5 After the 4-h incubation of HSA solutions from step 4.2, do the following:
   6.5.1 Remove 2.5 mL from HSA-DTE/Cys solution (untreated with antioxidant, step 4.2.1) into a 15-mL conical tube, pre-labelled for HSA-A (#1, from step 4.4.5 above). Dilute this solution two-fold with WFI to make a 10% HSA-A solution.
   6.5.2 To the remaining 9.5 mL of HSA-DTE/Cys solution, adjust the pH with 0.85 M H₃PO₄ to 3.1-3.3 based on the information from step 4.4.3, and dilute the HSA solution to 12% with WFI (i.e. the final volume should be 15.8 mL). This is the 12% HSA-B solution.
   6.5.3 Remove 5 mL of the 12% HSA-B solution in the pre-labelled tube #2 from step 4.4.5. Dilute this 12% HSA-B solution with 1 mL of WFI to make a 10% HSA-B solution.
   6.5.4 Defat the remaining 10.8 mL of 12% HSA-B by passing it through a charcoal filter using a 10-mL syringe, twice (reusing the same charcoal filter). Collect the filtrate in a clean 15-mL conical centrifuge tube. This is a 12% HSA-D solution.
   6.5.5 Remove 5 mL of the 12% HSA-D solution in the pre-labelled tube #4 from step 4.4.5. Dilute this 12% HSA-D solution with 1 mL of WFI to make a 10% HSA-D solution.
   6.5.6 Remove another 5 mL of the 12% HSA-D solution in the pre-labelled tube #3 from step 4.4.5. Adjust the pH to 6.8-7.0 with 0.2 M NaOH, based on the information from step 4.4.4. And bring the volume of the HSA solution to 6 mL with WFI to make a 10% HSA-C solution.
   6.5.7 Repeat steps 4.5.1 to 4.5.6 with the second solution of HSA+DTE/Cys (treated with antioxidant, step 4.2.2).
6.6 Once all 8 10% HSA solutions are prepared, filter-sterilize them, using a 0.2 micron filter. The filtered solutions are collected in the second set of pre-labelled tubes from step 4.4.5.
6.7 Formulation preparation.
   6.7.1 Prepare 8 formulation mixtures in the pre-labelled 8 50 mL conical centrifuge tubes from step 4.4.6, as per Table 20 below.
   6.7.2 Note that the addition of buffer solution (7.776 mL) was already done in step 4.4.6.
   6.7.3 Add 3.744 mL of HSA solution to the corresponding formulation mixture. Tube (i.e. HSA solution #1 to formulation solution # 1, and so on).
   6.7.4 Add 480 μL of 5 Ptx to each test tube slowly while vortexing.
   6.7.5 Centrifuge the 8 formulation mixtures for 20 min, at 3400 rpm in the IEC centrifuge.
6.8 Formulation Lyophilization.
   6.8.1 After centrifugation, remove 1 mL from each formulation mixture for analysis by SDS-PAGE under reducing and non-reducing conditions as per step 3.9. The HSA concentration in these formulation mixtures is 3.12% (31.2 mg/mL).
   6.8.2 Incubate the unused portion of the 1-mL samples at 23° C. and reanalyze them by SDS-PAGE after day 7.
   6.8.3 With the remaining approximately 10 mL of each of the 8 formulations (200 μg/mL Ptx, 1:2 molar ratio, 4% EtOH, 1.9% mannitol) aliquot out 3 mL in pre-labelled 10-mL serum vials, in triplicate.

6.8.4 Sent the 24 serum vials (3×8) to Ted for lyophilization

TABLE 20
Preparation of different HSA formulation mixtures of Ptx/HSA (200 μg/mL
Ptx, 1:2 molar ratio, 4% EtOH) with and without antioxidants cysteine and DTE.

| | | | 1× | | |
|---|---|---|---|---|---|
| | | | McIlvaine, | | |
| | | | pH 3.0, with | | |
| | | | 3% mannitol | 10% HSA | 5Ptx |
| Solution # | Sample name | Condition | (mL) | (mL) | (mL) |
| 1 | PH23-4.200: 2A/3.0-51 | Ptx/HSA-A (−DTE/Cys) | 7.776 | 3.744 | 0.48 |
| 2 | PH23-4.200: 2B/3.0-51 | Ptx/HSA-B (−DTE/Cys) | 7.776 | 3.744 | 0.48 |
| 3 | PH23-4.200: 2C/3.0-51 | Ptx/HSA-C (−DTE/Cys) | 7.776 | 3.744 | 0.48 |
| 4 | PH23-4.200: 2D/3.0-51 | Ptx/HSA-D (−DTE/Cys) | 7.776 | 3.744 | 0.48 |
| 5 | PH23-4.200: 2A/3.0-51 | Ptx/HSA-A (+DTE/Cys) | 7.776 | 3.744 | 0.48 |
| 6 | PH23-4.200: 2B/3.0-51 | Ptx/HSA-B (+DTE/Cys) | 7.776 | 3.744 | 0.48 |
| 7 | PH23-4.200: 2C/3.0-51 | Ptx/HSA-C (+DTE/Cys) | 7.776 | 3.744 | 0.48 |
| 8 | PH23-4.200: 2D/3.0-51 | Ptx/HSA-D (+DTE/Cys) | 7.776 | 3.744 | 0.48 |

6.9 SDS-PAGE analysis of the 8 liquid formulations before lyophilization.
   6.9.1 Dilute each of the Ptx/HSA formulation mixtures to a suitable concentration of HSA for SDS-PAGE analysis under reducing and non-reducing conditions.
   6.9.2 The samples in the loading buffer can be stored in the fridge, and the gels run the following day.
   6.9.3 The scanned gels will be analyzed for the effect of antioxidant on the prevention of HSA dimerization in the formulation mixtures.
6.10 SDS-PAGE analysis of the lyophilized formulations after reconstitution.
   6.10.1 Reconstitute a set of each formulation mixture (8 vials) with 3 mL WFI,
   6.10.2 Analyze the samples by SDS-PAGE at day 0, day 1 and day 7 after reconstitution during storage at 23° C.
   6.10.3 For each analysis, dilute each of the reconstituted Ptx/HSA formulation mixtures to a suitable concentration of HSA for analysis by SDS-PAGE under reducing and non-reducing conditions.
   6.10.4 The scanned gels will be analyzed for the effect of antioxidant on the prevention of HSA dimerization in the reconstituted formulation mixtures over a one-week storage period at 23° C.

iii) Results and Conclusion:
Acidification of commercial HSA from pH 7 to 3.0-3.3 resulted in the dimerization of HSA.
Evaluation of ascorbic acid, L-cysteine, dithioerythritol (DTE) and sodium thiosulfate in minimizing the dimerization reaction showed DTE to be the most effective, followed by cysteine.
DTE was required at low concentration and less pre-incubation time with HSA was required before acidification to achieve the effect.
The effect of L-cysteine was required at high concentration, and required an overnight pre-incubation with HSA before the acidification to achieve the effectiveness of DTE.
Prolonged storage also showed DTE to be more effective in maintaining HSA I the monomeric form than cysteine.
A mixture of both reagents were selected for addition to the 10% HSA solution at concentration of 2 mM each, as required stabilizing excipients in the Ptx/HSA formulation.
   1. M. Stability Under Argon

Effect of filling under non-oxidizing conditions on the stability of the Ptx/HSA formulation.

Attempts to carry out accelerated stability studies of the Ptx/HSA formulation at 40° C. were unsuccessful in yielding stable product with appreciable recovery of soluble Ptx after a 1-day incubation. Possible reasons for the instability of the formulation were: (i) the loss of ethanol in solution through evaporation, (ii) the instability of either HSA or Ptx or both under the formulation conditions, including the acidic pH, the presence of ethanol and the presence of air. The head space air in the vial may have a destabilizing effect on the Ptx/HSA complex, which could result in a limited storage stability of the formulation. This experiment analyzed the effect of non oxidizing conditions such as the formulation filling under the inert gas argon and the addition of the antioxidant mixture of DTE/cysteine on the storage stability of the formulation at different temperatures. Also analyzed was the effect of different head space volume on the stability. The formulation consisting of HSA added to Ptx at a 1:1 molar ratio was selected for in this study for it enabled a quick detection of the effect of the various parameters under evaluation.

Experiment:

Evaluation of the effect of argon on the stability of the Ptx/HSA formulation.
The formulation conditions were:
Ptx to HSA-B molar ratio of 1:1.
Ptx concentration: 200 μg/ml.
Ethanol concentration: 4% (v/v).
Buffer system: McIlvaine buffer pH 3.4.
The Ptx/HSA formulations were analyzed for soluble Ptx recovery and binding to HSA over a 1-month incubation at both 23° C. and 40° C.
5. Materials
   5.1. 2 and 20 mL serum vials
   5.2. Argon tank
   5.3. 10%1/HSA-B
   5.4. McIlvaine buffer pH 3.4
   5.5. Hot paclitaxel 5 mg/mL in ethanol (5 Ptx)
   5.6. 400 mM cysteine in water (96.96 mg/2 mL)

1.1. 5.7. 400 mM DTE in water (123.3 mg/2 mL)
2. Samples
   2.1. 150 mL paclitaxel formulation:
      23.4 mL 10% HSA-B+120 mL McIlvaine buffer pH 3.4+6 mL 5 Ptx
   2.2. 50 mL formulation with DTE and Cysteine mixture 0.3 mM each:
      50 mL from 150 mL+78 μL mixture of DTE and Cysteine at 200 mM each (1:1 of 400 mM cysteine:DTE)

2.3. 2 mL aliquots of the paclitaxel formulation at 3 replicates for each conditions pipette into 2 and 4 mL serum vial. The vials designated for argon fill up with argon gas. Close vials with stoppers and aluminum seals.

| | 2 mL vials | 20 mL vials |
|---|---|---|
| Argon | 2 mL aliquot of 6.1 | 2 mL aliquot of 6.1 |
| Air | 2 mL aliquot of 6.1 | 2 mL aliquot of 6.1 |
| Air + Cys/DTE | 2 mL aliquot of 6.2 | 2 mL aliquot of 6.2 |

2.4. Prepare 3 sets of samples as in the table.
2.5. Store one set of the samples in the incubator at 40° C. and the rest at room temperature (23° C.).
3. Measurements of the radioactivity of samples:
   3.1. R, S, F on day zero: samples from bulk reaction mixture 6.1 and 6.2.
   3.2. S,F on day one: samples stored at 40° C.
   3.3. S,F on day 6 and 24: samples stored at room temperature
      1. N. Additional Studies
      Paclitaxel and HSA Binding: Effect of Vehicle Ionic Strength

The effect of ionic strength on the binding of paclitaxel to HSA can be determined in phosphate vehicled saline solution adjusted to the optimal pH for paclitaxel binding to HSA as determined experimentally. The ionic strength will be varied by changing the concentration of NaCl as follows: 1×, 2× and 4× the normal saline solution salt concentration. This study will also evaluate the combined effect of pH and ionic strength.

Low ionic strength parenterals are preferable for patients who may require reduced intake of potassium and sodium ions.

FIGS. 6 and 8 illustrate the effect of salt concentration (NaCl) on the binding of paclitaxel to HSA and on the appearance of the solutions as analyzed spectrophotometrically at 600 nm. FIG. 6 shows the binding estimated by ELISA. One would expect the best conditions to achieve a recovery of paclitaxel in the soluble form at a concentration of approximately 200 μg/ml, and from FIG. 8, a solution with a turbidity of less than 0.1 OD₆₀₀ unit. The highly turbid solutions at 4× the saline strength formed a thick precipitate immediately, due to salting out of HSA, and also is indicative of the instability of the formulation under these conditions.

Paclitaxel and HSA Binding: Effect of Incubation Time During Stirring

The effect of incubation time on the binding of paclitaxel to HSA can be determined in the vehicles described above. Paclitaxel and HSA can be used in amounts that give soluble mixtures but not necessarily optimal binding. In this way, improvement in the binding of paclitaxel to HSA could be investigated. The reaction mixtures can be stirred in small conical flasks using a Fisher Scientific magnetic stirrer, at maximum setting. The incubation can be carried out at room temperature for 24 h. Samples were removed at 0, 3, 6, 12 and 24 h for analysis.

Paclitaxel and HSA Binding: Effect of Temperature on the Storage Stability of Paclitaxel-HSA Complex.

The temperature stability of paclitaxel-HSA complex in the optimal saline solution established above were monitored over different periods of time at 4° C., room temperature or 23° C., and 37° C. The sample mixtures were stored in small conical flasks without stirring, and small aliquots were removed at 0, 15, 30, 60 and 90 days for analysis.

Paclitaxel and HSA binding: Effect of ethanol removal by evaporation under vacuum.

The effect of removal of ethanol from solution under vacuum was also determined in the optimal saline solution established above. It is hypothesized that, as ethanol is removed from the solution, paclitaxel will either come out of solution as a precipitate or bind to HSA and remain in solution.

Paclitaxel and HSA Binding: Effect of Order of Addition of Paclitaxel to the Reaction Mixture.

The effect of the order of addition of paclitaxel to the reaction mixture was determined in the optimal saline solution established above, and with the optimal molar ratio of paclitaxel/HSA determined in earlier studies. It is hypothesized that adding paclitaxel and ethanol to a solution of HSA dropwise or slowly with a pump with mixing may result in better yield of paclitaxel/HSA complex than when HSA is added to a solution of paclitaxel and ethanol. This experiment was carried out in a small to a slightly large scale over a 12 to 24 h-period, and at an appropriate incubation temperature. Different addition rates may also be evaluated.

Paclitaxel and HSA Binding: Effect of Reconstitution Vehicles Following Freeze-Drying.

The effect of reconstitution vehicles of different ionic strength and/or pH was determined, if the optimal saline solution established above, does not completely redissolve the freeze-dried paclitaxel-HSA complex.

Paclitaxel and HSA Binding: Effect of Shorter Incubation Times Before Lyophilization

The effect of shorter incubation times before lyophilization on binding of paclitaxel to HSA will be determined.

Analysis and Test Methods

Paclitaxel Binding to HSA: Analysis of Paclitaxel Binding

To analyze the amount of paclitaxel bound to HSA in the experiments described, the bound and unbound paclitaxel were fractionated by ultrafiltration using an Amicon filtration device fitted with a 10-kDa cut-off membrane. The unbound paclitaxel in the filtrate was quantitated as described below.

Paclitaxel Binding to HSA: Quantitation of Paclitaxel Binding

Paclitaxel bound to HSA under the different experimental conditions can be evaluated by the difference method based on the fraction of unbound paclitaxel remaining in solution. This unbound fraction can be quantitated by reverse phase HPLC, and/or ELISA.

Data Evaluation

The data from each experimental condition can be analyzed statistically. The number of replicates for each sample was at least three, unless stated otherwise. Mean, variance and standard deviation can be calculated. The reported data will have three significant figures, and will include (i) the arithmetic mean, (ii) the relative measure of precision in percent, and (iii) the associated 95% level of confidence.

### EXAMPLE 2A

1.0 Objective of the Study:

Establish the In Vitro Cytotoxicity of Paclitaxel-HSA Conjugates on Human Tumor Cell Lines.

2.0 Materials and Methods.

Test and control reagents: BMS Taxol (6 mg/mL), buffer containing drug vehicles (Cremophor EL® and ethanol at 1:1 ratio), Paclitaxel-Human serum albumin (HSA) conjugates of pH 7.0 and pH 3.0, buffer containing HSA were obtained from Dr. Ange Kadima of Fermentation Dept. The PTX-HSA formulation was in lyophilized form and it was reconstituted with distilled water just before the testing of the activity. The concentration of PTX in the reconstituted material was 0.2 mg/mL.

As described in the study protocol, three human tumor cell lines were used to determine the cytotoxic activity of BMS-taxol and paclitaxel conjugates. The human colorectal adenocarcinoma (HT-29), the human epithelial adenocarcinoma of vulva (A431) and human ovarian carcinoma (SKOV-3) cell lines were obtained from ATCC.

All cell lines were grown in cultures in RPMI1640 medium containing 10% FCS at −37 C in CO₂ incubator. Tumor cells were harvested, following SOP #2.1.32 and the viability of tumor cells were determined by trypan blue dye exclusion, according to the SOP# 2.1.9. The viability of the actively growing tumor cells was tested before the initiation of the study and it was between 92-95%. Three thousand tumor cells were seeded in each well of 96 well flat bottom plates and incubated for 16 hours for the attachment of tumor cells. The old culture medium was then replaced with fresh medium containing various dilutions (10,000 nM to 0.01 nM) of BMS taxol or paclitaxel HSA conjugates or drugs vehicles (buffers) in six replicates. For positive control of cell proliferation, cells were incubated with culture medium only. Plates were incubated with drugs or buffers for various time points: (5 hours, 20 hours, 48 hours with drug followed by incubation for another 48 hours with culture medium without drug and 96 hours).

After incubation time, the number of viable cells were determined by MTS assay (Promega Cat # G 5421), as described in study protocol. MTS assay is a colorimetric assay for determining the number of viable cells present MTS (Owen's reagent) is bio-reduced to formazan by dehydrogenase enzymes of live cells. The 50% inhibitory drug concentration (IC50) value was determined as the concentration of drug that causes 50% reduction in absorbance in comparison to untreated controls (100%). All experiments were repeated at least three times.

3.0 Results:

It has been established in clinical studies that paclitaxel is very effective in the treatment of ovarian cancer². Therefore, we used a human ovarian cancer cell line, SKOV-3, as a model to determine whether paclitaxel-HSA conjugates could cause inhibition of cell proliferation and compared the cytotoxic effect with BMS-taxol. In order to determine the effect on other human tumor cell lines, we used two other human tumor cell lines (HT-29 and A431). It has been reported in literature that A-431 (human epithelial adenocarcinoma of vulva) cells are very sensitive to taxol in comparison with other cell line HT-29 (human colon adenocarcinoma)³. Therefore, we have used these two cell lines as control.

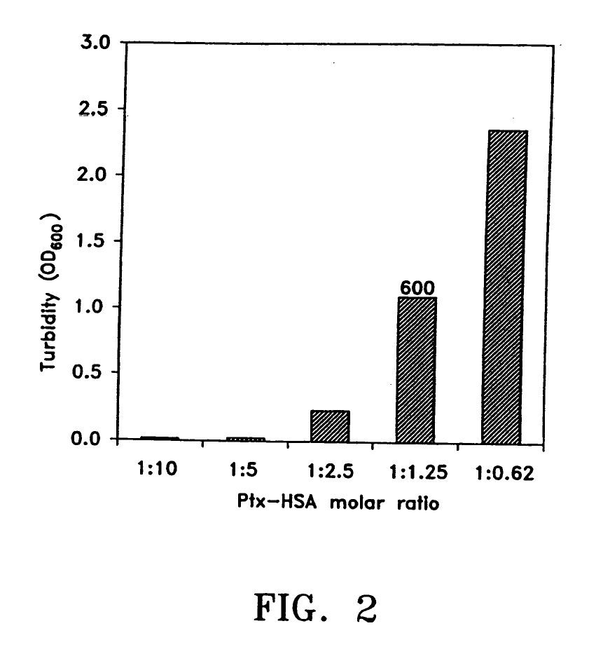

The cytotoxic activity of paclitaxel-HSA conjugates were evaluated in these three cell lines and compared with that of BMS-taxol. In the initial experiment, the cytotoxic activity of taxol and buffer containing Cremophor EL® and ethanol was tested. It was established that at higher concentration (10,000 nM), the formulation buffer containing Cremophor EL® and ethanol was cytotoxic to these human tumor cell lines, but no cytotoxicity was observed with 1000 nM or lower concentrations. The cytotoxicity was between 17%-34%, depending on the tumor cell lines tested. In contrast, the BMS taxol was cytotoxic to these tumor cells at 1-10 nM concentration. The IC50 of these cell lines ranged between 2.2 nM and 5.7 nM for BMS-taxol after exposure to 48-96 hours, as shown in Table-2. The degree of cytotoxicity was very similar, when these tumor cells were exposed to taxol for either 48 hours or 96 hours, as shown in FIG. 2. Two cell lines (A-431 and SKOV-3) showed slightly enhanced cell survival when the concentration of taxol was 10,000 nM. This observation is similar to that observed by others⁴. Therefore, in all subsequent experiments this 10,000 nM concentration was omitted.

Once the cytotoxicity of BMS taxol was established, the cytotoxicity of paclitaxel-HSA formulations of pH 7.0 and pH 3.0 was tested, using same test methodology. Unlike the buffer of BMS-taxol, the buffer containing HSA did not show any cytotoxicity to these tumor cells, the rate of cell proliferation was same with that of positive control. It was observed that like BMS-taxol, the PTX-HSA formulations (pH 7.0 & pH 3.0) were cytotoxic to these human tumor cell lines at 1-10 nM concentration. The IC50 of these cell lines ranged between 2.8 nM and 8.9 nM for PTX-HSA formulation, as against 2.2 nM and 5.7 nM for BMS-taxol after exposure to 48-96 hours; the results are shown in Table-2 and FIG. 3. Furthermore, the cytotoxicity of BMS-taxol or the PTX-HSA formulations (pH 7.0 & pH 3.0) was not increased even after exposure with the drug beyond the dose of 10 nM.

Therefore, two paclitaxel-HSA formulations were found to be very active on these human tumor cell lines in exerting the cytotoxic activity. Furthermore, the epithelial adenocarcinoma cell line A431 was found to be the most sensitive cell line for the cytotoxic effect of taxol or paclitaxel than two other cell lines tested. A dose-response curve was generated with 48 h and 96 h exposure of taxol or paclitaxel-HSA formulations and it was demonstrated that very low cytotoxic effect was observed with the increased concentrations. Therefore, studies were carried out to determine the exposure time required for the cytotoxic effect of taxol and paclitaxel HSA formulations. Cells were incubated in various concentrations of taxol or paclitaxel HSA formulations at 0.01 nM to 1000 nM concentrations for periods ranging from 5 h to 96 hours. Tumor cells suffered little or no cytotoxicity when exposed to taxol or paclitaxel-HSA formulations for only 5 hours and greatly reduced cytotoxicity after 20 hours compared to 48 hours of drug treatment, as shown in FIG. 4. However, the cytotoxic effect of BMS-taxol appeared to be slightly higher than paclitaxel-HSA formulations, when tumor cell lines were exposed to the drug for 20 hours. Therefore, it was concluded from this study that the exposure time of drug (taxol or paclitaxel HSA formulations) with the tumor cell lines is very critical in inducing the cytotoxic effect.

4.0 Conclusions:

1. Both BMS-taxol and paclitaxel-HSA formulations of pH 7.0 & pH 3.0 exerted similar cytotoxic effect on three human tumor cell lines (A431, HT-29 & SKOV-3) in a dose dependent manner up to the concentration of 10 nM.
2. This cytotoxic effect is dependent on the exposure time; the highest cytotoxic effect has been observed at 48-96 hours of exposure and lowest cytotoxic effect at 20 hours.
3. At the highest taxol concentration (10,000 nM) tested, two cell lines (A431 & SKOV-3) showed slight increase in cell survival.
4. The IC50 concentration of BMS-taxol (2.2-5.7 nM) is found to be similar with that of paclitaxel-HSA formulations pH 3.0 (2.8-8.8 nM) and pH 7.0 (3.4-8.9 nM).
   5.0 References:
1. Barltrop, J. A. et al (1991). Biorg. & Med. Chem. Lett. 1:611
2. Ling, Y. H. et al (1998). Cancer Res. 58: 3633.
3. Dosio, F. et al (1997). J. Controlled Release. 47: 293.

4. Liebmann, J. A. et al. (1993). Br. J. Cancer 68: 1104.

Determination of in vitro Cytotoxicity (IC50) of Ptx-HSA and
BMS-TAXOL to Human Tumor Cell lines

| Human Tumor | | y- | | | |
|---|---|---|---|---|---|
| Cell Lines | Sample | intercept | Slope | r² | IC50 (nM) |
| A-431 | BMS | 5.92 | −1.63 | 0.965 | 3.66 |
| (Epidermoid | Taxol | | | | |
| carcinoma) | Ptx-HSA | 6.13 | −2.03 | 0.976 | 3.61 |
| | (1-6) | | | | |
| | Ptx-HSA | 5.98 | −1.87 | 0.984 | 3.34 |
| | (2-6) | | | | |
| | Ptx-HSA | 6.30 | −2.14 | 0.984 | 4.05 |
| | (3-6) | | | | |
| | Ptx-HSA | 6.40 | −2.28 | 0.982 | 4.11 |
| | (4-6) | | | | |
| SKOV-3 | BMS | 6.46 | −1.34 | 0.977 | 12.3 |
| (Ovarian | Taxol | | | | |
| carcinoma) | Ptx-HSA | 6.43 | −1.27 | 0.957 | 13.4 |
| | (1-6) | | | | |
| | Ptx-HSA | 6.36 | −1.30 | 0.986 | 11.1 |
| | (2-6) | | | | |
| | Ptx-HSA | 6.52 | −1.38 | 0.999 | 12.6 |
| | (3-6) | | | | |
| | Ptx-HSA | 6.63 | −1.53 | 0.992 | 11.6 |
| | (4-6) | | | | |
| HT-29 | BMS | 6.0 | −1.80 | 0.982 | 3.59 |
| (Colon | Taxol | | | | |
| carcinoma) | Ptx-HSA | 7.11 | −2.95 | 0.953 | 5.19 |
| | (1-6) | | | | |
| | Ptx-HSA | 6.56 | −2.41 | 0.971 | 4.44 |
| | (2-6) | | | | |
| | Ptx-HSA | 6.82 | −2.53 | 0.973 | 5.24 |
| | (3-6) | | | | |
| | Ptx-HSA | 6.74 | −2.52 | 0.966 | 4.90 |
| | (4-6) | | | | |

TABLE 21
Establishment of the cytotoxic effect of BMS taxol and buffer containing
Cremophor-EL ® on human tumor cell lines
Percent cytotoxicity caused by the exposure of drug or drug vehicle to
human tumor cell lines for 96 hours

| | A-431* | | HT-29** | | SKOV-3* | |
|---|---|---|---|---|---|---|
| Drug | Buffer # | | Buffer # | | Buffer # | |
| conc. | (Drug | BMS- | (Drug | BMS- | (Drug | BMS- |
| (nM) | vehicle) | Taxol | vehicle) | Taxol | vehicle) | Taxol |
| 10,000 | 28 | 86 | 34 | 95 | 17 | 86 |
| 1,000 | −1 | 81 | 0 | 98 | 2 | 89 |
| 100 | 15 | 80 | −1 | 97 | 6 | 86 |
| 10 | 8 | 78 | −1 | 91 | 5 | 73 |
| 1 | 9 | 29 | ND | 18 | 5 | 9 |
| 0.1 | 6 | 14 | ND | 3 | 8 | 4 |
| 0.01 | 10 | 14 | ND | 5 | 1 | 7 |

# Drug vehicle = Buffer containing Cremophor EL ® + ethanol at 1:1 ratio.
*A-431 = Carcinoma of vulva
**HT-29 = Colon adenocarcinoma
***SKOV-3 = Ovarian carcinoma

### EXAMPLE 2B

Animal Test for Efficacy and Toxicity of Paclitaxel Formulations

Briefly, the efficacy and toxicity of paclitaxel formulations described herein can be readily tested in laboratory animals, using known methods of testing. In one such test, nude mice are injected with a xenograft of cancer cells. After tumors have developed, the mice are then injected with paclitaxel in various formulations and controls. Later the animals are checked for efficacy of treatment and side effects.

More specifically, groups of 6-8 week-old female athymic nude mice are each injected with xenografts (for example, 4 mm 3 tumor fragments or about 10⁵ to about 10⁸ cells) of breast or ovarian cancer cells. After tumors have developed (5 days after implant), the mice are assessed and distributed into groups of homogenous tumor size and shape. On day 7, 14, 21, or 28 after implant, depending on the cell line used, mice are injected with paclitaxel. The formulations of paclitaxel tested can include:
(a) Paclitaxel in serum albumin;
(b) Paclitaxel in Cremophor,
(c) Paclitaxel in Cremophor and serum albumin; and
(d) Control samples containing all components except paclitaxel.

Paclitaxel formulation (a) is prepared as described in Example 1. Formulation (b) can be prepared, for example, by initially obtaining or preparing paclitaxel in a 1:1 dilution of ethanol and Cremophor EL® (Sigma, St. Louis, Mo.), and then adding saline or 5% glucose to prepare paclitaxel in 5% w/v ethanol and 5% w/v Cremophor. Formulation (c) contains the same final concentrations of serum albumin as formulation (a) in addition to the same final concentrations of paclitaxel, ethanol and Cremophor as formulation (b). In each test, formulations (a), (b) and (c) should comprise the same final concentrations of paclitaxel and be administered in equal volumes. Formulations (d) consist of various controls which comprise: all the components of formulation (a) except the paclitaxel; all the components of formulation (b) except paclitaxel; or all the components of formulation (c) except paclitaxel. It is expected that a formulation comprising paclitaxel and serum albumin would be as effective and less toxic than a formulation comprising paclitaxel and Cremophor.

Various dosages of paclitaxel are used, from 0.3 to 30 mg/kg body weight. The paclitaxel formulations and controls (a) to (d) can be administered as a bolus (single injection) or as a drip over a period of 15 minutes or less, or about 150 min or less. Each combination of paclitaxel formulation and control (a) to (d) and cancer cell type is tested on a group of about 20 animals.

Twice weekly after injection of paclitaxel formulation or control, the animals are examined for treatment. Efficacy of treatment can be monitored by detection of serum levels of tumor-specific antigens, by histological analysis, or by physical measurement of tumor size. Serum levels can be examined by testing blood samples with labeled antibodies specific for tumor-associated antigens. Histological analysis can be performed by sacrificing the animals and microscopically analysing tissues. Tumor number and size can be determined with calipers. Successful treatment is indicated by lack of tumor expansion or tumor shrinkage; or maintenance of serum antigen levels, or decrease of serum antigen levels.

Side effects such as neutropenia, peripheral neuropathy, and anemia, typical of paclitaxel treatment, are monitored. Levels of side effects can also be monitored with animals treated as described above, but also pre-treated (prior to injection with paclitaxel formulation) with corticosteroids, diphenhydramine, H₂ antagonists, and/or other agent known to reduce to side effects of paclitaxel.

In addition, cancer cells of any type can be substituted for breast or ovarian cancer cells in the protocol described above, in order to test the efficacy of paclitaxel formulations of the present invention against such a cancer.

### EXAMPLE 3

Animal tests for the efficacy of compositions of paclitaxel, serum albumin and a physiologically acceptable vehicle in treating rheumatoid arthritis, systemic lupus erythematosus, parasitic infections, and restenosis

Briefly, the efficacy of paclitaxel formulations of the present invention against various diseases such as rheumatoid arthritis, lupus erythematosus, and parasitic infections can be tested using the test animals and the protocols described herein.

3. A. Animal Tests for Treating Rheumatoid Arthritis

In order to test the efficacy of present paclitaxel formulations against rheumatoid arthritis, a collagen-induced arthritis model system can be used. Syngeneic female Louvain (LOU) rats are injected, under anaesthesia, with 0.5 mg of native chick collagen type II (CII) (Genzyme, Boston, Mass.) solubilized in 0.1 M acetic acid and emulsified in IFA (Difco, Detroit, Mich.) Trentham et al. (1977) J. Exp. Med. 146:857-868. Between 90-100% of rats typically develop synovitis by day 9 post-immunization.

Paclitaxel formulations and controls (a) to (d) described in Example 2 are then injected into the animals as described in Example 2.

The incidence and severity of arthritis is measured daily following injection. Incidence is measured by the number of rats with clinical evidence of joint inflammation. Severity of inflammation of each paw is evaluated using an integer scale from 0 to 4. Delayed type hypersensitivity (DTH) can also be determined by radiometric ear assay. Trentham et al. (1980) Arthritis Rheum. 23:932-936. Efficacy of treatment is indicated by a stabilization or reduction in incidence or severity of inflammation.

3. B. Animal Tests for Treating Systemic Lupus Erythematosus

The efficacy of paclitaxel formulations of the present invention in treating systemic lupus erythematosus (SLE) can be tested in various animal models, including NZB/NZW mice and MRLI/I mice. The former particularly spontaneously develop autoimmune diseases closely paralleling systemic lupus erythematosus and are particularly useful for studying mortality and kidney malfunctions associated with SLE. The latter are particularly suited for studying arthritis and anti-SM antibodies in SLE. Adelman et al. (1983) J. Exp. Med. 158: 1350-1355; Knight et al. (1978) J. Exp. Med. 147: 1653; Theofilopoulos et al. (1980) Clin. Immunol. Immunopathol. 15:258-278; and Theofilopoulos (1985) Adv. Immunol. 37:269-390.

The test animals, such as MRL mice, spontaneously develop autoimmune disease and massive nonmalignant T cell proliferation that kills 50% of them by 5 to 6 months of age. At 3 months of age, animals are tested for the disease progression and then injected with paclitaxel formulations and controls (a) to (d) as described in Example 2. Animals are then monitored for disease progression and death. Efficacy of treatment is measured by amelioration of symptoms or extended lifespan (beyond 5 to 6 months). Disease progression can be monitored by, for example, determining serum levels of a heavy form of gp70 protein. This gp70 varies in sedimentation rates from 9S to 19S in sucrose density gradient analysis and appears with the onset of disease and persists throughout its course.

3. C. Animal Tests for Treating Parasitic Diseases

Paclitaxel formulations of the present invention can be tested for efficacy in treating parasitic diseases such as those caused by organisms of the Plasmodia, Trypanosoma or Babesia genuses, using either in vitro tests with infected human erythrocytes or in vivo tests with infected rats, or directly testing the formulations against cultures of parasites. Baum et al. (1981) Proc. Nail. Acad. Sci. U.S.A. 78:4571-4575; U.S. Pat. No. 5,631,278.

Direct tests against cultures of parasites comprises treating such cultures with 1 μM to 10 μM solutions of paclitaxel formulations or controls (a) to (d) described above and determining the effect on parasite viability.

In in vitro tests, cultures of human erythrocytes are infected with parasites and grown in the presence of paclitaxel or controls (a) to (d) described above. Infections are synchronized with sorbitol treatment to +2 hours. Lambros et al. (1979) Parasitol. 65:418-420. Paclitaxel formulations are added 4 hours post-invasion and maintained in cultures for three life cycles; each life cycle is approximately 48 hours long. Parasitemia is measured by examining Giemsa-stained blood smears using a light microscope. Efficacy is measured by determining the proportion of blood cells infected with parasites. Successful treatment is indicated by a reduction in this proportion.

In in vivo-tests, mice are injected with parasites and, once parasitaemia was achieved, injected with paclitaxel formulations. More specifically, for example, six- to eight-week-old mice, such as BALB/c mice (Jackson Laboratories) are administered intraperitoneal injections of 2×10⁵ Plasmodium chabaudi adami parasites. Parasitaemia is estimated by tail vein blood smears, and allowed to reach a level wherein 1% to 2% of blood cells are infected. This requires seven to ten days. Paclitaxel formulations or controls (a) to (d) described above are then injected into the mice as described above. Daily smears are then tested over eight or more parasite life cycles to monitor disease progression. Again, successful treatment is indicated by a reduction of the proportion of blood cells infected with parasites.

3. D. Animal Tests for Treating Restenosis

In determining the efficacy of paclitaxel formulations of the present invention in treating restenosis, test animals are subjected to arterial damage and then treated. Ferns et al. (1991) Science 253:1129-1132. More specifically, test animals (such as Wistar rats) are anesthetized with pentobarbital [20 mg/kg body weight (b.w.)], ketamine (2 mg/kg b.w.), and xylazine (4 mg/kg b.w.) intraperitoneally. An artery such as the left external carotid artery is cannulated with 2-French Fogarty embolectomy catheter, inflated with saline and passed three times up and down the common carotid artery to produce a distending, de-endothelializing injury. The animals are treated with paclitaxel formulations or controls (a) to (d) described above beginning two hours after the injury. After one and a half weeks, the animals are sacrificed, and the carotid arteries removed and fixed in 10% vehicled formalin and embedded in paraffin. Cross-sections of the carotids are examined microscopically and stained with hematoxylin and cosin stain. Successful treatment is indicated by reduction of the neointimal area.

In the assays described in these Examples, the tested animals can also be monitored for side effects, as described in Example 2, in order to determine the toxicity of various paclitaxel formulations and controls.

### EXAMPLE 4

Administration of a Pre-Treatment Agent and a Paclitaxel Formulation

Pre-Treatment

Prior to administration to a patient of a paclitaxel formulation described in Example 1, a pre-treatment agent can be administered. Such a pre-treatment agent is capable of reducing side effects associated with paclitaxel administration.

Briefly, the pretreatment agent can be administered less than about 48, 24, 12, 6, 3 or 1 hours prior to administration of paclitaxel. The pre-treatment agent can be Dexamethasone (20 mg), administered about 14 to about 12 hours and about 7 to about 6 hours prior to paclitaxel administration; Ranitidine (50 mg) or famotidine (20 mg) administered 30 minutes prior to paclitaxel administration; Cimetidine (300 mg) and Diphenhydramine (25 to 50 mg) administered 30 minutes prior to paclitaxel administration; or G-CSF (5 mg/kg/day), administered prior to paclitaxel administration. Regular (daily, twice-weekly, weekly, tri-weekly) administrations of the pre-treatment agent can be performed during and after administration of the paclitaxel formulation.

Paclitaxel Formulation Administration

The paclitaxel formulation described in Example 1 can be administered in varying dosages. A single dosage can be at least about 100 or at least about 200 mg/m². The single dosage can be less than about 300 mg/m². The final concentration of administered paclitaxel can be between about 0.3 to about 1.2 mg/ml. The paclitaxel can be administered as a drip in a duration of less than about 24, 18, 12, 6, 3, or 1 hours, or less than about 15 minutes. These administrations can be repeated every week, every two weeks, or every three weeks. Repeated administrations can continue for six to eighteen months. Repeated administrations can also be preceded, accompanied or followed by administrations of a pre-treatment agent. The patient should be monitored throughout treatment for efficacy of treatment and appearance of side effects. Administration of paclitaxel should be discontinued and medical treatment obtained should side effects appear.

### EXAMPLE 5

Drying and Reconstitution of Compositions of Paclitaxel and Serum Albumin

The composition comprising paclitaxel, a serum albumin and a physiologically acceptable vehicle of the present invention can be dried, stored as a dried composition, and then resolubilized prior to administration. The drying process can be performed by any method known in the art, including lyophilization. The composition prior to drying can comprise a physiologically acceptable vehicle, such as McIlvaine buffer. The composition can be stored as a dried composition. The composition can be reconstituted after lyophilization with a physiologically acceptable vehicle, such as McIlvaine buffer, water or certain saline solutions, including dilutions of saline.

Experiments have been conducted on the effect of lyophilization and resolubilization of human serum albumin (HSA)-bound paclitaxel (Ptx). Two preparations of Ptx-HSA were made in McIlvaine buffer solutions containing 5% ethanol. The final pH of the preparations was 3.3 and 7.2. The molar ratio of Ptx to HSA was 1:1, with the concentrations of Ptx and HSA kept at 200 μg/mL and 15.6 mg/mL, respectively. A successful reconstitution of this formulation was achieved with acidic preparations of Ptx-HSA in final concentrations of up to 200 μg/mL Ptx, when the resolubilization was carried in McIlvaine buffer solutions. Surprisingly, on the basis of the ELISA data and contrary to the turbidity data, not only the acidic but also the neutral preparations of Ptx-HSA could be resolubilized in WFI (water for injection) alone or supplemented with an additive.

Attempts were also made to resolubilize Ptx-HSA at a Ptx concentration greater than 200 μg/mL. At present, clear solutions of 400 μg/ml Ptx could be obtained as well as 1000 μg/mL. However, the stability of the later was very limited, on the basis of turbidity.

This study also consisted of a first attempt to partly scale-up the standard 2-mL reaction mixture in a test tube to a 50-mL reaction mixture in a beaker.

Future studies will determine the reproducibility of the these experiments, and assess the effect of different solubilization vehicles before and post-lyophilization to determine the optimal resolubilization conditions. Also in future studies, radiolabelled Ptx will be used to obtain quantitative measure of the binding.

5. A. Effect of Resolubilization of Ptx-HSA in McIlvaine Buffer and WFI Solutions

To be practical and of greater shelf life, the final Ptx-HSA formulation can be in a dried form, such as a lyophilized form. Consequently, it was necessary to test the effect of drying on the product as well as its successful reconstitution into a clear solution for administration.

5. A. 1. Objective

This present study was designed to evaluate the solubility of two lyophilized HSA-bound Ptx preparations in McIlvaine buffer and WFI solutions.

5. A. 2. Experimental Procedure

Two preparations of Ptx-HSA were made by binding Ptx to HSA, added at concentrations of 200 μg/mL and 15.6 mg/mL, respectively, to maintain a molar ratio of 1:1. One of the preparations was made in 5% ethanol in McIlvaine buffer, pH 3.0. The actual (final) pH of this reaction mixture was 3.3. The other preparation was made in the same solution but at a pH of 7.2. Fifty milliliters of each preparation were lyophilized and portions of which were tested for successful resolubilization under different conditions.

5. A. 3. Results and Conclusions

The present study represented the first attempt to partly scale-up the formulation mixture of Ptx and HSA from the standard 2-mL solution in a test tube to a 50-mL solution, in a beaker. The mixing was achieved with the aid of a magnetic stirrer, as opposed to vortexing. The two preparations of Ptx-HSA (pH 3.3 and 7.2) were analyzed qualitatively, and found to be clear after a 1-hour incubation. The reaction mixtures were clarified by centrifugation then lyophilized.

Following the freeze-drying, the weight of two preparations of Ptx-HSA was measured and the results are shown in Table 22. The amount of Ptx in both pH 3.3 and 7.2 preparations of Ptx-HSA was small, representing only 0.6 and 0.5% of the total weight, respectively. Portions of these preparations were resolubilized in McIlvaine buffer solutions of pH 3.0 and 7.2, alone or supplemented with ethanol at 5 and 20% (v/v). Generally, the conditions that showed good recovery were those associated with the pH 3.0 vehicle solution, used either before or/and after the lyophilization (Table 23).

It should be noted that in this study, the salt content of the resolubilized Ptx-HSA preparation was increased, as a result of using the same vehicle solutions in the preparation of the material for lyophilization and during the resolubilization.

TABLE 22
Estimation of Ptx, HSA and salts concentrations in the
freeze-dried preparations of Ptx-HSA.

| | Dry-weight per | % HSA | % salts | % Ptx |
|---|---|---|---|---|
| Sample name | 50-mL (mg) | (w/w) | (w/w) | (w/w) |
| Ptx-HSA, pH 3.3 | 1608 | 48.36 | 51.02 | 0.62 |
| Ptx-HSA, pH 7.2 | 1963 | 39.78 | 59.71 | 0.51 |

TABLE 23
Recovery of soluble Ptx after lyophilization and resolubilization
in McIlvaine buffer solutions under different conditions.

| | Ptx | | | |
|---|---|---|---|---|
| | conc. | | % | |
| | (μg/ | | Bound | |
| Sample name | mL) | Resolubilization vehicle | Ptx | % CV |
| Ptx-HSA, pH 3.3 | 200 | 5% EtOH, pH 3.0 vehicle | 116.4 | 4.3 |
| Ptx-HSA, pH 7.2 | 200 | 5% EtOH, pH 3.0 vehicle | 91.8 | 8.6 |
| Ptx-HSA, pH 3.3 | 200 | 5% EtOH, pH 7.2 vehicle | 68.5 | 27.1 |
| Ptx-HSA, pH 7.2 | 200 | 5% EtOH, pH 7.2 vehicle | 54.1 | 22.2 |
| Ptx-HSA, pH 3.3 | 200 | 20% EtOH, pH 3.0 vehicle | 77.4 | 3.0 |
| Ptx-HSA, pH 7.2 | 200 | 20% EtOH, pH 3.0 vehicle | 50.8 | 12.9 |
| Ptx-HSA, pH 3.3 | 200 | 20% EtOH, pH 7.2 vehicle | 100.7 | 35.6 |
| Ptx-HSA, pH 7.2 | 200 | 20% EtOH, pH 7.2 vehicle | 17.1 | 16.2 |
| Ptx-HSA, pH 3.3 | 50 | 0% EtOH, pH 3.0 vehicle | 89.5 | 11.9 |
| Ptx-HSA, pH 7.2 | 50 | 0% EtOH, pH 3.0 vehicle | 93.9 | 24.9 |
| Ptx-HSA, pH 3.3 | 50 | 0% EtOH, pH 7.2 vehicle | 88.9 | 11.4 |
| Ptx-HSA, pH 7.2 | 50 | 0% EtOH, pH 7.2 vehicle | 164.7 | 8.9 |

Note:
The Ptx concentration is an estimate based on the starting Ptx concentration in the pre-lyophilization solutions. An amount of lyophilized Ptx-HSA was dissolved in the vehicle to give the estimated Ptx concentration.

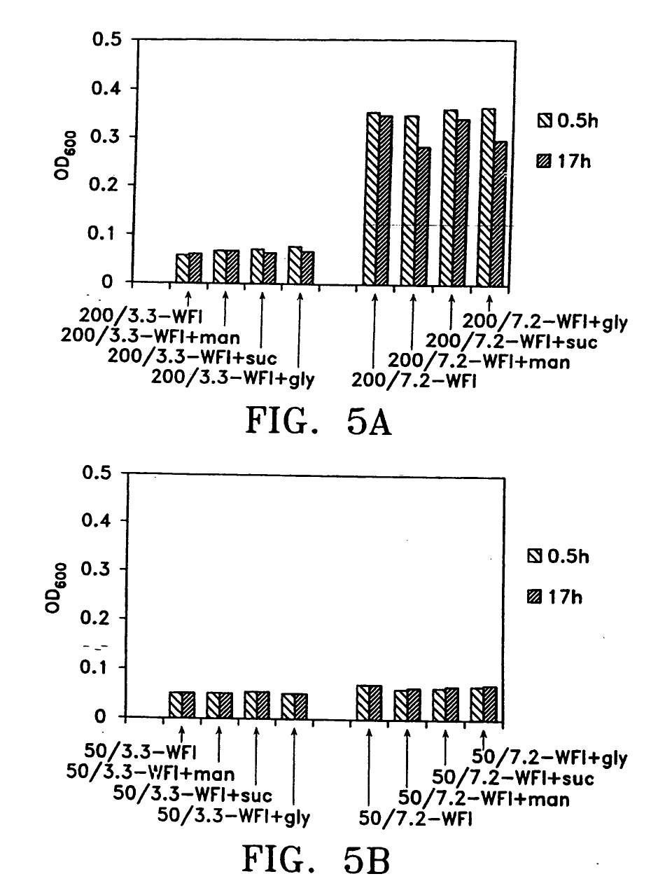

To avoid changes in salt concentration in the final formulation, the experiment was repeated using WFI alone or with additive to resolubilize the freeze-dried Ptx-HSA (Table 24). Surprisingly, all of the conditions tested showed good recovery of soluble Ptx with both preparations, and at either 50 or 200 μg/mL of Ptx. The recovery with the pH 7.2 Ptx-HSA preparation was lower at 200 μg/mL of Ptx with most of the resolubilization conditions tested, consistent with the turbidity data (FIG. 5). When these samples were clarified by centrifugation, significant amount of precipitate collected at the bottom of the tubes. Based on the recovery of about 80% soluble Ptx in the pH 7.2 Ptx-HSA solutions at 200 μg/mL of Ptx and the observed amount of precipitate, it can be concluded that the turbidity was partly due to insoluble salts, and presumably to Na₂HPO₄.

TABLE 24
Recovery of soluble Ptx after lyophilization and resolubilization
in WFI under different conditions.

| | Ptx | | Solu- | % | |
|---|---|---|---|---|---|
| | conc. | Resolubilization | tion | Bound | % |
| Sample name | (μg/mL) | vehicle | pH | Ptx | CV |
| Ptx-HSA, pH 3.3 | 200 | WFI | 3.3 | 102.7 | 14.5 |
| Ptx-HSA, pH 3.3 | 200 | 1% mannitol in WFI | 3.3 | 113.0 | 9.9 |
| Ptx-HSA, pH 3.3 | 200 | 1% sucrose in WFI | 3.3 | 119.7 | 5.9 |
| Ptx-HSA, pH 3.3 | 200 | 1% glycerol in WFI | 3.3 | 129.3 | 14.0 |
| Ptx-HSA, pH 7.2 | 200 | WFI | 7.2 | 77.2 | 15.3 |
| Ptx-HSA, pH 7.2 | 200 | 1% mannitol in WFI | 7.2 | 102.3 | 19.0 |
| Ptx-HSA, pH 7.2 | 200 | 1% sucrose in WFI | 7.2 | 81.5 | 7.5 |
| Ptx-HSA, pH 7.2 | 200 | 1% glycerol in WFI | 7.2 | 80.3 | 1.1 |
| Ptx-HSA, pH 3.3 | 50 | WFI | 3.3 | 126.6 | 5.1 |
| Ptx-HSA, pH 3.3 | 50 | 1% mannitol in WFI | 3.3 | 135.3 | 11.4 |
| Ptx-HSA, pH 3.3 | 50 | 1% sucrose in WFI | 3.4 | 131.4 | 7.1 |
| Ptx-HSA, pH 3.3 | 50 | 1% glycerol in WFI | 3.4 | 108.0 | 3.3 |
| Ptx-HSA, pH 7.2 | 50 | WFI | 7.2 | 128.4 | 5.3 |
| Ptx-HSA, pH 7.2 | 50 | 1% mannitol in WFI | 7.2 | 129.3 | 1.5 |
| Ptx-HSA, pH 7.2 | 50 | 1% sucrose in WFI | 7.2 | 127.4 | 4.6 |
| Ptx-HSA, pH 7.2 | 50 | 1% glycerol in WFI | 7.2 | 154.9 | 0.7 |

Note:
The Ptx concentration is an estimate based on the starting Ptx concentration in the pre-lyophilization solutions. An amount of lyophilized Ptx-HSA was dissolved in the vehicle to give the estimated Ptx concentration.

This study also suggested that the addition of mannitol or other tested additives to WFI was not necessary, under these experimental conditions.

5. B. Effect of Resolubilization of Ptx-HSA in WFI Solutions at Ptx Concentration Greater than 200 μg/ml

Thus far, the reconstitution study was designed to resolubilize Ptx in solution of up to 200 μg/mL Ptx, not to exceed its concentration in the pre-lyophilization reaction mixture.

5. B. 1 Objective

This present study was designed to evaluate the solubility of two lyophilized HSA-bound Ptx preparations in WFI solutions, at Ptx concentration greater than 200 μg/mL.

5. B. 2 Experimental Procedure

The procedure is as in section 5. A. 2.

5. B. 3 Results and Conclusions

Portions of the two Ptx-HSA preparations were resolubilized in WFI supplemented with mannitol (1%, w/v), to give an estimated Ptx concentration of 400 and 1000 μg/mL. As with the resolubilization of 200 μg/mL of Ptx, the acidic preparation were clearer than the neutral pH preparations. Quantitation of binding was estimated by ELISA, and the results are shown in Table 25. It is evident that a soluble preparation of Ptx-HSA can be obtained with a Ptx concentration of at least 400 μg/mL. More studies are needed to establish the upper limit and the reproducibility of the results.

TABLE 25
Recovery of soluble Ptx after lyophilization and resolubilization
in WFI with 1% mannitol.

| Sample | Ptx conc. | Resolubilization | Solution | % Bound | % |
|---|---|---|---|---|---|
| name | (μg/mL) | vehicle | pH | Ptx | CV |
| Ptx-HSA, | 400 | 1% mannitol in WFI | ND | 101.7 | 12.7 |
| pH 3.3 | | | | | |
| Ptx-HSA, | 1000 | 1% mannitol in WFI | ND | ND | ND |
| pH 3.3 | | | | | |
| Ptx-HSA, | 400 | 1% mannitol in WFI | ND | 70.7 | 0.7 |
| pH 7.2 | | | | | |
| Ptx-HSA, | 1000 | 1% mannitol in WFI | ND | 93.0 | 5.1 |
| pH 7.2 | | | | | |

Note:
The Ptx concentration is an estimate based on the starting Ptx concentration in the pre-lyophilization solutions. An amount of lyophilized Ptx-HSA was dissolved in the vehicle to give the estimated Ptx concentration.
ND: not determined.

5. C. Additional Reconstitution Studies

Reconstitution Studies (Exp. # 38, 44, 50)

The candidate NBI Ptx/HSA formulations have an acidic pH, a condition which has been established as optimal for the binding of Ptx to HSA. The formulation mixtures consisted of Ptx and HSA added at 1:2 molar ratio, in 4% aqueous ethanolic acidic solutions. Different reconstitution conditions of the candidate lyophilized NBJ Ptx/HSA formulations have been evaluated for stability to select a product suitable for injection.
i) Experimental objectives and rationale:
   Reconstitution of stable Ptx/HSA formulations.
   Analysis of the effect of different HSA preparations and buffer systems on the recovery and binding after reconstitution.
ii) Experiment:
   Ptx was formulated with different preparations of HSA:
   HSA-A, neutral undefatted.
   HSA-B, acidic undefatted.
   HSA-C, neutral defatted.
   HSA-D, acidic defatted.
   Ptx/HSA molar ratios tested: 1:2.
   Ptx concentration was fixed at 200 μg/mL, typically, but was also varied up to 600 μg/mL, with final ethanol concentration of 4%.
   Buffer systems for binding or reconstitution:
   McIlvaine buffer solutions.
   Saline solutions.
   Glycine/NaOH, TEA/NaOH, and WFI.
      iii) Results and Conclusion:
Reconstitution of all acidic formulations with WFI were clear (OD₆₀₀ values of less than 0.1). No difference was observed between formulations prepared with undefatted and defatted HSA. HPLC analysis showed good recovery of soluble Ptx, after extraction of Ptx bound to HSA with tert.-butyl methyl ether according to a procedure by (Sharma et al., 1994).
   However, lyophilized formulations made with neutral pH HSA (defatted and undefatted) were found to resolubilize fasted than the formulations made with the acidic pH HSA HSA (defatted and undefatted).
   All 4 types of HSA have yielded stable reconstituted acidic formulation after 24 h of storage at both 4° C. and room temperature.
   Quantitative analysis of recovery of soluble Ptx has been carried out using radioactive Ptx.
   Qualitative analysis of soluble Ptx has been carried out by reverse-phase HPLC, and showed no degradation product associated with the acid formulations.
   Attempt to shift the pH from acidic to neutral pH resulted in cloudy solutions of varying degree, presumably due to precipitation of Ptx (Table 26). Degradation products were observed at alkaline pH.
   Formulation mixtures were also evaluated for presence of filtrable particulates using a 0.2 micron cellulose acetate filter. No detectable difference in recovery was observed from this treatment for the acidic formulation. However, increasing the pH of the formulations resulted in lower recovery of soluble Ptx (Table 6).

Preliminary studies evaluating different buffer solutions (TEA, Glycine and McIlvaine) and buffer concentrations for reconstitution showed promising results (study in progress).

TABLE 26
Stability of lyophilized Ptx/HSA formulations of different pH after
reconstitution and storage at room temperature for 24 h.

| | | | After reconstitution | | Day 1 storage at 23° C. | |
|---|---|---|---|---|---|---|
| HSA | | | Total | Estimated | Total | Estimated |
| excess | | | soluble Ptx | HSA | soluble Ptx | HSA |
| molar | Formulation | Ptx conc | Recovery⁽¹⁾ | bound | Recovery | bound Ptx |
| amount | pH | (μg/mL) | (%) | Ptx (%) | (%) | (%) |
| 2 | 3.5 | 200 | 100.2 | 93.5 | 97.6 | 94.6 |
| 2 | 4.8 | 200 | 81.3 | 76.6 | 75.1 | 72.8 |
| 2 | 6.1 | 200 | 73.3 | 57.7 | 68.5 | 47.6 |
| 2 | 7.1 | 200 | 64.7 | 52.3 | 60.9 | 55.1 |

¹⁾Total soluble Ptx consists of HSA-bound Ptx and unbound Ptx in solution, estimated after removal of insoluble Ptx. The results are averages of triplicate data points.

TABLE 27
Evaluation of the effect of microfiltration on the recovery of soluble Ptx in
reconstituted formulations of different pH.

| | | | After reconstitution | | |
|---|---|---|---|---|---|
| HSA | | | Total soluble | Total soluble Ptx | Estimated |
| excess | | Ptx | Ptx Recovery | Recovery after | HSA |
| molar | Formulation | conc | after centrifugation⁽¹⁾ | microfiltration⁽²⁾ | bound Ptx |
| amount | pH | (μg/mL) | (%) | (%) | (%) |
| 2 | 3.5 | 200 | 100.2 | 93.5 | 92.7 |
| 2 | 4.8 | 200 | 81.3 | 62.4 | 57.7 |
| 2 | 6.1 | 200 | 73.3 | 45.5 | 29.9 |
| 2 | 7.1 | 200 | 64.7 | 33.4 | 20.9 |

¹⁾Total soluble Ptx consists of HSA-bound Ptx and unbound Ptx in solution, estimated after removal of insoluble Ptx by centrifugation. The results are averages of triplicate data points.
²⁾Total soluble Ptx consists of HSA-bound Ptx and unbound Ptx in solution, estimated after removal of insoluble Ptx by microfiltration using a 0.2 micron cellulose acetate filter. The results are averages of triplicate data points.

1.2 Effect of Scale-Up (Exp. # 61).
i) Experimental objective and rationale:
   Determine the effect of scale-up the formulation for lyophilization from 3 to 100 mL per vial.

Evaluation of scaling-up two different concentrations of Ptx: 200 and 300 mg/mL.

TABLE 28
Evaluation of the effect of the Ptx/HSA formulation scale-up on the binding
and recovery of soluble Ptx before and after lyophilization.

| | | | Day 0 post - | | Day 1 post - | |
|---|---|---|---|---|---|---|
| Formulation conditions | | | reconstitution | | reconstitution | |
| | | Total Ptx | Total | Estimated | Total | Estimated |
| Scale-up | | amount | soluble Ptx | HSA | soluble Ptx | HSA |
| volume | Ptx conc | per vial | Recovery⁽²⁾ | bound | Recovery | bound Ptx |
| (mL) | (μg/mL) | (mg) | (%) | Ptx (%) | (%) | (%) |
| 3 | 200 | 0.9 | 100.6 | 92.6 | 94.9 | 85.4 |
| 30 | 200 | 9 | 99.9 | 91.4 | 91.8 | 82.5 |
| 100 | 200 | 30 | 99.0 | 91.0 | 79.4 | 59.2 |
| 75 | 200 | 15 | 98.6 | 87.7 | 102.1 | 93.4 |

5. D. Effect of Polyols of the Stability of the Lyophilized Formulation

1.3 Effect of Polyols on Stability of Lyophilized Formulation (Exp. #55).

Evaluation of the Effect of Polyols on Lyophilization of Ptx/HSA Formulations.

Materials
3.1 Antioxidant solutions:
   3.1.3 Dithioerythritol: 400 mM stock solution in WFI.
   3.1.4 Cysteine: 400 mM stock solution in WFI.
3.2 Commercial HSA solution (20%).
3.3 A fresh paclitaxel stock solution: 5 Ptx (5 mg/mL) in dehydrated EtOH, with radioactive Ptx at 1/200 dilution. The ethanol solution must be dehydrated.
3.4 Binding buffer solutions:
   1. 1× McIlvaine, pH 3.0.
   2. 1× McIlvaine, pH 3.0, with 1.5% sorbitol.
   3. 1× McIlvaine, pH 3.0, with 3% sorbitol.
   4. 1× McIlvaine, pH 3.0, with 6% sorbitol.
   5. 1× McIlvaine, pH 3.0, with 1.5% mannitol.
   6. 1× McIlvaine, pH 3.0, with 3% mannitol.
   7. 1× McIlvaine, pH 3.0, with 6% mannitol.
   Filter sterilize (0.2 micron cellulose filters) or autoclave.
3.4 Also prepare sterile flasks or beakers of appropriate size for mixing the formulations. 1 flask or beaker/condition.

Procedure
4.1 Prepare an antioxidant solution consisting of a mixture of cysteine and DTE (200 mM each) as follows:
   4.1.1 400 mM cysteine solution. 96.96 mg of cysteine in 2 mL of WFI.
   4.1.2 400 mM DTE solution. 123.3 mg of DTE in 2 mL of WFI.
   4.1.3 Add 1 mL of 400 mM cysteine to 1 mL of 400 mM DTE to make a solution of 200 mM cysteine+200 mM DTE.
4.2 Pre-treat a 20% commercial HSA solution with DTE and cysteine (4 mM each) overnight at two HSA solutions in 15-mL conical tubes labeled as follows (HSA+DTE/Cys): add 38.8 mL of 20% HSA and 0.792 mL of DTE+Cys solution (200 mM prepared in 4.1.3). Note the concentration of DTE and Cys in the HSA solutions would be 4 mM each.
4.3 Incubate the HSA+antioxidant solution overnight, at 2-8° C.
4.4 After incubation acidify the solution to 3.1-3.3 with 0.85% phosphoric acid dilute the acidified HSA solution with WFI to make a 10% HSA-B solution containing 2 mM each of DTE and cysteine.

4.5 Pre-label 42 sterile 20-mL serum vials for later use to aliquot the formulation solutions for lyophilization. Label the samples (6 vials/condition) with the Exp. # 55-0, vial labeling information below, date, and investigator's initials.

| Condition | Comments | Vial labeling |
|---|---|---|
| 1. PH23-4.200: 2B/3.0 | 0% polyol | 1-1 to 1-6 |
| 2. PH23-4.200: 2B/3.0 | 1% sorbitol | 2-1 to 2-6 |
| 3. PH23-4.200: 2B/3.0 | 2% sorbitol | 3-1 to 3-6 |
| 4. PH23-4.200: 2B/3.0 | 4% sorbitol | 4-1 to 4-6 |
| 5. PH23-4.200: 2B/3.0 | 1% mannitol | 5-1 to 5-6 |
| 6. PH23-4.200: 2B/3.0 | 2% mannitol | 6-1 to 6-6 |
| 7. PH23-4.200: 2B/3.0 | 4% mannitol | 7-1 to 7-6 |

Reaction Conditions (Apply Aseptic Techniques Whenever Possible).
5.1 Ptx concentration: 200 μg/mL, with radioactive Ptx.
5.2 HSA concentration: 31.2 mg/mL.
5.3 Molar ratio: 1:2.
5.4 EtOH concentration: 4%.
5.5 Binding conditions (in 100-mL beaker or Erlenmeyer flask, with stirring).
   Note: each vial will contain 3 mL of formulation solution.

(i) Prepare each of the formulation mixtures above at 23° C., by adding HSA first, then buffer followed by Ptx/EtOH.

| | (20 mL solutions) |
|---|---|
| 10% HSA-B | 6.24 mL |
| Buffer | 12.96 mL |
| 5 Ptx | 0.8 mL |

Ensure continuous mixing during addition of Ptx/EtOH.
(ii) Remove 1 mL of each formulation for analysis of recovery and binding before lyophilization by LSC.
(iii) Centrifuge the reaction mixtures, 20 min, at 3400 rpm in the IEC centrifuge.

Lyophilization Conditions
6.4 Aliquot 3 mL of each formulation (200 μg/mL Ptx, 1:2 molar ratio, 4% EtOH, with or without polyols and TWEEN) in labeled 10-mL serum vials.
6.5 Give samples to Ted before 16:00, with rubber stoppers loosely placed on the vials, not restrict vapor flow during lyophilization.
6.6 After lyophilization do a wipe test for radiation monitoring of the lyophilizer.

Reconstitution Conditions and Analysis
6.1 Reconstitute the powder aseptically in 3 mL of WFI, in triplicates. Observe the samples for the facility to reconstituted
   Appearance of the powder after addition of WFI.
   Length of time to complete dissolution.
6.2 Allow the reconstituted samples to incubate for at least 0.5 h and then analyze the recovery and binding by LSC.
   8. Day 0: R, S and F.
   9. Day 1: S and F.
6.3 Collect data for processing and analysis by excel.
   7.3.1 Day 0: R, S and F for the formulation before and after lyophilization (reconstituted formulation).
   7.3.2 Day 1: S and F for the formulation after lyophilization (reconstituted formulation).

Exp. # 55.
ii) Experimental objective and rationale:
   Determine the effect of polyols on the stability of the Ptx/HSA formulation before and after the lyophilization and reconstitution.
iii) Experimental objective and rationale:
   Determine the effect of polyols on the stability of the Ptx/HSA formulation before and after the lyophilization and reconstitution.

(iv) Results and conclusion:

TABLE 29a
Effect of polyols on the stability of liquid and reconstituted lyophilized
Ptx/HSA formulation.

| | Day 0 | | Day 1 | | Day 3 | |
|---|---|---|---|---|---|---|
| | % | | % | | % | % |
| Polyol | Recovery | % CV | Binding | % CV | Recovery | CV |
| 0% polyol | 88.47 | 5.60 | 98.01 | 2.01 | 103.86 | 2.00 |
| 1% sorbitol | 86.33 | 3.49 | 90.77 | 3.71 | 104.09 | 5.49 |
| 2% sorbitol | 74.69 | 2.93 | 88.84 | 1.47 | 101.00 | 0.75 |
| 4% sorbitol | 74.69 | 2.93 | 88.84 | 1.47 | 101.00 | 0.75 |
| 1% mannitol | 86.33 | 3.49 | 90.77 | 3.71 | 104.09 | 5.49 |
| 2% mannitol | 74.69 | 2.93 | 88.84 | 1.47 | 101.00 | 0.75 |
| 4% mannitol | 74.69 | 2.93 | 88.84 | 1.47 | 101.00 | 0.75 |

TABLE 29b
Effect of polyols on the stability of reconstituted lyophilized Ptx/HSA
formulation.

| | Day 0 | | Day 1 | | Day 3 | |
|---|---|---|---|---|---|---|
| Polyol | % Recovery | % Binding | % Recovery | % Binding | % Recovery | % Binding |
| 0% polyol | 92.4 | 89.4 | 85.7 | 82.5 | 65.6 | 60.2 |
| 1% sorbitol | 92.4 | 89.1 | 65.3 | 59.8 | 25.6 | 22.9 |
| 2% sorbitol | 102.4 | 99.4 | 98.4 | 95.1 | 95.3 | 91.6 |
| 4% sorbitol | 98.8 | 96.1 | 97.9 | 93.9 | 97.8 | 93.3 |
| 1% mannitol | 99.2 | 95.6 | 97.7 | 93.6 | 96.6 | 92.2 |
| 2% mannitol | 99.4 | 94.8 | 97.2 | 90.0 | 89.1 | 82.0 |
| 4% mannitol | 98.9 | 95.0 | 77.9 | 72.3 | 16.5 | 14.8 |

1.4 Effect of Microfiltration on the Recovery of Ptx in the HSA Formulation.
   1.4.1 Effect of different filters: nylon and surfactant free cellulose acetate (SFCA), on the recovery.
   1.4.2 Effect of repeated filtration of equal volume of formulation using one filter, to estimate the filter saturation.
   1.4.3 Effect of continuous filtration of same formulation solution in different filters, to estimate binding capacity.

Summary:
viii) Experimental objectives and rationale:
   For pre-clinical work, at times a filter-sterilization step may be required for non-sterile lyophilized formulations, prior to use. For commercial formulations the sterilization is required prior to filling to have an acceptable lyophilized product. In this study, two types of 0.2 micron filters (nylon and SFCA) were evaluated for:
      Potential use in the sterilization of the formulation prior to filling.
      Determining whether the filters bind the product and establish its saturation level.
      Determining whether there were any filterable precipitates before lyophilization or after reconstitution.
         ix) Experiment:
Liquid and reconstituted lyophilized Ptx/HSA formulations were analyzed.
Ptx concentration was 200 μg/mL and HSA-B added at 1:2 molar ratio.
   x) Results and Conclusion:
Both nylon and SFCA filters bound Ptx/HSA
The binding was saturable.
Precipitable material removed by centrifugation was not completely removed by microfiltration, as evidenced by the recovery at pH 7.0 (panel C).

Although the foregoing invention has been described in some detail by way of illustration and example for purposes of clarity of understanding, it will be apparent to those skilled in the art that certain minor changes and modifications will be practiced. Therefore, the description and examples should not be construed as limiting the scope of the invention, which is delineated by the appended claims.

1. An optically clear, pharmaceutically acceptable aqueous composition comprising paclitaxel or a derivative thereof, serum albumin and a pharmaceutically acceptable vehicle, wherein the composition comprises no more than 10% organic solvent and has a pH of about 3.0 to about 4.8.

2. The composition of claim 1, wherein the serum albumin is undefatted.

3. The composition of claim 1, wherein the composition has been lyophilized or lyophilized and then reconstituted from the lyophilized formulation.

4. An optically clear, pharmaceutically acceptable aqueous composition comprising paclitaxel or a derivative thereof, defatted serum albumin and a pharmaceutically acceptable vehicle, wherein the composition comprises about 10% or less organic solvent.

5. The composition as claimed in any one of 1 to 4, wherein at least 70% of the paclitaxel or derivative thereof introduced into the composition is bound to the serum albumin.

6. The composition as claimed in any one of claims 1 to 4, wherein at least 80% of the paclitaxel or derivative thereof into the composition is bound to the serum albumin.

7. The composition as claimed in any one of claims 1 to 4, wherein at least 85% of the paclitaxel or derivative thereof into the composition is bound to the serum albumin.

8. The composition as claimed in any one of claims 1 to 4, wherein at least 90% of the paclitaxel or derivative thereof into the composition is bound to the serum albumin.

9. The composition as claimed in any one of claims 1 to 8, wherein the ratio of paclitaxel or derivative thereof to albumin is at least about 1:5.

10. The composition as claimed in claim 9, wherein the ratio of paclitaxel or derivative thereof to albumin is greater than 1:4.

11. The composition of claim 1, wherein the ratio of paclitaxel or derivative thereof to albumin is at least about 1:4.

12. The composition of claim 1, wherein the ratio of paclitaxel or derivative thereof to albumin is at least about 1:2.

13. The composition of claim 1, wherein the ratio of paclitaxel or derivative thereof to albumin is at least about 1:1.

14. The composition of claim 1, wherein the ratio of paclitaxel or derivative thereof to albumin is at least about 1:1 to about 2:1.

15. The composition as claimed in any one of claims 9 to 14, wherein the concentration of paclitaxel is greater than about 25 μg/ml.

16. The composition as claimed in any one of claims 9 to 14, wherein the concentration of paclitaxel is greater than about 50 μg/ml

17. The composition as claimed in any one of claims 9 to 14, wherein the concentration of paclitaxel is greater than about 100 μg/ml.

18. The composition as claimed in any one of claims 9 to 14, wherein the concentration of paclitaxel is greater than about 200 μg/ml.

19. The composition as claimed in any one of claims 9 to 14, wherein the concentration of paclitaxel is greater than about 300 μg/ml.

20. The composition as claimed in any one of claims 9 to 14, wherein the concentration of paclitaxel is greater than about 400 μg/ml.

21. The composition as claimed in any one of claims 9 to 14, wherein the concentration of paclitaxel is greater than about 500 μg/ml.

22. The composition as claimed in any of claims 1 to 21, wherein the concentration of organic solvent is about 1 to about 10% v/v.

23. The composition of claim 22, wherein the concentration of organic solvent is about 2 to about 8% v/v.

24. The composition of claim 23, wherein the concentration of organic solvent is about 4 to about 6% v/v.

25. The composition of claim 3, wherein the composition is essentially free of organic solvent.

26. The composition as claimed in any of claims 1 to 24, wherein the organic solvent is alcohol.

27. The composition of claim 26, wherein the alcohol is ethanol.

28. The composition as claimed in any of claims 1 to 27, wherein the pH is about 3.0 to about 4.8.

29. The composition of claim 28, wherein the pH is about 4.0 or less.

30. The composition of claim 29, wherein the pH is less than about 4.0.

31. The composition of claim 30, wherein the pH is about 3.4 to about 3.8.

32. The composition of claim 1, wherein the serum albumin is at least about 80% to about 90% monomeric.

33. A lyophilized preparation of an optically clear, pharmaceutically acceptable aqueous composition comprising paclitaxel or a derivative thereof, serum albumin and a pharmaceutically acceptable vehicle, wherein the ratio of paclitaxel or derivative thereof to albumin is about 1:4, and wherein the composition comprises less than 10% organic solvent and has a pH of about 3.0 to about 4.8 upon reconstitution, and wherein at least about 70% of the paclitaxel introduced into the composition is bound to the serum albumin and wherein the paclitaxel concentration in the composition is at least 50 μg/ml.

34. A method of treatment, comprising administering to a patient in a pharmaceutically acceptable form a therapeutically effective amount of a composition as claimed in any of claims 1 to 33.

35. A method of making a composition as claimed in any of claims 1 to 33, comprising the steps of: preparing a solution of the paclitaxel or a derivative thereof; preparing a solution of serum albumin; and slowly combining the solutions, and optionally lyophilizing or optionally lyophilizing and reconstituting the combined solutions.

36. The method of claim 35, wherein the ratio of paclitaxel or derivative thereof to albumin is about 1:1, and the solutions are combined at a temperature below room temperature.

37. The method of claim 35, wherein the ratio or paclitaxel or derivative thereof to albumin is about 1:1, and the solutions are combined at a temperature of about 2 to about 8° C.

38. The method of claim 35, wherein the ratio of paclitaxel or derivative thereof to albumin is about 1:1, and solutions are combined at a temperature of about 4° C.

39. A composition as claimed in any of claims 1 to 33, wherein the desired dose can be administered in a period of less than 3 hours.

40. A composition as claimed in any of claims 1 to 33, wherein the desired dose can be administered in a period of less than 2 hours.

41. The method as claimed in any of claims 35 to 38, wherein the solution of paclitaxel is added dropwise at a controlled rate.

42. The method as claimed in any of claims 35 to 38, wherein the solution of paclitaxel is added at a rate of about 1 ml/minute or slower and the drop size is 8 to 20 μl.

43. A method of treatment, comprising administering to a patient a therapeutically effective amount of an optically clear, pharmaceutically acceptable aqueous composition comprising a hydrophobic drug, a globulin and a pharmaceutically acceptable vehicle, where the drug and the globulin are present in at least about approximately a 1:2 molar ratio.

44. A composition comprising a therapeutically effective amount of an optically clear, pharmaceutically acceptable, aqueous composition comprising a hydrophobic drug, a globulin, and a physiologically acceptable vehicle wherein the drug and globulin are present at about a 1:2 molar ratio and the pH is at or below the pI of the globulin.

45. A method of making an optically clear, pharmaceutically acceptable, aqueous composition of a hydrophobic drug, a globulin, and a physiologically acceptable vehicle, comprising the steps of: preparing a solution of the globulin; preparing a solution of drug; and slowly adding the drug solution to the globulin solution, where the globulin solution is at or below the pI of the globulin.
